(19) United States
(12) Patent Application Publication
(10) Pub. No.: US 2007/0259907 A1
(43) Pub. Date: Nov. 8, 2007

(54) ARYL AND ARYLALKYLENYL SUBSTITUTED THIAZOLOQUINOLINES AND THIAZOLONAPHTHYRIDINES

(76) Inventor: Ryan B. Prince, P.O. BOX 33427 St. Paul Minnesota, MA (US)

Correspondence Address: 3M INNOVATIVE PROPERTIES COMPANY, PO BOX 33427, ST. PAUL, MN, 55133-3427, US

(21) Appl. No.: 11/570,709

(22) PCT Filed: Jun. 17, 2005
(86) PCT No.: PCT/US05/21334

§ 371 Date: Dec. 15, 2006

Related U.S. Application Data
(60) Provisional application No. 60/581,205, filed on Jun. 18, 2004.

(51) Int. Cl. A61K 31/4745; C07D 498/14

(52) U.S. Cl. 514/292; 546/83

(57) ABSTRACT

Thiazoloquinoline and thiazolonaphthyridine compounds having an aryl or arylalkylenyl substituent at the 6-, 7-, 8-, or 9-position, pharmaceutical compositions containing the compounds, intermediates, and methods of making and methods of use of these compounds as immunomodulators, for modulating cytokine biosynthesis in animals and in the treatment of diseases including viral and neoplastic diseases are disclosed.

### RELATED APPLICATIONS

The present invention claims priority to U.S. Provisional Application Ser. No. 60/581205, filed Jun. 18, 2004, which is incorporated herein by reference.

### BACKGROUND OF THE INVENTION

In the 1950's the 1H-imidazo[4,5-c]quinoline ring system was developed, and 1-(6-methoxy-8-quinolinyl)-2-methyl-1H-imidazo[4,5-c]quinoline was synthesized for possible use as an antimalarial agent. Subsequently, syntheses of various substituted 1H-imidazo[4,5-c]quinolines were reported. For example, 1-[2-(4-piperidyl)ethyl]-1H-imidazo[4,5-c]quinoline was synthesized as a possible anticonvulsant and cardiovascular agent. Also, several 2-oxoimidazo[4,5-c]quinolines have been reported.

Certain 1H-imidazo[4,5-c]quinolin-4-amines and 1- and 2-substituted derivatives thereof were later found to be useful as antiviral agents, bronchodilators and immunomodulators. Subsequently, certain substituted 1H-imidazo[4,5-c]pyridin-4-amine, quinolin-4-amine, tetrahydroquinolin-4-amine, naphthyridin-4-amine, and tetrahydronaphthyridin-4-amine compounds as well as certain analogous thiazolo and oxazolo compounds were synthesized and found to be useful as immune response modifiers, rendering them useful in the treatment of a variety of disorders.

There continues to be interest in and a need for compounds that have the ability to modulate the immune response, by induction of cytokine biosynthesis or other mechanisms.

### SUMMARY OF THE INVENTION

The present invention provides a new class of compounds that are useful in modulating cytokine biosynthesis in animals. In one aspect, the present invention provides compounds of the Formula (I): 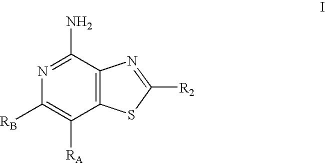
wherein:

R_{A} and R_{B} taken together form a fused benzene ring or fused pyridine ring wherein the benzene ring or pyridine ring is substituted by one R₃ group, or substituted by one R₃ group and one R group; and

R₃ is selected from the group consisting of:
-Z-Ar,
-Z-Ar′—Y—R₄,
-Z-Ar′—X—Y—R₄,
-Z-Ar′—R₅, and
-Z-Ar′—X—R₅;
   wherein R, R₂, Z, Ar, Ar′, X, Y, R₄, and R₅ are as defined below;
   and pharmaceutically acceptable salts thereof.

The compounds of Formula I are useful, for example, as immune response modifiers (IRMs) due to their ability to modulate cytokine biosynthesis (e.g., induce or inhibit the biosynthesis or production of one or more cytokines) and otherwise modulate the immune response when administered to animals. Compounds can be tested, for example, using the test procedures described in the Examples Section. Compounds can be tested for induction of cytokine biosynthesis by incubating human PBMC in a culture with the compound(s) at a concentration range of 30 to 0.014 μM and analyzing for interferon (α) or tumor necrosis factor (α) in the culture supernatant. Compounds can be tested for inhibition of cytokine biosynthesis by incubating mouse macrophage cell line Raw 264.7 in a culture with the compound(s) at a single concentration of, for example, 5 μM and analyzing for tumor necrosis factor (α) in the culture supernatant. The ability to modulate cytokine biosynthesis, for example, induce the biosynthesis of one or more cytokines, makes the compounds useful in the treatment of a variety of conditions such as viral diseases and neoplastic diseases, that are responsive to such changes in the immune response.

In another aspect, the present invention provides pharmaceutical compositions containing an effective amount of a compound of Formula I, and methods of inducing cytokine biosynthesis in animal cells, treating a viral disease in an animal, and/or treating a neoplastic disease in an animal by administering to the animal an effective amount of a compound of Formula I and/or a pharmaceutically acceptable salt thereof.

In another aspect, the invention provides methods of synthesizing the compounds of Formula I and intermediates useful in the synthesis of these compounds.

As used herein, “a,” “an,” “the,” “at least one,” and “one or more” are used interchangeably.

The terms “comprising” and variations thereof do not have a limiting meaning where these terms appear in the description and claims.

The above summary of the present invention is not intended to describe each disclosed embodiment or every implementation of the present invention. The description that follows more particularly exemplifies illustrative embodiments. Guidance is also provided herein through lists of examples, which can be used in various combinations. In each instance, the recited list serves only as a representative group and should not be interpreted as an exclusive or exhaustive list.

### DETAILED DESCRIPTION OF ILLUSTRATIVE EMBODIMENTS OF THE INVENTION

The present invention provides compounds of the following Formulas (I) through (VII): 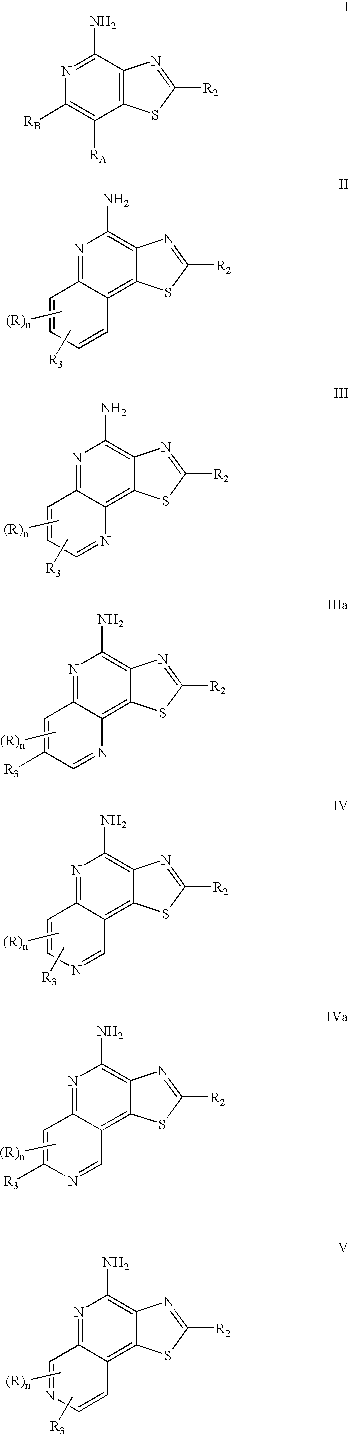 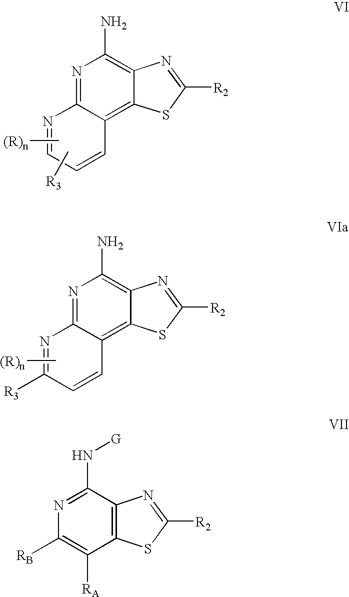
wherein R_{A}, R_{B}, R, R₂, R₃, Z, Ar, Ar′, X, Y, R₄, R₅, G, and n are as defined below.

For any of the compounds presented herein, each one of the following variables (e.g., R_{A}, R_{B}, R, R₂, R₃, Z, Ar, Ar′, X, Y, R₄, R₅, G, n, and so on) in any of its embodiments can be combined with any one or more of the other variables in any of their embodiments and associated with any one of the formulas described herein, as would be understood by one of skill in the art. Each of the resulting combinations of variables is an embodiment of the present invention.

In some embodiments, R_{A} and R_{B} taken together form a fused benzene ring or fused pyridine ring wherein the benzene ring or pyridine ring is substituted by one R₃ group, or substituted by one R₃ group and one R group.

In some embodiments, R is selected from the group consisting of fluoro, chloro, alkyl, alkoxy, hydroxy, and trifluoromethyl.

In some embodiments, R₂ is selected from the group consisting of: hydrogen, alkyl, hydroxyalkylenyl, haloalkylenyl, alkenyl, alkyl-O-alkylenyl, alkyl-O-alkenylenyl, alkenyl-O-alkylenyl, alkenyl-O-alkenylenyl, N(R₈)₂-alkylenyl, N₃-alkylenyl, N(R₈)₂—C(O)—O-alkylenyl, heterocyclyl, heterocyclyl-O-alkylenyl, heterocyclyl-O-alkenylenyl, aryl, aryl-O-alkylenyl, aryl-O-alkenylenyl, heteroaryl, heteroaryl-O-alkylenyl, and heteroaryl-O-alkenylenyl. In some embodiments, R₂ is hydrogen, C₁₋₈ alkyl, or C₁₋₈ alkyl-O—C₁₋₈ alkylenyl. In some embodiments, R₂ is hydrogen, C₁₋₄ alkyl or C₁₋₄ alkyl-O—C₁₋₄ alkylenyl. In some embodiments, R₂ is ethyl, n-propyl, n-butyl, or methoxyethyl.

In some embodiments, R₃ is selected from the group consisting of: -Z-Ar, -Z-Ar′—Y—R₄, -Z-Ar′—X—Y—R₄, -Z-Ar′—R₅, and -Z-Ar′—X_{R}₅. In some embodiments, R₃ is -Z-Ar′—Y—R₄, -Z-Ar′—X—Y—R₄, or -Z-Ar′—R₅. In some embodiments, R₃ is -Z-Ar. In some embodiments, R₃ is -Z-Ar′—Y—R₄. In some embodiments, R₃ is -Z-Ar′—X—Y—R₄. In some embodiments, R₃ is -Z-Ar′—R₅.

In some embodiments R₃ is attached at the 7-position (e.g., as in Figures IIIa, IVa, and Via). In some embodiments, R₃ is selected from the group consisting of phenyl, pyridyl, pyrrolyl, thienyl, and furyl; each of which is substituted by one or more substituents selected from the group consisting of alkenyl, hydroxyalkylenyl, aminoalkylenyl, methylenedioxy, carboxy, and arylalkyleneoxy.

In some embodiments, when R₃ is -Z-Ar′—Y—R₄ and Y is —S—, then R₄ is other than alkyl. In some embodiments, when R₃ is -Z-Ar′—Y—R₄ and Y is —N(R₈)-Q- and R₈ is hydrogen or alkyl and Q is a bond, then R₄ is other than hydrogen or alkyl. In some embodiments, when R₃ is -Z-Ar′—Y—R₄ and Y is —O—, then R₄ is other than hydrogen, alkyl, or haloalkyl. In some embodiments, when R₃ is -Z-Ar′—X—Y—R₄ and X is —CH₂— and Y is —O—, then R₄ is other than alkyl.

In some embodiments, R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo.

In some embodiments, R₄ is selected from the group consisting of hydrogen, alkyl, aryl, arylalkylenyl, heteroaryl, and heteroarylalkylenyl. In some embodiments, R₄ is hydrogen or C₁₋₆ alkyl.

In some embodiments, such as when Y is —S—, R₄ is other than alkyl. In some embodiments, such as when Y is —N(R₈)-Q- and Q is a bond, R₄ is other than hydrogen or alkyl.

In some embodiments, R₅ is

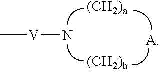

In some embodiments, R₅ is 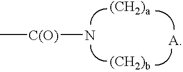
Preferably, in some of these embodiments, A is selected from the group consisting of —O—, —C(O)—, —S(O)₀₋₂—, —CH₂—, and —N(R₈)—. Preferably, in some of these embodiments, A is selected from the group consisting of —O—, —CH₂—, and —N(R₄)—, wherein R₄ is is selected from the group consisting of hydrogen, alkyl, aryl, arylalkylenyl, heteroaryl, and heteroarylalkylenyl.

In some embodiments, R₆ is selected from the group consisting of ═O and ═S.

In some embodiments, R₇ is C₂₋₇ alkylene.

In some embodiments, R₈ is selected from the group consisting of hydrogen, alkyl, alkoxyalkylenyl, and arylalkylenyl. In some embodiments, R₈ is selected from the group consisting of hydrogen, C₁₋₄ alkyl, and alkoxyalkylenyl. In some embodiments, R₈ is hydrogen or alkyl. In some embodiments, R₈ is hydrogen or C₁₋₄ alkyl.

In some embodiments, R₉ is selected from the group consisting of hydrogen and alkyl.

In some embodiments, R₁₀ is independently C₃₋₈ alkylene.

In some embodiments, Ar is selected from the group consisting of aryl and heteroaryl both of which are substituted by one or more substituents independently selected from the group consisting of alkenyl, methylenedioxy, mercapto, carboxy, aryloxy, arylalkoxy, heteroaryloxy, heteroarylalkoxy, and alkyl wherein the alkyl group is substituted by one or more substituents selected from the group consisting of hydroxy, amino, alkylamino, dialkylamino, and carboxy.

In some embodiments, Ar is selected from the group consisting of phenyl, pyridyl, pyrrolyl, thienyl, and furyl; each of which is substituted by one or more substituents selected from the group consisting of alkenyl, methylenedioxy, carboxy, arylalkyleneoxy, and alkyl wherein the alkyl group is substituted by one or more substituents selected from the group consisting of hydroxy, amino, alkylamino, dialkylamino, and carboxy. In some embodiments, Ar is phenyl or pyridyl. In some embodiments the phenyl or pyridyl group is substituted by one HO—C₁₋₄ alkylenyl.

In some embodiments, Ar′ is selected from the group consisting of arylene and heteroarylene both of which can be unsubstituted or can be substituted by one or more substituents independently selected from the group consisting of alkyl, alkenyl, alkoxy, haloalkyl, haloalkoxy, halogen, nitro, hydroxy, hydroxyalkyl, mercapto, cyano, carboxy, formyl, aryl, aryloxy, arylalkoxy, heteroaryl, heteroaryloxy, heteroarylalkoxy, heterocyclyl, heterocyclylalkyl, amino, alkylamino, and dialkylamino. In some embodiments, Ar′ is phenyl or pyridyl. In some embodiments, Ar′ is phenylene.

In some embodiments, A is selected from the group consisting of —O—, —C(O)—, —S(O)₀₋₂—, —CH₂—, and —N(R₄)—. In some embodiments, A is selected from the group consisting of —O—, —C(O)—, —S(O)₀₋₂—, —CH₂—, and —N(R₈)—. In some embodiments, A is selected from the group consisting of —O—, —CH₂—, and —N(R₄)—. In some embodiments, A is —O— or —CH₂—.

In some embodiments, Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—. In some embodiments, Q is a bond. In some embodiments, Q is selected from the group consisting of a bond, —C(O)—, —C(R₆)—N(R₈)—, and —S(O)₂—. In some embodiments, Q is —C(O)— or —S(O)₂—. In some embodiments, Q is selected from the group consisting of —C(O)—, —C(O)—NH—, and —S(O)₂—.

In some embodiments, V is selected from the group consisting of —C(R₆)—, —O—C(R₆)—, —N(R₈)—C(R₆)—, and —S(O)₂—.

In some embodiments, W is selected from the group consisting of a bond, —C(O)—, and —S(O)₂—.

In some embodiments, X is selected from the group consisting of alkylene, alkenylene, alkynylene, arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O— groups. In some embodiments, X is C₁₋₄ alkylene. In some embodiments, X is C₁₋₃ alkylenyl.

In some embodiments, Y is selected from the group consisting of —O—, —S(O)₀₋₂—, —S(O)₂—N(R₈)—, —O—C(R₆)—, —O—C(O)—O—, —N(R₈)-Q-, —C(R₆)—N(R₈)—, —O—C(R₆)—N(R₈)—, —C(R₆)—N(OR₉)—,

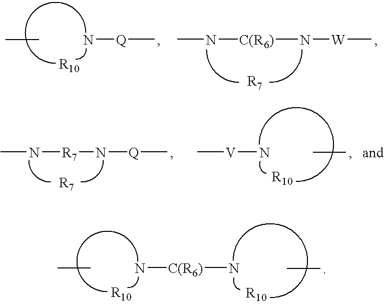

In some embodiments, Y is selected from the group consisting of —S(O)₀₋₂—, —S(O)₂—N(R₈)—, —O—C(R₆)—, —O—C(O)—O—, —N(R₈)-Q-, —C(R₆)—N(R₈)—, —O—C(R₆)—N(R₈)—, —C(R₆)—N(OR₉)—

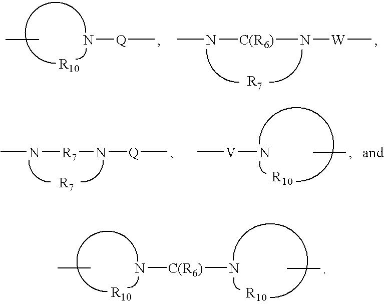

In some embodiments, Y is selected from the group consisting of —S(O)₀₋₂—, —N(R₈)-Q-, —C(R₆)—N(R₈)—, and —C(R₆)—N(OR₉)—. Preferably, in such embodiments, Q is —C(O)— or —S(O)₂—. Preferably, in such embodiments, R₈ is selected from the group consisting of hydrogen, C₁₋₄ alkyl, and alkoxyalkylenyl.

In some embodiments, Y is —S(O)₀₋₂— or —C(O)—N(R₈)—.

In some embodiments, Y is selected from the group consisting of —S(O)₂—, —NH-Q-, and —C(O)—N(R₈)—. Preferably, in such embodiments, Q is selected from the group consisting of —C(O)—, —C(O)—NH—, and —S(O)₂—. Preferably, in such embodiments, R₈ is hydrogen or C₁₋₄ alkyl.

In some embodiments, Y is selected from the group consisting of —S(O)₂—, —N(R₈)-Q-, and —C(O)—N(R₈)—. Preferably, in such embodiments, Q is selected from the group consisting of a bond, —C(O)—, —C(R₆)—N(R₈)—, and —S(O)₂—. Preferably, in such embodiments, R₈ is selected from the group consisting of hydrogen and C₁₋₄ alkyl.

In some embodiments, such as when R₄ is other than alkyl, Y is —S—. In some embodiments, such as when R₄ is other than hydrogen or alkyl, Y is —N(R₈)-Q-. In such embodiments, preferably Q is a bond.

In some embodiments, Z is selected from the group consisting of a bond, alkylene, alkenylene, and alkynylene. In some embodiments, Z is a bond.

In some embodiments, a and b are each an integer from 1 to 6 with the proviso that a+b is ≦7. In some embodiments, a+b is 3 or 4.

In some embodiments, n is 0 or 1. In some embodiments, n is 0.

In one aspect, the present invention provides thiazoloquinoline and thiazolonaphthyridine compounds of the following Formula (I): 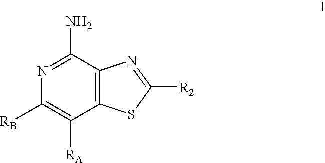
wherein:

R_{A} and R_{B} taken together form a fused benzene ring or fused pyridine ring wherein the benzene ring or pyridine ring is substituted by one R₃ group, or substituted by one R₃ group and one R group;

R₂ is selected from the group consisting of:
hydrogen,
alkyl,
hydroxyalkylenyl,
haloalkylenyl,
alkenyl,
alkyl-O-alkylenyl,
alkyl-O-alkenylenyl,
alkenyl-O-alkylenyl,
alkenyl-O-alkenylenyl,
N(R₈)₂-alkylenyl,
N₃-alkylenyl,
N(R₈)₂—C(O)—O-alkylenyl,
heterocyclyl,
heterocyclyl-O-alkylenyl,
heterocyclyl-O-alkenylenyl,
aryl,
aryl-O-alkylenyl,
aryl-O-alkenylenyl,
heteroaryl,
heteroaryl-O-alkylenyl, and
heteroaryl-O-alkenylenyl;

R₃ is selected from the group consisting of:
-Z-Ar,
-Z-Ar′—Y—R₄,
-Z-Ar′—X—Y—R₄,
-Z-Ar′—R₅, and
-Z-Ar′—X—R₅;

Ar is selected from the group consisting of aryl and heteroaryl both of which are substituted by one or more substituents independently selected from the group consisting of alkenyl, methylenedioxy, mercapto, carboxy, aryloxy, arylalkoxy, heteroaryloxy, heteroarylalkoxy, and alkyl wherein the alkyl group is substituted by one or more substituents selected from the group consisting of hydroxy, amino, alkylamino, dialkylamino, and carboxy;

Ar′ is selected from the group consisting of arylene and heteroarylene both of which can be unsubstituted or can be substituted by one or more substituents independently selected from the group consisting of alkyl, alkenyl, alkoxy, haloalkyl, haloalkoxy, halogen, nitro, hydroxy, hydroxyalkyl, mercapto, cyano, carboxy, formyl, aryl, aryloxy, arylalkoxy, heteroaryl, heteroaryloxy, heteroarylalkoxy, heterocyclyl, heterocyclylalkyl, amino, alkylamino, and dialkylamino;

X is selected from the group consisting of alkylene, alkenylene, alkynylene, arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O— groups;

Y is selected from the group consisting of:
—O—,
—S(O)₀₋₂—,
—S(O)₂—N(R₈)—,
—O—C(R₆)—,
—O—C(O)—O—,
—N(R₈)-Q-,
—C(R₆)—N(R₈)—,
—O—C(R₆)—N(R₈)—,
—C(R₆)—N(OR₉)—,

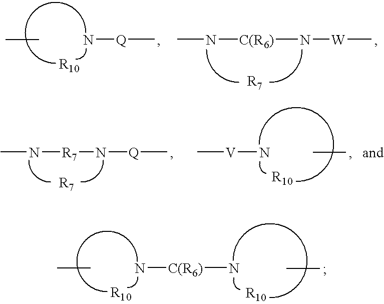

Z is selected from the group consisting of a bond, alkylene, alkenylene, and alkynylene;

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅ is

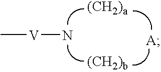

A is selected from the group consisting of —O—, —C(O)—, —S(O)₀₋₂—, —CH₂—, and —N(R₄)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;

V is selected from the group consisting of —C(R₆)—, —O—C(R₆)—, —N(R₈)—C(R₆)—, and —S(O)₂—;

W is selected from the group consisting of a bond, —C(O)—, and —S(O)₂—;

a and b are each an integer from 1 to 6 with the proviso that a+b is ≦7;

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is independently C₃₋₈ alkylene; and

R is selected from the group consisting of fluoro, chloro, alkyl, alkoxy, hydroxy, and trifluoromethyl;

with the proviso that when R₃ is -Z-Ar′—Y—R₄ and Y is —S—, then R₄ is other than alkyl; with the further proviso that when R₃ is -Z-Ar′—Y—R₄ and Y is —N(R₈)-Q- and R₈ is hydrogen or alkyl and Q is a bond, then R₄ is other than hydrogen or alkyl; with the further proviso that when R₃ is -Z-Ar′—Y—R₄ and Y is —O—, then R₄ is other than hydrogen, alkyl, or haloalkyl; and with the further proviso that when R₃ is -Z-Ar′—X—Y—R₄ and X is —CH₂— and Y is —O—, then R₄ is other than alkyl;

or a pharmaceutically acceptable salt thereof.

In one aspect, the present invention provides thiazoloquinoline and thiazolonaphthyridine compounds of the following Formula (I): 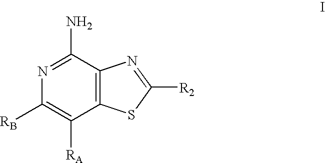
wherein:

R_{A} and R_{B} taken together form a fused benzene ring or fused pyridine ring wherein the benzene ring or pyridine ring is substituted by one R₃ group, or substituted by one R₃ group and one R group;

R₂ is selected from the group consisting of:
hydrogen,
alkyl,
hydroxyalkylenyl,
haloalkylenyl,
alkenyl,
alkyl-O-alkylenyl,
alkyl-O-alkenylenyl,
alkenyl-O-alkylenyl,
alkenyl-O-alkenylenyl,
N(R₈)₂-alkylenyl,
N₃-alkylenyl,
N(R₈)₂—C(O)—O-alkylenyl,
heterocyclyl,
heterocyclyl-O-alkylenyl,
heterocyclyl-O-alkenylenyl,
aryl,
aryl-O-alkylenyl,
aryl-O-alkenylenyl,
heteroaryl,
heteroaryl-O-alkylenyl, and
heteroaryl-O-alkenylenyl;

R₃ is selected from the group consisting of:
-Z-Ar,
-Z-Ar′—Y—R₄,
-Z-Ar′—X—Y—R₄,
-Z-Ar′—R₅, and
-Z-Ar′—X—R₅;

Ar is selected from the group consisting of aryl and heteroaryl both of which are substituted by one or more substituents independently selected from the group consisting of alkenyl, methylenedioxy, mercapto, carboxy, aryloxy, arylalkoxy, heteroaryloxy, heteroarylalkoxy, and alkyl wherein the alkyl group is substituted by one or more substituents selected from the group consisting of hydroxy, amino, alkylamino, dialkylamino, and carboxy;

Ar′ is selected from the group consisting of arylene and heteroarylene both of which can be unsubstituted or can be substituted by one or more substituents independently selected from the group consisting of alkyl, alkenyl, alkoxy, haloalkyl, haloalkoxy, halogen, nitro, hydroxy, hydroxyalkyl, mercapto, cyano, carboxy, formyl, aryl, aryloxy, arylalkoxy, heteroaryl, heteroaryloxy, heteroarylalkoxy, heterocyclyl, heterocyclylalkyl, amino, alkylamino, and dialkylamino;

X is selected from the group consisting of alkylene, alkenylene, alkynylene, arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O— groups;

Y is selected from the group consisting of:
—S(O)₀₋₂—,
—S(O)₂—N(R₈)—,
—O—C(R₆)—,
—O—C(O)—O—,
—N(R₈)-Q-,
—C(R₆)—N(R₈)—,
—O—C(R₆)—N(R₈)—,
—C(R₆)—N(OR₉)—,

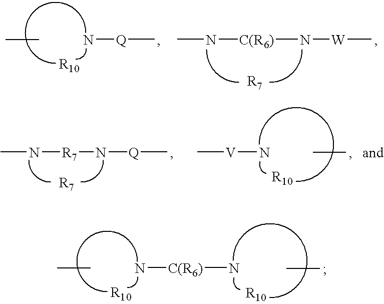

Z is selected from the group consisting of a bond, alkylene, alkenylene, and alkynylene;

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅ is

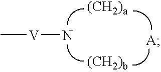

A is selected from the group consisting of —O—, —C(O)—, —S(O)₀₋₂—, —CH₂—, and —N(R₄)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;

V is selected from the group consisting of —C(R₆)—, —O—C(R₆)—, —N(R₈)—C(R₆)—, and —S(O)₂—;

W is selected from the group consisting of a bond, —C(O)—, and —S(O)₂—;

a and b are each an integer from 1 to 6 with the proviso that a+b is ≦7;

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene; and

R is selected from the group consisting of fluoro, chloro, alkyl, alkoxy, hydroxy, and trifluoromethyl;

with the proviso that when Y is —S—, then R₄ is other than alkyl; and with the further proviso that when Y is —N(R₈)-Q- and Q is a bond, then R₄ is other than hydrogen or alkyl;

or a pharmaceutically acceptable salt thereof.

In some embodiments of Formula I, Z is a bond.

In some embodiments of Formula I, R₃ is -Z-Ar. In certain embodiments Z is a bond.

In some embodiments of Formula I, R₃ is -Z-Ar′—Y—R₄, -Z-Ar′—X—Y—R₄, or -Z-Ar—R₅. In certain embodiments Z is a bond. In certain embodiments Y is selected from the group consisting of —S(O)₂—, —C(O)—N(R₈)—, and —N(R₈)-Q-.

In some embodiments of Formula I, R₃ is -Z-Ar′—Y—R₄. In certain embodiments Y is —S(O)₂—, or —C(O)—N(R₈)—, R₈ is selected from the group consisting of hydrogen, C₁₋₄ alkyl, and alkoxyalkylenyl; and R₄ is selected from the group consisting of hydrogen, alkyl, aryl, arylalkylenyl, heteroaryl, and heteroarylalkylenyl. In certain embodiments Z is a bond.

In some embodiments of Formula I, R₅ is 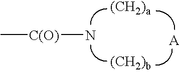
wherein A is selected from the group consisting of —O—, —C(O)—, —S(O)₀₋₂—, —CH₂—, and —N(R₈)—.

For some embodiments of Formula I or any one of the above embodiments, R₂ is selected from the group consisting of hydrogen, C₁₋₈ alkyl, and C₁₋₈ alkyl-O—C₁₋₈ alkylenyl. In certain more specific embodiments R₂ is hydrogen, C₁₋₄ alkyl or C₁₋₄ alkyl-O—C₁₋₄ alkylenyl.

The present invention also provides thiazoloquinoline compounds of the following Formula (II): 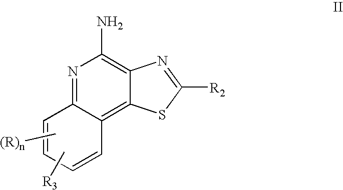
wherein:

R₂ is selected from the group consisting of:
hydrogen,
alkyl,
hydroxyalkylenyl,
haloalkylenyl,
alkenyl,
alkyl-O-alkylenyl,
alkyl-O-alkenylenyl,
alkenyl-O-alkylenyl,
alkenyl-O-alkenylenyl,
N(R₈)₂-alkylenyl,
N₃-alkylenyl,
N(R₈)₂—C(O)—O-alkylenyl,
heterocyclyl,
heterocyclyl-O-alkylenyl,
heterocyclyl-O-alkenylenyl,
aryl,
aryl-O-alkylenyl,
aryl-O-alkenylenyl,
heteroaryl,
heteroaryl-O-alkylenyl, and
heteroaryl-O-alkenylenyl;

R₃ is selected from the group consisting of:
-Z-Ar,
-Z-Ar′—Y—R₄,
-Z-Ar′—X—Y—R₄,
-Z-Ar′—R₅, and
-Z-Ar′—X—R₅;

Ar is selected from the group consisting of aryl and heteroaryl both of which are substituted by one or more substituents independently selected from the group consisting of alkenyl, methylenedioxy, mercapto, carboxy, aryloxy, arylalkoxy, heteroaryloxy, heteroarylalkoxy, and alkyl wherein the alkyl group is substituted by one or more substituents selected from the group consisting of hydroxy, amino, alkylamino, dialkylamino, and carboxy;

Ar′ is selected from the group consisting of arylene and heteroarylene both of which can be unsubstituted or can be substituted by one or more substituents independently selected from the group consisting of alkyl, alkenyl, alkoxy, haloalkyl, haloalkoxy, halogen, nitro, hydroxy, hydroxyalkyl, mercapto, cyano, carboxy, formyl, aryl, aryloxy, arylalkoxy, heteroaryl, heteroaryloxy, heteroarylalkoxy, heterocyclyl, heterocyclylalkyl, amino, alkylamino, and dialkylamino;

X is selected from the group consisting of alkylene, alkenylene, alkynylene, arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O— groups;

Y is selected from the group consisting of:
—O—,
—S(O)₀₋₂—,
—S(O)₂—N(R₈)—,
—O—C(R₆)—,
—O—C(O)—O—,
—N(R₈)-Q-,
—C(R₆)—N(R₈)—,
—O—C(R₆)—N(R₈)—,
—C(R₆)—N(OR₉)—,

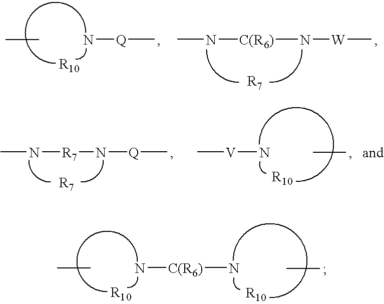

Z is selected from the group consisting of a bond, alkylene, alkenylene, and alkynylene;

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅ is

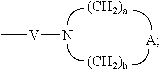

A is selected from the group consisting of —O—, —C(O)—, —S(O)₀₋₂—, —CH₂—, and —N(R₄)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;

V is selected from the group consisting of —C(R₆)—, —O—C(R₆)—, —N(R₈)—C(R₆)—, and —S(O)₂—;

W is selected from the group consisting of a bond, —C(O)—, and —S(O)₂—;

a and b are each an integer from 1 to 6 with the proviso that a+b is ≦7;

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is independently C₃₋₈ alkylene;

R is selected from the group consisting of fluoro, chloro, alkyl, alkoxy, hydroxy, and trifluoromethyl; and

n is 0 or 1;

with the proviso that when R₃ is -Z-Ar′—Y—R₄ and Y is —S—, then R₄ is other than alkyl; with the further proviso that when R₃ is -Z-Ar′—Y—R₄ and Y is —N(R₈)-Q- and R₈ is hydrogen or alkyl and Q is a bond, then R₄ is other than hydrogen or alkyl; with the further proviso that when R₃ is -Z-Ar′—Y—R₄ and Y is —O—, then R₄ is other than hydrogen, alkyl, or haloalkyl; and with the further proviso that when R₃ is -Z-Ar′—X—Y—R₄ and X is —CH₂— and Y is —O—, then R₄ is other than alkyl;

or a pharmaceutically acceptable salt thereof.

The present invention also provides thiazoloquinoline compounds of the following Formula (II): 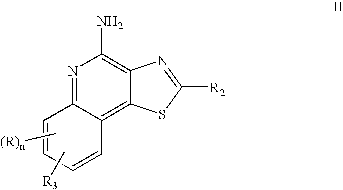
wherein:

R₂ is selected from the group consisting of:
hydrogen,
alkyl,
hydroxyalkylenyl,
haloalkylenyl,
alkenyl,
alkyl-O-alkylenyl,
alkyl-O-alkenylenyl,
alkenyl-O-alkylenyl,
alkenyl-O-alkenylenyl,
N(R₈)₂-alkylenyl,
N₃-alkylenyl,
N(R₈)₂—C(O)—O-alkylenyl,
heterocyclyl,
heterocyclyl-O-alkylenyl,
heterocyclyl-O-alkenylenyl,
aryl,
aryl-O-alkylenyl,
aryl-O-alkenylenyl,
heteroaryl,
heteroaryl-O-alkylenyl, and
heteroaryl-O-alkenylenyl;

R₃ is selected from the group consisting of:
-Z-Ar,
-Z-Ar′—Y—R₄,
-Z-Ar′—X—Y—R₄,
-Z-Ar′—R₅, and
-Z-Ar′—X—R₅;

Ar is selected from the group consisting of aryl and heteroaryl both of which are substituted by one or more substituents independently selected from the group consisting of alkenyl, methylenedioxy, mercapto, carboxy, aryloxy, arylalkoxy, heteroaryloxy, heteroarylalkoxy, and alkyl wherein the alkyl group is substituted by one or more substituents selected from the group consisting of hydroxy, amino, alkylamino, dialkylamino, and carboxy;

Ar′ is selected from the group consisting of arylene and heteroarylene both of which can be unsubstituted or can be substituted by one or more substituents independently selected from the group consisting of alkyl, alkenyl, alkoxy, haloalkyl, haloalkoxy, halogen, nitro, hydroxy, hydroxyalkyl, mercapto, cyano, carboxy, formyl, aryl, aryloxy, arylalkoxy, heteroaryl, heteroaryloxy, heteroarylalkoxy, heterocyclyl, heterocyclylalkyl, amino, alkylamino, and dialkylamino;

X is selected from the group consisting of alkylene, alkenylene, alkynylene, arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O— groups;

Y is selected from the group consisting of:
—S(O)₀₋₂—,
—S(O)₂—N(R₈)—,
—O—C(R₆)—,
—O—C(O)—O—,
—N(R₈)-Q-,
—C(R₆)—N(R₈)—,
—O—C(R₆)—N(R₈)—,
—C(R₆)—N(OR₉)—,

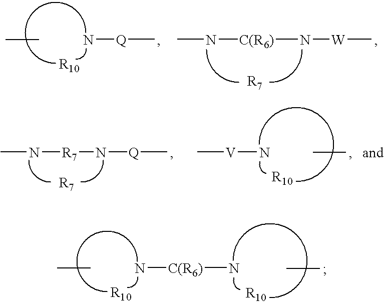

Z is selected from the group consisting of a bond, alkylene, alkenylene, and alkynylene;

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅ is

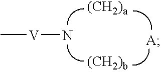

A is selected from the group consisting of —O—, —C(O)—, —S(O)₀₋₂—, —CH₂—, and —N(R₄)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;

V is selected from the group consisting of —C(R₆)—, —O—C(R₆)—, —N(R₈)—C(R₆)—, and —S(O)₂—;

W is selected from the group consisting of a bond, —C(O)—, and —S(O)₂—;

a and b are each an integer from 1 to 6 with the proviso that a+b is ≦7;

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

R is selected from the group consisting of fluoro, chloro, alkyl, alkoxy, hydroxy, and trifluoromethyl; and

n is 0 or 1;

with the proviso that when Y is —S—, then R₄ is other than alkyl; and with the further proviso that when Y is —N(R₈)-Q- and Q is a bond, then R₄ is other than hydrogen or alkyl;

or a pharmaceutically acceptable salt thereof.

In some embodiments of Formula II, Z is a bond.

In some embodiments of Formula II, R₃ is -Z-Ar. In certain embodiments Z is a bond. In certain embodiments R₃ is selected from the group consisting of phenyl, pyridyl, pyrrolyl, thienyl, and furyl; each of which is substituted by one or more substituents selected from the group consisting of alkenyl, hydroxyalkylenyl, aminoalkylenyl, methylenedioxy, carboxy, and arylalkyleneoxy.

In some embodiments of Formula II, R₃ is -Z-Ar′—Y—R₄, -Z-Ar′—X—Y—R₄, or -Z-Ar—R₅. In certain embodiments Z is a bond. In certain embodiments Ar′ is phenyl or pyridyl; Y is selected from the group consisting of —S(O)₀₋₂—, —N(R₈)-Q-, —C(R₆)—N(R₈)—, and —C(R₆)—N(OR₉)—; wherein Q is selected from the group consisting of —C(O)— and —S(O)₂—; and R₈ is selected from the group consisting of hydrogen, C₁₋₄ alkyl, and alkoxyalkylenyl; X is C₁₋₄ alkylene; R₄ is selected from the group consisting of hydrogen, alkyl, aryl, arylalkylenyl, heteroaryl, and heteroarylalkylenyl; and R₅ is

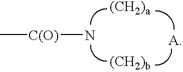

In some embodiments of Formula II, R₃ is -Z-Ar′—Y—R₄. In certain embodiments Y is selected from the group consisting of —S(O)₂—, —C(O)—N(R₈)—, and —N(R₈)-Q-. In certain embodiments Y is —S(O)₂—, or —C(O)—N(R₈)—, R₈ is selected from the group consisting of hydrogen, C₁₋₄ alkyl, and alkoxyalkylenyl; and R₄ is selected from the group consisting of hydrogen, alkyl, aryl, arylalkylenyl, heteroaryl, and heteroarylalkylenyl. In certain embodiments Z is a bond.

For some embodiments of Formula II or any one of the above embodiments of Formula II, R₂ is selected from the group consisting of hydrogen, C₁₋₈ alkyl, and C₁₋₈ alkyl-O—C₁₋₈ alkylenyl. In certain more specific embodiments R₂ is hydrogen, C₁₋₄ alkyl or C₁₋₄ alkyl-O—C₁₋₄ alkylenyl.

For some embodiments of Formula II or any one of the above embodiments of Formula II, R₃ is attached at the 7-position.

For some embodiments of Formula II or any one of the above embodiments of Formula II, n is 0.

The present invention also provides thiazolonaphthyridine compounds of the following Formula (III): 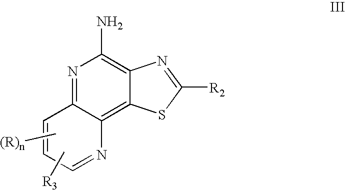
wherein:

R₂ is selected from the group consisting of:
hydrogen,
alkyl,
hydroxyalkylenyl,
haloalkylenyl,
alkenyl,
alkyl-O-alkylenyl,
alkyl-O-alkenylenyl,
alkenyl-O-alkylenyl,
alkenyl-O-alkenylenyl,
N(R₈)₂-alkylenyl,
N₃-alkylenyl,
N(R₈)₂—C(O)—O-alkylenyl,
heterocyclyl,
heterocyclyl-O-alkylenyl,
heterocyclyl-O-alkenylenyl,
aryl,
aryl-O-alkylenyl,
aryl-O-alkenylenyl,
heteroaryl,
heteroaryl-O-alkylenyl, and
heteroaryl-O-alkenylenyl;

R₃ is selected from the group consisting of:
-Z-Ar,
-Z-Ar′—Y—R₄,
-Z-Ar′—X—Y—R₄,
-Z-Ar′—R₅, and
-Z-Ar′—X—R₅;

Ar is selected from the group consisting of aryl and heteroaryl both of which are substituted by one or more substituents independently selected from the group consisting of alkenyl, methylenedioxy, mercapto, carboxy, aryloxy, arylalkoxy, heteroaryloxy, heteroarylalkoxy, and alkyl wherein the alkyl group is substituted by one or more substituents selected from the group consisting of hydroxy, amino, alkylamino, dialkylamino, and carboxy;

Ar′ is selected from the group consisting of arylene and heteroarylene both of which can be unsubstituted or can be substituted by one or more substituents independently selected from the group consisting of alkyl, alkenyl, alkoxy, haloalkyl, haloalkoxy, halogen, nitro, hydroxy, hydroxyalkyl, mercapto, cyano, carboxy, formyl, aryl, aryloxy, arylalkoxy, heteroaryl, heteroaryloxy, heteroarylalkoxy, heterocyclyl, heterocyclylalkyl, amino, alkylamino, and dialkylamino;

X is selected from the group consisting of alkylene, alkenylene, alkynylene, arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O— groups;

Y is selected from the group consisting of:
—O—,
—S(O)₀₋₂—,
—S(O)₂—N(R₈)—,
—O—C(R₆)—,
—O—C(O)—O—,
—N(R₈)-Q-,
—C(R₆)—N(R₈)—,
—O—C(R₆)—N(R₈)—,
—C(R₆)-N(OR₉)—,

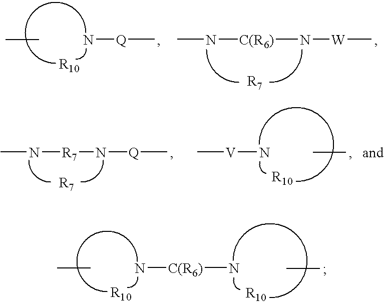

Z is selected from the group consisting of a bond, alkylene, alkenylene, and alkynylene;

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅ is

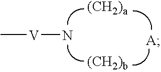

A is selected from the group consisting of —O—, —C(O)—, —S(O)₀₋₂—, —CH₂—, and —N(R₄)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;

V is selected from the group consisting of —C(R₆)—, —O—C(R₆)—, —N(R₈)—C(R₆)—, and —S(O)₂—;

W is selected from the group consisting of a bond, —C(O)—, and —S(O)₂—;

a and b are each an integer from 1 to 6 with the proviso that a+b is ≦7;

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is independently C₃₋₈ alkylene;

R is selected from the group consisting of fluoro, chloro, alkyl, alkoxy, hydroxy, and trifluoromethyl; and

n is 0 or 1;

with the proviso that when R₃ is -Z-Ar′—Y—R₄ and Y is —S—, then R₄ is other than alkyl; with the further proviso that when R₃ is -Z-Ar′—Y—R₄ and Y is —N(R₈)-Q- and R₈ is hydrogen or alkyl and Q is a bond, then R₄ is other than hydrogen or alkyl; with the further proviso that when R₃ is -Z-Ar′—Y—R₄ and Y is —O—, then R₄ is other than hydrogen, alkyl, or haloalkyl; and with the further proviso that when R₃ is -Z-Ar′—X—Y—R₄ and X is —CH₂— and Y is —O—, then R₄ is other than alkyl;

or a pharmaceutically acceptable salt thereof.

The present invention also provides thiazolonaphthyridine compounds of the following Formula (III): 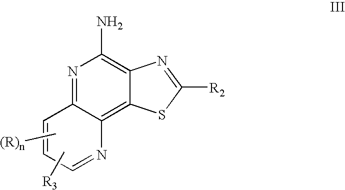
wherein:

R₂ is selected from the group consisting of:
hydrogen,
alkyl,
hydroxyalkylenyl,
haloalkylenyl,
alkenyl,
alkyl-O-alkylenyl,
alkyl-O-alkenylenyl,
alkenyl-O-alkylenyl,
alkenyl-O-alkenylenyl,
N(R₈)₂-alkylenyl,
N₃-alkylenyl,
N(R₈)₂—C(O)—O-alkylenyl,
heterocyclyl,
heterocyclyl-O-alkylenyl,
heterocyclyl-O-alkenylenyl,
aryl,
aryl-O-alkylenyl,
aryl-O-alkenylenyl,
heteroaryl,
heteroaryl-O-alkylenyl, and
heteroaryl-O-alkenylenyl;

R₃ is selected from the group consisting of:
-Z-Ar,
-Z-Ar′—Y—R₄,
-Z-Ar′—X—Y—R₄,
-Z-Ar′—R₅, and
-Z-Ar′—X—R₅;

Ar is selected from the group consisting of aryl and heteroaryl both of which are substituted by one or more substituents independently selected from the group consisting of alkenyl, methylenedioxy, mercapto, carboxy, aryloxy, arylalkoxy, heteroaryloxy, heteroarylalkoxy, and alkyl wherein the alkyl group is substituted by one or more substituents selected from the group consisting of hydroxy, amino, alkylamino, dialkylamino, and carboxy;

Ar′ is selected from the group consisting of arylene and heteroarylene both of which can be unsubstituted or can be substituted by one or more substituents independently selected from the group consisting of alkyl, alkenyl, alkoxy, haloalkyl, haloalkoxy, halogen, nitro, hydroxy, hydroxyalkyl, mercapto, cyano, carboxy, formyl, aryl, aryloxy, arylalkoxy, heteroaryl, heteroaryloxy, heteroarylalkoxy, heterocyclyl, heterocyclylalkyl, amino, alkylamino, and dialkylamino;

X is selected from the group consisting of alkylene, alkenylene, alkynylene, arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O— groups;

Y is selected from the group consisting of:
—S(O)₀₋₂—,
—S(O)₂—N(R₈)—,
—O—C(R₆)—,
—O—C(O)—O—,
—N(R₈)-Q-,
—C(R₆)—N(R₈)—,
—O—C(R₆)—N(R₈)—,
—C(R₆)—N(OR₉)—,

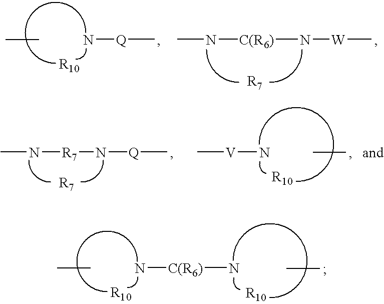

Z is selected from the group consisting of a bond, alkylene, alkenylene, and alkynylene;

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅ is

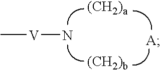

A is selected from the group consisting of —O—, —C(O)—, —S(O)₀₋₂—, —CH₂—, and —N(R₄)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;

V is selected from the group consisting of —C(R₆)—, —O—C(R₆)—, —N(R₈)—C(R₆)—, and —S(O)₂—;

W is selected from the group consisting of a bond, —C(O)—, and —S(O)₂—;

a and b are each an integer from 1 to 6 with the proviso that a+b is ≦7;

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

R is selected from the group consisting of fluoro, chloro, alkyl, alkoxy, hydroxy, and trifluoromethyl; and

n is 0 or 1;

with the proviso that when Y is —S—, then R₄ is other than alkyl; and with the further proviso that when Y is —N(R₈)-Q- and Q is a bond, then R₄ is other than hydrogen or alkyl;

or a pharmaceutically acceptable salt thereof.

The present invention also provides thiazolonaphthyridine compounds of the following Formulas (IV, V, and VI): 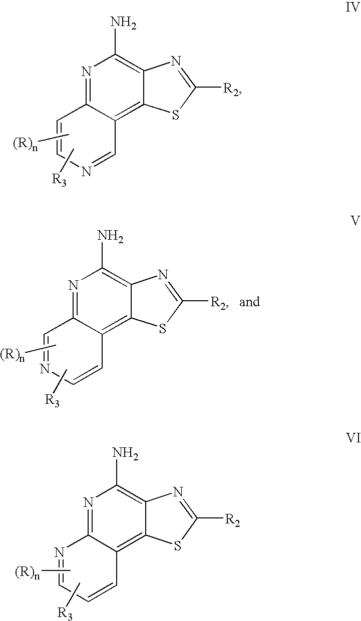
wherein:

R₂ is selected from the group consisting of:
hydrogen,
alkyl,
hydroxyalkylenyl,
haloalkylenyl,
alkenyl,
alkyl-O-alkylenyl,
alkyl-O-alkenylenyl,
alkenyl-O-alkylenyl,
alkenyl-O-alkenylenyl,
N(R₈)₂-alkylenyl,
N₃-alkylenyl,
N(R₈)₂—C(O)—O-alkylenyl,
heterocyclyl,
heterocyclyl-O-alkylenyl,
heterocyclyl-O-alkenylenyl,
aryl,
aryl-O-alkylenyl,
aryl-O-alkenylenyl,
heteroaryl,
heteroaryl-O-alkylenyl, and
heteroaryl-O-alkenylenyl;

R₃ is selected from the group consisting of:
-Z-Ar,
-Z-Ar′—Y—R₄,
-Z-Ar′—X—Y—R₄,
-Z-Ar′—R₅, and
-Z-Ar′—X—R₅;

Ar is selected from the group consisting of aryl and heteroaryl both of which are substituted by one or more substituents independently selected from the group consisting of alkenyl, methylenedioxy, mercapto, carboxy, aryloxy, arylalkoxy, heteroaryloxy, heteroarylalkoxy, and alkyl wherein the alkyl group is substituted by one or more substituents selected from the group consisting of hydroxy, amino, alkylamino, dialkylamino, and carboxy;

Ar′ is selected from the group consisting of arylene and heteroarylene both of which can be unsubstituted or can be substituted by one or more substituents independently selected from the group consisting of alkyl, alkenyl, alkoxy, haloalkyl, haloalkoxy, halogen, nitro, hydroxy, hydroxyalkyl, mercapto, cyano, carboxy, formyl, aryl, aryloxy, arylalkoxy, heteroaryl, heteroaryloxy, heteroarylalkoxy, heterocyclyl, heterocyclylalkyl, amino, alkylamino, and dialkylamino;

X is selected from the group consisting of alkylene, alkenylene, alkynylene, arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O— groups;

Y is selected from the group consisting of:
—S(O)₀₋₂—,
—S(O)₂—N(R₈)—,
—O—C(R₆)—,
—O—C(O)—O—,
—N(R₈)-Q-,
—C(R₆)—N(R₈)—,
—O—C(R₆)—N(R₈)—,
—C(R₆)—N(OR₉)—,

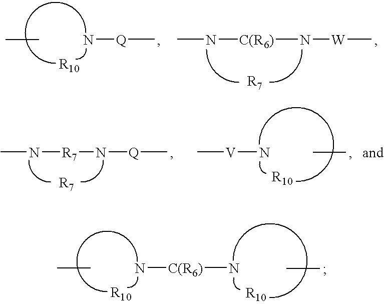

Z is selected from the group consisting of a bond, alkylene, alkenylene, and alkynylene;

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅ is

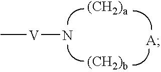

A is selected from the group consisting of —O—, —C(O)—, —S(O)₀₋₂—, —CH₂—, and —N(R₄)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;

V is selected from the group consisting of —C(R₆)—, —O—C(R₆)—, —N(R₈)—C(R₆)—, and —S(O)₂—;

W is selected from the group consisting of a bond, —C(O)—, and —S(O)₂—;

a and b are each an integer from 1 to 6 with the proviso that a+b is ≦7;

R₆ is selected from the group consisting of ═O and ═S;

R₇ is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is C₃₋₈ alkylene;

R is selected from the group consisting of fluoro, chloro, alkyl, alkoxy, hydroxy, and trifluoromethyl; and

n is 0 or 1;

with the proviso that when Y is —S—, then R₄ is other than alkyl; and with the further proviso that when Y is —N(R₈)-Q- and Q is a bond, then R₄ is other than hydrogen or alkyl;

or a pharmaceutically acceptable salt thereof.

In some embodiments of Formulas III, IV, V, and VI, Z is a bond.

In some embodiments of Formulas III, IV, V, and VI, R₃ is -Z-Ar. In certain embodiments Z is a bond. In certain embodiments R₃ is selected from the group consisting of phenyl, pyridyl, pyrrolyl, thienyl, and furyl; each of which is substituted by one or more substituents selected from the group consisting of alkenyl, hydroxyalkylenyl, aminoalkylenyl, methylenedioxy, carboxy, and arylalkyleneoxy.

In some embodiments of Formulas III, IV, V, and VI, R₃ is -Z-Ar′—Y—R₄, -Z-Ar′—X—Y—R₄, or -Z-Ar—R₅. In certain embodiments Z is a bond. In certain embodiments Ar′ is phenyl or pyridyl; Y is selected from the group consisting of —S(O)₀₋₂—, —N(R₈)-Q-, —C(R₆)—N(R₈)—, and —C(R₆)—N(OR₉)—; wherein Q is selected from the group consisting of —C(O)—and —S(O)₂—; and R₈ is selected from the group consisting of hydrogen, C₁₋₄ alkyl, and alkoxyalkylenyl; X is C₁₋₄ alkylene; R₄ is selected from the group consisting of hydrogen, alkyl, aryl, arylalkylenyl, heteroaryl, and heteroarylalkylenyl; and R₅ is

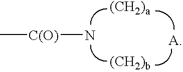

In some embodiments of Formulas III, IV, V, and VI, R₃ is -Z-Ar′—Y—R₄. In certain embodiments Y is selected from the group consisting of —S(O)₂—, —C(O)—N(R₈)—, and —N(R₈)-Q-. In certain embodiments Y is —S(O)₂—, or —C(O)—N(R₈)—, R₈ is selected from the group consisting of hydrogen, C₁₋₄ alkyl, and alkoxyalkylenyl; and R₄ is selected from the group consisting of hydrogen, alkyl, aryl, arylalkylenyl, heteroaryl, and heteroarylalkylenyl. In certain embodiments Z is a bond.

For some embodiments of Formulas III, IV, V, and VI or any one of the above embodiments of Formulas III, IV, V, and VI, R₂ is selected from the group consisting of hydrogen, C₁₋₈ alkyl, and C₁₋₈ alkyl-O—C₁₋₈ alkylenyl. In certain more specific embodiments R₂ is hydrogen, C₁₋₄ alkyl or C₁₋₄ alkyl-O—C₁₋₄ alkylenyl.

In some embodiments of Formulas III, IV, and VI and any one of the above embodiments described above for Formulas III, IV, and VI, R₃ is attached at the 7-position. That is, the thiazolonaphthyridines selected from Formulas III, IV, V, and VI are the compounds of the formulas (IIIa, IVa, and VIa):

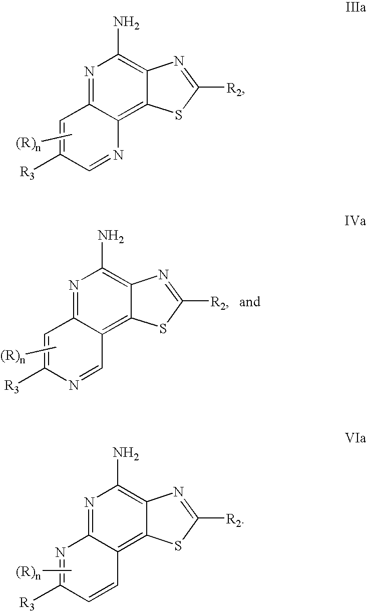

In some embodiments, the thiazolonaphthyridines selected from Formulas III, IV, V, and VI or any one of the above embodiments described above for Formula III, IV, V, and VI are the compounds of the formula (III):

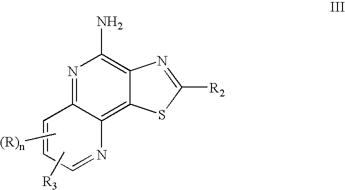

In some embodiments of Formulas III, IV, V, and VI or any one of the above embodiments of Formulas III, IV, V, and VI, n is 0.

In some embodiments of any of the formulas presented herein, R₃ is -Z-Ar′—Y—R₄, -Z-Ar′—X—Y—R₄, or -Z-Ar′—R₅. Preferably, in such embodiments, Ar′ is phenyl or pyridyl. Preferably, in such embodiments, X is C₁₋₄ alkylene. Preferably, in such embodiments, R₄ is selected from the group consisting of hydrogen, alkyl, aryl, arylalkylenyl, heteroaryl, and heteroarylalkylenyl. Preferably, in such embodiments, R₅ is 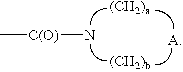
Preferably, in such embodiments, Y is selected from the group consisting of —S(O)₀₋₂—, —N(R₈)-Q-, —C(R₆)—N(R₈)—, and —C(R₆)—N(OR₉)—, wherein, preferably, Q is —C(O)— or —S(O)₂—, and R₈ is selected from the group consisting of hydrogen, C₁₋₄ alkyl, and alkoxyalkylenyl. Alternatively, Y is selected from the group consisting of —S(O)₂—, —C(O)—N(R₈)—, and —N(R₈)-Q-.

In some embodiments of any of the formulas presented herein, R₃ is -Z-Ar. Preferably, in such embodiments, Ar is selected from the group consisting of phenyl, pyridyl, pyrrolyl, thienyl, and furyl; each of which is substituted by one or more substituents selected from the group consisting of alkenyl, methylenedioxy, carboxy, arylalkyleneoxy, and alkyl wherein the alkyl group is substituted by one or more substituents selected from the group consisting of hydroxy, amino, alkylamino, dialkylamino, and carboxy. In such embodiments, more preferably, Ar is phenyl or pyridyl, and even more preferably, the phenyl or pyridyl group is substituted by one HO—C₁₋₄alkylenyl.

In some embodiments of any of the formulas presented herein, R₃ is -Z-Ar′—R₅. Preferably, in such embodiments, Ar′ is phenylene; and R₅ is 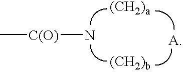
Preferably, in such embodiments, A is selected from the group consisting of —O—, —CH₂—, and —N(R₄)—, and more preferably, A is —O— or —CH₂—. Preferably, in such embodiments, R₄ is is selected from the group consisting of hydrogen, alkyl, aryl, arylalkylenyl, heteroaryl, and heteroarylalkylenyl. Preferably, in such embodiments, particularly when A is —O— or —CH₂—, a+b is 3 or 4.

In some embodiments of any of the formulas presented herein, R₃ is -Z-Ar′—Y—R₄. In such embodiments, preferably, R₄ is selected from the group consisting of hydrogen, alkyl, aryl, arylalkylenyl, heteroaryl, and heteroarylalkylenyl, and more preferably, R₄ is hydrogen or C₁₋₆ alkyl. Preferably, in such embodiments (particularly when R₄ is selected from the group consisting of hydrogen, alkyl, aryl, arylalkylenyl, heteroaryl, and heteroarylalkylenyl), Y is selected from the group consisting of —S(O)₂—, —N(R₈)-Q-, and —C(O)—N(R₈)—, wherein, preferably, Q is selected from the group consisting of a bond, —C(O)—, —C(R₆)—N(R₈)—, and —S(O)₂—, and R₈ is hydrogen or C₁₋₄ alkyl. Alternatively (particularly, when R₄ is hydrogen or C₁₋₆ alkyl), Y is selected from the group consisting of —S(O)₂—, —NH-Q-, and —C(O)—N(R₈)—, wherein, preferably, Q is selected from the group consisting of —C(O)—, —C(O)—NH—, and —S(O)₂—, and R₈ is hydrogen or C₁₋₄ alkyl. In such embodiments, preferably, Ar′ is phenylene. Alternatively (particularly when R₄ is selected from the group consisting of hydrogen, alkyl, aryl, arylalkylenyl, heteroaryl, and heteroarylalkylenyl), Y is —S(O)₀₋₂—, or —C(O)—N(R₈)—, wherein, preferably, R₈ is selected from the group consisting of hydrogen, C₁₋₄ alkyl, and alkoxyalkylenyl.

In some embodiments of any of the formulas presented herein, R₃ is -Z-Ar′—X—Y—R₄. In such embodiments, preferably, X is C₁₋₃ alkylenyl. In such embodiments, preferably, R₄ is selected from the group consisting of hydrogen, alkyl, aryl, arylalkylenyl, heteroaryl, and heteroarylalkylenyl, and more preferably, R₄ is hydrogen or C₁₋₆ alkyl. In such embodiments, preferably (particularly when R₄ is selected from the group consisting of hydrogen, alkyl, aryl, arylalkylenyl, heteroaryl, and heteroarylalkylenyl), Y is selected from the group consisting of —S(O)₂—, —N(R₈)-Q-, and —C(O)—N(R₈)—, wherein, preferably, Q is selected from the group consisting of a bond, —C(O)—, —C(R₆)—N(R₈)—, and —S(O)₂—, and R₈ is hydrogen or C₁₋₄ alkyl. Preferably (particularly when R₄ is hydrogen or C₁₋₆ alkyl), Y is selected from the group consisting of —S(O)₂—, —NH-Q-, and —C(O)—N(R₈)—, wherein, preferably, Q is selected from the group consisting of —C(O)—, —C(O)—NH—, and —S(O)₂—, and R₈ is hydrogen or C₁₋₄ alkyl. In such embodiments, preferably, Ar′ is phenylene.

In one aspect, the present invention provides a compound of the Formula (VII): 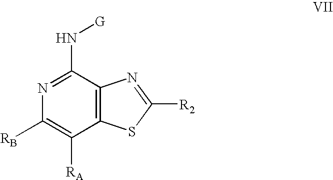
wherein:

R_{A} and R_{B} taken together form a fused benzene ring or fused pyridine ring wherein the benzene ring or pyridine ring is substituted by one R₃ group, or substituted by one R₃ group and one R group;

R₂ is selected from the group consisting of:
hydrogen,
alkyl,
hydroxyalkylenyl,
haloalkylenyl,
alkenyl,
alkyl-O-alkylenyl,
alkyl-O-alkenylenyl,
alkenyl-O-alkylenyl,
alkenyl-O-alkenylenyl,
N(R₈)₂-alkylenyl,
N₃-alkylenyl,
N(R₈)₂—C(O)—O-alkylenyl,
heterocyclyl,
heterocyclyl-O-alkylenyl,
heterocyclyl-O-alkenylenyl,
aryl,
aryl-O-alkylenyl,
aryl-O-alkenylenyl,
heteroaryl,
heteroaryl-O-alkylenyl, and
heteroaryl-O-alkenylenyl;

R₃ is selected from the group consisting of:
-Z-Ar,
-Z-Ar′—Y—R₄,
-Z-Ar′—X—Y—R₄,
-Z-Ar′—R₅, and
-Z-Ar′—X—R₅;

Ar is selected from the group consisting of aryl and heteroaryl both of which are substituted by one or more substituents independently selected from the group consisting of alkenyl, methylenedioxy, mercapto, carboxy, aryloxy, arylalkoxy, heteroaryloxy, heteroarylalkoxy, and alkyl wherein the alkyl group is substituted by one or more substituents selected from the group consisting of hydroxy, amino, alkylamino, dialkylamino, and carboxy;

Ar′ is selected from the group consisting of arylene and heteroarylene both of which can be unsubstituted or can be substituted by one or more substituents independently selected from the group consisting of alkyl, alkenyl, alkoxy, haloalkyl, haloalkoxy, halogen, nitro, hydroxy, hydroxyalkyl, mercapto, cyano, carboxy, formyl, aryl, aryloxy, arylalkoxy, heteroaryl, heteroaryloxy, heteroarylalkoxy, heterocyclyl, heterocyclylalkyl, amino, alkylamino, and dialkylamino;

X is selected from the group consisting of alkylene, alkenylene, alkynylene, arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O— groups;

Y is selected from the group consisting of:
—O—,
—S(O)₀₋₂—,
—S(O)₂—N(R₈)—,
—O—C(R₆)—,
—O—C(O)—O—,
—N(R₈)-Q-,
—C(R₆)—N(R₈)—,
—O—C(R₆)—N(R₈)—,
—C(R₆)—N(OR₉)—,

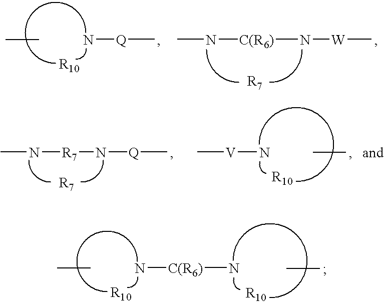

Z is selected from the group consisting of a bond, alkylene, alkenylene, and alkynylene;

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅ is

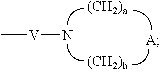

A is selected from the group consisting of —O—, —C(O)—, —S(O)₀₋₂—, —CH₂—, and —N(R₄)—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;

V is selected from the group consisting of —C(R₆)—, —O—C(R₆)—, —N(R₈)—C(R₆)—, and —S(O)₂—;

W is selected from the group consisting of a bond, —C(O)—, and —S(O)₂—;

a and b are each an integer from 1 to 6 with the proviso that a+b is ≦7;

R₆is selected from the group consisting of ═O and ═S;

R₇is C₂₋₇ alkylene;

R₈ is selected from the group consisting of hydrogen, alkyl, alkoxyalkylenyl, and arylalkylenyl;

R₉ is selected from the group consisting of hydrogen and alkyl;

R₁₀ is independently C₃₋₈ alkylene; and

R is selected from the group consisting of fluoro, chloro, alkyl, alkoxy, hydroxy, and trifluoromethyl;

G is selected from the group consisting of:
—C(O)—R′,
α-aminoacyl,
α-aminoacyl-α-aminoacyl,
—C(O)—O—R′,
—C(O)—N(R″)R′,
—C(═NY′)—R′,
—CH(OH)—C(O)—OY′,
—CH(OC₁₋₄ alkyl)Y₀,
—CH₂Y₁, and
—CH(CH₃)Y₁;

R′ and R″ are independently selected from the group consisting of C₁₋₁₀ alkyl, C₃₋₇ cycloalkyl, and benzyl, each of which may be unsubstituted or substituted by one or more substitutents selected from the group consisting of halogen, hydroxy, nitro, cyano, carboxy, C₁₋₆ alkyl, C₁₋₄ alkoxy, aryl, heteroaryl, arylC₁₋₄ alkylenyl, heteroarylC₁₋₄ alkylenyl, haloC₁₋₄ alkylenyl, haloC₁₋₄ alkoxy, —O—C(O)—CH₃, —C(O)—O—CH₃, —C(O)—NH₂, —O—CH₂—C(O)—NH₂, —NH₂, and —S(O)₂—NH₂, with the proviso that R″ can also be hydrogen;

α-aminoacyl is an acyl group derived from an amino acid selected from the group consisting of racemic, D-, and L-amino acids;

Y′ is selected from the group consisting of hydrogen, C₁₋₆ alkyl, and benzyl;

Y₀ is selected from the group consisting of C₁₋₆ alkyl, carboxyC₁₋₆ alkylenyl, aminoC₁₋₄ alkylenyl, mono-N—C₁₋₆ alkylaminoC₁₋₄ alkylenyl, and di-N,N—C₁₋₆ alkylaminoC₁₋₄ alkylenyl;

Y₁ is selected from the group consisting of mono-N—C₁₋₆ alkylamino, di-N,N—C₁₋₆ alkylamino, morpholin-4-yl, piperidin-1-yl, pyrrolidin-1-yl, and 4-C₁₋₄ alkylpiperazin-1-yl;

with the proviso that when R₃ is -Z-Ar′—Y—R₄ and Y is —S—, then R₄ is other than alkyl; with the further proviso that when R₃ is -Z-Ar′—Y—R₄ and Y is —N(R₈)-Q- and R₈ is hydrogen or alkyl and Q is a bond, then R₄ is other than hydrogen or alkyl; with the further proviso that when R₃ is -Z-Ar′—Y—R₄ and Y is —O—, then R₄ is other than hydrogen, alkyl, or haloalkyl; and with the further proviso that when R₃ is -Z-Ar′—X—Y—R₄ and X is —CH₂— and Y is —O—, then R₄ is other than alkyl;

or a pharmaceutically acceptable salt thereof.

For certain embodiments of the compounds of Formulas (I) through (VI), the —NH₂ group can be replaced by an —NH-G group, as shown in the compound of Formula (VII), to form prodrugs. In such embodiments, G is selected from the group consisting of: —C(O)—R′,α-aminoacyl, α-aminoacyl-α-aminoacyl, —C(O)—O—R′, —C(O)—N(R″)R′, —C(═NY′)—R′, —CH(OH)—C(O)—OY′, —CH(OC₁₋₄ alkyl)Y₀, —CH₂Y₁, and —CH(CH₃)Y₁. In some of these embodiments G is —C(O)—R′,α-aminoacyl, α-aminoacyl-α-aminoacyl, or —C(O)—O—R′. Preferably, R′ and R″ are independently selected from the group consisting of C₁₋₁₀ alkyl, C₃₋₇ cycloalkyl, and benzyl, each of which may be unsubstituted or substituted by one or more substitutents selected from the group consisting of halogen, hydroxy, nitro, cyano, carboxy, C₁₋₆ alkyl, C₁₋₄ alkoxy, aryl, heteroaryl, arylC₁₋₄ alkylenyl, heteroarylC₁₋₄ alkylenyl, haloC₁₋₄ alkylenyl, haloC₁₋₄ alkoxy, —O—C(O)—CH₃, —C(O)—O—CH₃, —C(O)—NH₂, —O—CH₂—C(O)—NH₂, —NH₂, and —S(O)₂—NH₂. R″ can also be hydrogen. Preferably, α-aminoacyl is an acyl group derived from an amino acid selected from the group consisting of racemic, D-, and L-amino acids. Preferably, Y′ is selected from the group consisting of hydrogen, C₁₋₆ alkyl, and benzyl. Preferably, Y₀ is selected from the group consisting of C₁₋₆ alkyl, carboxyC₁₋₆ alkylenyl, aminoC₁₋₄ alkylenyl, mono-N—C₁₋₆ alkylaminoC₁₋₄ alkylenyl, and di-N,N—C₁₋₆ alkylaminoC₁₋₄ alkylenyl. Preferably, Y₁ is selected from the group consisting of mono-N—C₁₋₆ alkylamino, di-N,N—C₁₋₆ alkylamino, morpholin-4-yl, piperidin-1-yl, pyrrolidin-1-yl, and 4-C₁₋₄ alkylpiperazin-1-yl.

As used herein, the terms “alkyl,” “alkenyl,” “alkynyl” and the prefix “alk-” are inclusive of both straight chain and branched chain groups and of cyclic groups, e.g., cycloalkyl and cycloalkenyl. Unless otherwise specified, these groups contain from 1 to 20 carbon atoms, with alkenyl groups containing from 2 to 20 carbon atoms, and alkynyl groups containing from 2 to 20 carbon atoms. In some embodiments, these groups have a total of up to 10 carbon atoms, up to 8 carbon atoms, up to 6 carbon atoms, or up to 4 carbon atoms. Cyclic groups can be monocyclic or polycyclic and preferably have from 3 to 10 ring carbon atoms. Exemplary cyclic groups include cyclopropyl, cyclopropylmethyl, cyclopentyl, cyclohexyl, adamantyl, and substituted and unsubstituted bornyl, norbornyl, and norbornenyl.

Unless otherwise specified, “alkylene,” “alkenylene,” and “alkynylene” are the divalent forms of the “alkyl,” “alkenyl,” and “alkynyl” groups defined above. The terms “alkylenyl,” “alkenylenyl,” and “alkynylenyl” are used when “alkylene”, “alkenylene, and “alkynylene”, respectively, are substituted. For example, an arylalkylenyl group comprises an “alkylene” moiety to which an aryl group is attached.

The term “haloalkyl” is inclusive of alkyl groups that are substituted by one or more halogen atoms, including perfluorinated groups. This is also true of other groups that include the prefix “halo-”. Examples of suitable haloalkyl groups are chloromethyl, trifluoromethyl, and the like.

The term “aryl” as used herein includes carbocyclic aromatic rings or ring systems. Examples of aryl groups include phenyl, naphthyl, biphenyl, fluorenyl and indenyl.

The term “heteroatom” refers to the atoms O, S, or N.

The term “heteroaryl” includes aromatic rings or ring systems that contain at least one ring heteroatom (e.g., O, S, N). In some embodiments, the term “heteroaryl” includes a ring or ring system that contains 2 to 12 carbon atoms, 1 to 3 rings, 1 to 4 heteroatoms, and O, S, and/or N as the heteroatoms. Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

The term “heterocyclyl” includes non-aromatic rings or ring systems that contain at least one ring heteroatom (e.g., O, S, N) and includes all of the fully saturated and partially unsaturated derivatives of the above mentioned heteroaryl groups. In some embodiments, the term “heterocyclyl” includes a ring or ring system that contains 2 to 12 carbon atoms, 1 to 3 rings, 1 to 4 heteroatoms, and O, S, and N as the heteroatoms. Exemplary heterocyclic groups include pyrrolidinyl, tetrahydrofuranyl, morpholinyl, thiomorpholinyl, 1,1-dioxothiomorpholinyl, piperidinyl, piperazinyl, thiazolidinyl, imidazolidinyl, isothiazolidinyl, tetrahydropyranyl, quinuclidinyl, homopiperidinyl(azepanyl), 1,4-oxazepanyl, homopiperazinyl(diazepanyl), 1,3-dioxolanyl, aziridinyl, azetidinyl, dihydroisoquinolin-(1H)-yl, octahydroisoquinolin-(1H)-yl, dihydroquinolin-(2H)-yl, octahydroquinolin-(2H)-yl, dihydro-1H-imidazolyl, 3-azabicyclo[3.2.2]non-3-yl, and the like.

The term “heterocyclyl” includes bicylic and tricyclic heterocyclic ring systems. Such ring systems include fused and/or bridged rings and spiro rings. Fused rings can include, in addition to a saturated or partially saturated ring, an aromatic ring, for example, a benzene ring. Spiro rings include two rings joined by one spiro atom and three rings joined by two spiro atoms.

When “heterocyclyl” contains a nitrogen atom, the point of attachment of the heterocyclyl group may be the nitrogen atom.

The terms “arylene,” “heteroarylene,” and “heterocyclylene” are the divalent forms of the “aryl,” “heteroaryl,” and “heterocyclyl” groups defined above. Likewise, “arylenyl,” “heteroarylenyl,” and “heterocyclylenyl” are the divalent forms of the “aryl,” “heteroaryl,” and “heterocyclyl” groups defined above. For example, an alkylarylenyl group comprises an arylene moiety to which an alkyl group is attached.

When a group (or substituent or variable) is present more that once in any Formula described herein, each group (or substituent or variable) is independently selected, whether specifically stated or not. For example, for the formula N(R₈)₂-alkylenyl, each R₈ group is independently selected. In another example, when an R₂ and an R₃ group both contain an R₈ group, each R₈ group is independently selected.

The invention is inclusive of the compounds described herein and salts thereof in any of their pharmaceutically acceptable forms, including isomers such as diastereomers and enantiomers, solvates, polymorphs, prodrugs, and the like. In particular, if a compound is optically active, the invention specifically includes each of the compound's enantiomers as well as racemic mixtures of the enantiomers. It should be understood that the term “compound” includes any or all of such forms, whether explicitly stated or not (although at times, “salts” are explicitly stated).

The term “prodrug” means a compound that can be transformed in vivo to yield an immune response modifying compound in any of the salt, solvated, polymorphic, or isomeric forms described above. The prodrug, itself, may be an immune response modifying compound in any of the salt, solvated, polymorphic, or isomeric forms described above. The transformation may occur by various mechanisms, such as through a chemical (e.g., solvolysis or hydrolysis, for example, in the blood) or enzymatic biotransformation. A discussion of the use of prodrugs is provided by T. Higuchi and W. Stella, “Pro-drugs as Novel Delivery Systems,” Vol. 14 of the A. C. S. Symposium Series, and in Bioreversible Carriers in Drug Design, ed. Edward B. Roche, American Pharmaceutical Association and Pergamon Press, 1987.

Preparation of the Compounds

Compounds of the invention may be synthesized by synthetic routes that include processes analogous to those well known in the chemical arts, particularly in light of the description contained herein. The starting materials are generally available from commercial sources such as Aldrich Chemicals (Milwaukee, Wis., USA) or are readily prepared using methods well known to those skilled in the art (e.g., prepared by methods generally described in Louis F. Fieser and Mary Fieser, Reagents for Organic Synthesis, v. 1-19, Wiley, New York, (1967-1999 ed.); Alan R. Katritsky, Otto Meth-Cohn, Charles W. Rees, Comprehensive Organic Functional Group Transformations, v 1-6, Pergamon Press, Oxford, England, (1995); Barry M. Trost and Ian Fleming, Comprehensive Organic Synthesis, v. 1-8, Pergamon Press, Oxford, England, (1991); or Beilsteins Handbuch der organischen Chemie, 4, Aufl. Ed. Springer-Verlag, Berlin, Germany, including supplements (also available via the Beilstein online database)).

For illustrative purposes, the reaction schemes depicted below provide potential routes for synthesizing the compounds of the present invention as well as key intermediates. For more detailed description of the individual reaction steps, see the EXAMPLES section below. Those skilled in the art will appreciate that other synthetic routes may be used to synthesize the compounds of the invention. Although specific starting materials and reagents are depicted in the reaction schemes and discussed below, other starting materials and reagents can be easily substituted to provide a variety of derivatives and/or reaction conditions. In addition, many of the compounds prepared by the methods described below can be further modified in light of this disclosure using conventional methods well known to those skilled in the art.

Conventional methods and techniques of separation and purification can be used to isolate compounds of the invention, pharmaceutically acceptable salts thereof, as well as various intermediates related thereto. Such techniques may include, for example, all types of chromatography (high performance liquid chromatography (HPLC), column chromatography using common absorbents such as silica gel, and thin layer chromatography), recrystallization, and differential (i.e., liquid-liquid) extraction techniques.

Compounds of the invention can be prepared according to Reaction Scheme I, wherein R, R₂, and n are as defined above; E is a carbon or nitrogen; R_{3b} is -Z-Ar, -Z-Ar′—Y—R₄, -Z-Ar′—X—Y—R₄, or -Z-Ar′—R₅ wherein -Z- is a bond, alkenylene, or alkynylene; Hal is bromo or iodo; R_{3c} is -Z-Ar, -Z-Ar′—Y—R₄, -Z-Ar′—X—Y—R₄, or -Z-Ar′—R₅ wherein -Z- is alkylene; and Ar, Ar′, X, Y, R₄, and R₅ are as defined above. Scheme I begins with a halogenated aniline or halogenated aminopyridine of Formula XV, many of which are commercially available or can be prepared using conventional synthetic methods. In step (1) of Reaction Scheme I, a halogenated aniline or halogenated aminopyridine of Formula XV is treated with the condensation product generated from 2,2-dimethyl-1,3-dioxane-4,6-dione (Meldrum's acid) and triethyl orthoformate to provide an imine of Formula XVI. The reaction is conveniently carried out by adding a solution of a halogenated aniline or halogenated aminopyridine of Formula XV to a heated mixture of Meldrum's acid and triethyl orthoformate and heating the reaction at an elevated temperature such as 55° C.

In step (2) of Reaction Scheme I, an imine of Formula XVI undergoes thermolysis and cyclization to provide a compound of Formula XVII. The reaction is conveniently carried out in a medium such as DOWTHERM A heat transfer fluid at a temperature between 200 and 250° C.

In step (3) of Reaction Scheme I, a compound of Formula XVII is nitrated under conventional nitration conditions to provide a compound of Formula XVIII. The reaction is conveniently carried out by adding nitric acid to a compound of Formula XVII in a suitable solvent such as propionic acid and heating the mixture at an elevated temperature such as 110° C.

In step (4) of Reaction Scheme I, the nitro group of a compound of Formula XVIII is reduced to an amino group. The reaction can be carried out by hydrogenation using a heterogeneous hydrogenation catalyst such as platinum on carbon. The hydrogenation is conveniently carried out in a Parr apparatus in a suitable solvent such as toluene, methanol, acetonitrile, or N,N-dimethylformamide (DMF). The reaction can be carried out at ambient temperature.

In step (5) of Reaction Scheme I, a compound of Formula XIX is reacted with a carboxylic acid or an equivalent thereof to provide a compound of Formula XX. Suitable equivalents to carboxylic acid include acid anhydrides and acid chlorides. The reaction is conveniently carried out by adding the acid chloride to a solution of a compound of Formula XIX in a suitable solvent such as dichloromethane or acetonitrile in the presence of a tertiary amine such as triethylamine, pyridine, or 4-dimethylaminopyridine to afford an amide. The reaction can be carried out at or below ambient temperature.

In step (6) of Reaction Scheme I, an amide of Formula XX is reacted with phosphorus pentasulfide to provide a compound of compound of Formula XXI. The reaction can be carried out by adding phosphorus pentasulfide to a solution or suspension of a compound of Formula XX in a suitable solvent such as pyridine and heating the resulting mixture.

In step (7) of Reaction Scheme I, a compound of Formula XXI is oxidized to provide an N-oxide of Formula XXII using a conventional oxidizing agent capable of forming N-oxides. The reaction is conveniently carried out by adding 3-chloroperoxybenzoic acid to a solution of the compound of Formula XXI in a solvent such dichloromethane or chloroform. The reaction can be carried out at ambient temperature.

In step (8) of Reaction Scheme I, an N-oxide of Formula XXII is aminated to provide a compound of Formula XXIII. Step (8) can be carried out by the activation of an N-oxide of Formula XXII by conversion to an ester and then reacting the ester with an aminating agent. Suitable activating agents include alkyl- or arylsulfonyl chlorides such as benzenesulfonyl chloride, methanesulfonyl chloride, or p-toluenesulfonyl chloride. Suitable aminating agents include ammonia, in the form of ammonium hydroxide, for example, and ammonium salts such as ammonium carbonate, ammonium bicarbonate, and ammonium phosphate. The reaction is conveniently carried out by adding ammonium hydroxide followed by p-toluenesulfonyl chloride to a solution of the N-oxide of Formula XXII in a suitable solvent such as 1,2-dichloroethane at elevated temperature. The reaction may be carried out by adding ammonium hydroxide and p-toluenesulfonyl chloride to the reaction mixture from step (7) without isolating the N-oxide of Formula XXII.

Alternatively step (8) can be carried out by the reaction of a N-oxide of Formula XXII with trichloroacetyl isocyanate followed by hydrolysis of the resulting intermediate to provide a compound of Formula XXIII. The reaction is conveniently carried out in two steps by (i) adding trichloroacetyl isocyanate to a solution of the N-oxide of Formula XXII in a solvent such as dichloromethane and stirring at ambient temperature to provide an isolable amide intermediate. In step (ii), a solution of the intermediate in methanol is treated with a base such as sodium methoxide or ammonium hydroxide at ambient temperature.

Step (9) of Reaction Scheme I can be carried out using known palladium-catalyzed coupling reactions such as the Suzuki coupling and the Heck reaction. For example, a compound of Formula XXIII undergoes Suzuki coupling with a boronic acid of Formula R_{3b}—B(OH)₂, an anhydride thereof, or a boronic acid ester of Formula R_{3b}—B(O-alkyl)₂ to provide a compound of Formula XXIV, which is a subgenus of Formula I wherein R_{3b} is as defined above and Z is a bond or alkenylene. The coupling is carried out by combining a compound of Formula XXIII with a boronic acid or an ester or anhydride thereof in the presence of palladium (II) acetate, triphenylphosphine, and a base such as sodium carbonate in a suitable solvent such as n-propanol. The reaction can be carried out at an elevated temperature, for example, at the reflux temperature. Numerous boronic acids of Formula R_{3b}—B(OH)₂, anhydrides thereof, and boronic acid esters of Formula R_{3b}—B(O-alkyl)₂ are commercially available; others can be readily prepared using known synthetic methods. See, for example, Li, W. et al, J. Org. Chem., 67, 5394-5397 (2002). The product of Formula XXIV or a pharmaceutically acceptable salt thereof can be isolated by conventional methods.

The Heck reaction can also be used in step (9) of Reaction Scheme I to provide compounds of Formula XXIV, wherein R_{3b} is defined as above and -Z- is alkenylene. The Heck reaction is carried out by coupling a compound of Formula XXIII with a compound of the Formula H₂C═C(H)—Ar, H₂C═C(H)—Ar′—Y—R₄, and H₂C═C(H)—Ar′—X—Y—R₄. Several of these vinyl-substituted compounds are commercially available; others can be prepared by known methods. The reaction is conveniently carried out by combining the compound of Formula XXIII and the vinyl-substituted compound in the presence of palladium (II) acetate, triphenylphosphine or tri-ortho-tolylphosphine, and a base such as triethylamine in a suitable solvent such as acetonitrile or toluene. The reaction can be carried out at an elevated temperature such as 100-120 ° C. under an inert atmosphere. Alternatively, a two step route may be utilized in which a compound of Formula XXIII undergoes a palladium catalyzed Stille coupling with a compound of the Formula (alkyl)₃Sn—C(H)═CH₂ to yield an isolable vinyl-substituted compound which may be coupled in a Heck reaction with a compound of the Formula Ar-Halide or Ar′-Halide where Halide is preferably bromide or iodide. The product of Formula XXIV or pharmaceutically acceptable salt thereof can be isolated using conventional methods.

Compounds of Formula XXIV, wherein R_{3b} is defined as above and -Z- is alkynylene, can also be prepared by palladium catalyzed coupling reactions such as the Stille coupling or Sonogashira coupling. These reactions are carried out by coupling a compound of Formula XXIII with a compound of the Formula (alkyl)₃Sn—C≡C—Ar or (alkyl)₃Si—C≡C—Ar.

Compounds of the invention, wherein -Z- is alkylene, can be prepared as shown in step (10) of Reaction Scheme I. In step (10) of Reaction Scheme I, a compound of Formula XXIV, wherein R_{3b} is as defined above and -Z- is alkenylene or alkynylene, is reduced to provide a compound of Formula XXV, which is a subgenus of Formula I. The reduction can be carried out by hydrogenation using a conventional heterogeneous hydrogenation catalyst such as palladium on carbon. The reaction can conveniently be carried out on a Parr apparatus in a suitable solvent such as ethanol, methanol, or mixtures thereof. The product or pharmaceutically acceptable salt thereof can be isolated using conventional methods.

Isomers of aminopyridines of Formula XV are also available and can be used to prepare compounds of Formulas IV, V, and VI according to Reaction Scheme I.

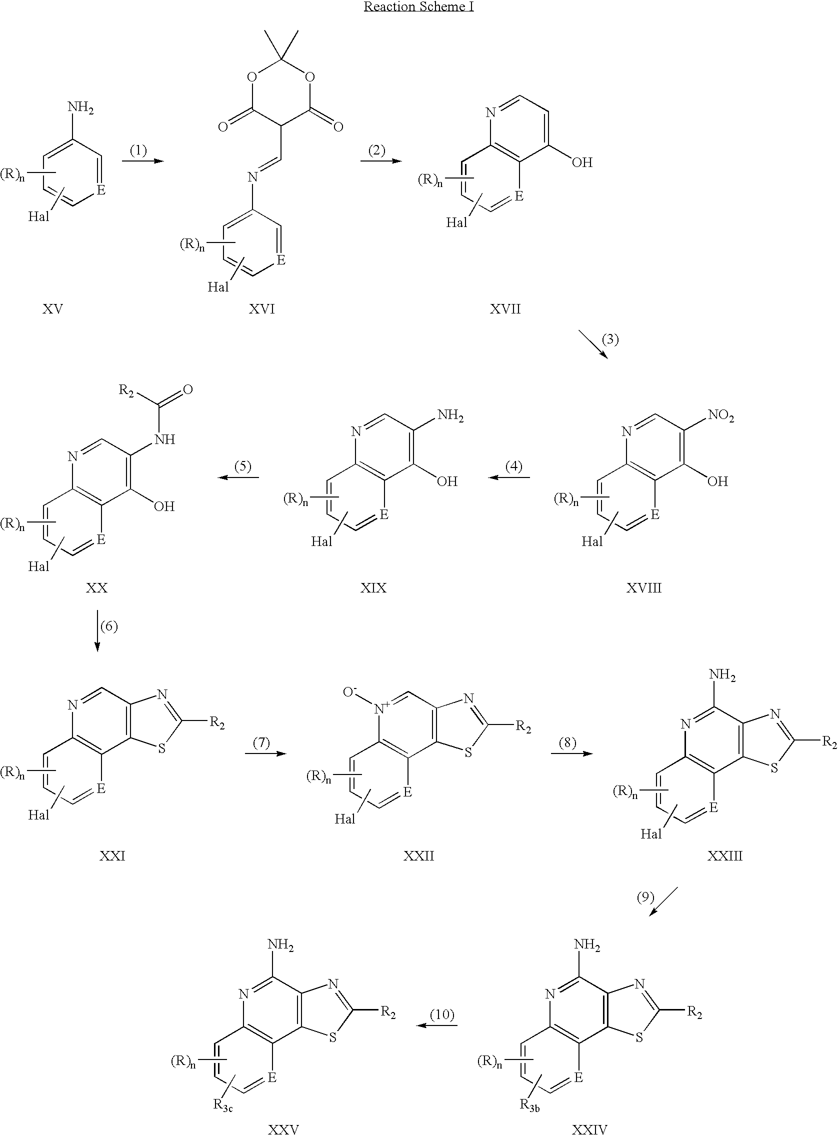

Compounds of the invention can also be prepared according to Reaction Scheme II, wherein R₂ and R₄ are as defined above, E is a carbon or a nitrogen, X′ is a bond or methylene, and Q′ is selected from the group consisting of —C(R₆)—, —S(O)₂—, —C(R₆)—N(R₈)—W—, —S(O)₂—N(R₈)— wherein R₆, R₈, and W are as defined above.

In step (1) of Reaction Scheme II, a compound of Formula XXIIIa, which is a subgenus of Formula XXIII, is coupled with a boronic acid of Formula XXVI to provide a compound of Formula XXVII. The reaction can be carried out as described in step (9) of Reaction Scheme I.

In step (2) of Reaction Scheme II, a compound of Formula XXVII is converted to an amide, sulfonamide, sulfamide, or urea of Formula XXVIII using conventional methods. In step (2), a compound of Formula XXVII can react with an acid chloride of Formula R₄C(O)Cl to provide a compound of Formula XXVIII in which -Q- is —C(O)—. In addition, a compound of Formula XXVII can react with sulfonyl chloride of Formula R₄S(O)₂Cl or a sulfonic anhydride of Formula (R₄S(O)₂)₂O to provide a compound of Formula XXVIII in which -Q- is —S(O)₂—. Numerous acid chlorides of Formula R₄C(O)Cl, sulfonyl chlorides of Formula R₄S(O)₂Cl, and sulfonic anhydrides of Formula (R₄S(O)₂)₂O are commercially available; others can be readily prepared using known synthetic methods. The reaction is conveniently carried out by adding the acid chloride of Formula R₄C(O)Cl, sulfonyl chloride of Formula R₄S(O)₂Cl, or sulfonic anhydride of Formula (R₄S(O)₂)₂O to a solution of the compound of Formula XXVII in a suitable solvent such as chloroform, dichloromethane, DMF, or N,N-dimethylacetamide. Optionally a base such as triethylamine or N,N-diisopropylethylamine can be added. The reaction can be carried out at ambient temperature or a sub-ambient temperature such as 0° C. The product or pharmaceutically acceptable salt thereof can be isolated using conventional methods.

Ureas of Formula XXVIII, where -Q- is —C(O)—N(R₈)— and R₈ is as defined above, can be prepared by reacting a compound of Formula XXVIII with isocyanates of Formula R₄N═C═O or with carbamoyl chlorides of Formula R₄N—(R₈)—C(O)Cl. Numerous isocyanates of Formula R₄N═C═O and carbamoyl chlorides of Formula R₄N—(R₈)—C(O)Cl are commercially available; others can be readily prepared using known synthetic methods. The reaction can be conveniently carried out by adding the isocyanate of Formula R₄N═C═O or carbamoyl chloride of Formula R₄N—(R₈)—C(O)Cl to a solution of the compound of Formula XXVII in a suitable solvent such as DMF, chloroform, or N,N-dimethylacetamide. Optionally a base such as triethylamine or N,N-diisopropylethylamine can be added. The reaction can be carried out at ambient temperature or a sub-ambient temperature such as 0° C. Alternatively, a compound of Formula XXVII can be treated with an isocyanate of Formula R₄(CO)N═C═O, a thioisocyanate of Formula R₄N═C═S, or a sulfonyl isocyanate of Formula R₄S(O)₂N═C═O to provide a compound of Formula XXVIII, where -Q- is —C(O)—N(R₈)—(CO)—, —C(S)—N(R₈)—, or —C(O)—N(R₈)—S(O)₂—, respectively. The product or pharmaceutically acceptable salt thereof can be isolated using conventional methods.

Sulfamides of Formula XXVIII, where -Q- is —S(O)₂—N(R₈)—, can be prepared by reacting a compound of Formula XXVII with sulfuryl chloride to generate a sulfamoyl chloride in situ, and then reacting the sulfamoyl chloride with an amine of formula HN(R₈)R₄. Alternatively, sulfamides of Formula XXVIII can be prepared by reacting a compound of Formula XXVII with a sulfamoyl chloride of formula R₄(R₈)N—S(O)₂Cl. The product or a pharmaceutically acceptable salt thereof can be isolated using conventional methods. Many amines of Formula HN(R₈)R₄ and some sulfamoyl chlorides of formula R₄(R₈)N—S(O)₂Cl are commercially available; others can be prepared using known synthetic methods. The product or pharmaceutically acceptable salt thereof can be isolated using conventional methods.

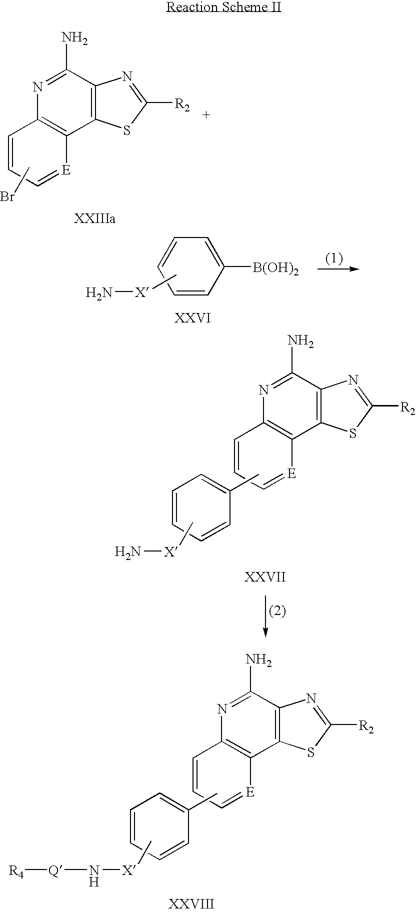

Compounds of the invention can also be prepared using the synthetic routes described in the EXAMPLES below.

Prodrugs can be prepared in a variety of ways. For example, a compound wherein R₂ is hydroxyalkylenyl can be converted into a prodrug wherein R₂ is, for example, -alkylenyl-O—C(R₆)—R₄, -alkylenyl-O—C(R₆)—O—R₄, or -alkylenyl-O—C(R₆)—N(R₈)—R₄, wherein R₄, R₆, and R₈ are as defined above, using methods known to one skilled in the art. In addition, a compound wherein Ar is substituted by a hydroxyalkylenyl group may also be converted to an ester, an ether, a carbonate, or a carbamate. For any of these compounds containing an alcohol functional group, a prodrug can be formed by the replacement of the hydrogen atom of the alcohol group with a group such as C₁₋₆ alkanoyloxymethyl, 1-(C₁₋₆ alkanoyloxy)ethyl, 1-methyl-1-(C₁₋₆ alkanoyloxy)ethyl, C₁₋₆ alkoxycarbonyloxymethyl, N—(C₁₋₆ alkoxycarbonyl)aminomethyl, succinoyl, C₁₋₆ alkanoyl, α-aminoC₁₋₄ alkanoyl, arylacyl, —P(O)(OH)₂, —P(O)(O—C₁₋₆ alkyl)₂, C₁₋₆ alkoxycarbonyl, C₁₋₆ alkylcarbamoyl, and α-aminoacyl or α-aminoacyl-α-aminoacyl, where each α-aminoacyl group is independently selected from racemic, D-, and L-amino acids. For compounds containing an alcohol functional group, particularly useful prodrugs are esters made from carboxylic acids containing one to six carbon atoms, unsubstituted or substituted benzoic acid esters, or esters made from racemic, D-, or L-amino acids.

Prodrugs can also be made from a compound containing an amino group by conversion of the amino group to a functional group such as an amide, carbamate, urea, amidine, or another hydrolysable group using conventional methods. A prodrug of this type can be made by the replacement of a hydrogen atom in an amino group, particularly the amino group at the 4-position, with a group such as —C(O)—R′, α-aminoacyl, α-aminoacyl-α-aminoacyl, —C(O)—O—R′, —C(O)—N(R″)—R′, —C(═NY′)—R′, —CH(OH)—C(O)—OY′, —CH(OC₁₋₄ alkyl)Y₀, —CH₂Y₁, or —CH(CH₃)Y₁; wherein R′ and R″ are each independently C₁₋₁₀ alkyl, C₃₋₇ cycloalkyl, or benzyl, each of which may be unsubstituted or substituted by one or more substituents selected from the group consisting of halogen, hydroxy, nitro, cyano, carboxy, C₁₋₆ alkyl, C₁₋₄ alkoxy, aryl, heteroaryl, arylC₁₋₄ alkylenyl, heteroarylC₁₋₄ alkylenyl, haloC₁₋₄ alkylenyl, haloC₁₋₄ alkoxy, —O—C(O)—CH₃, —C(O)—O—CH₃, —C(O)—NH₂, —O—CH₂—C(O)—NH₂, —NH₂, and —S(O)₂—NH₂, with the proviso that R″ can also be hydrogen; each α-aminoacyl group is independently selected from racemic, D-, and L-amino acids; Y′ is hydrogen, C₁₋₆ alkyl, or benzyl; Y₀ is C₁₋₆ alkyl, carboxyC₁₋₆ alkylenyl, aminoC₁₋₄ alkylenyl, mono-N—C₁₋₆ alkylaminoC₁₋₄ alkylenyl, or di-N,N—C₁₋₆ alkylaminoC₁₋₄ alkylenyl; and Y₁ is mono-N—C₁₋₆ alkylamino, di-N,N-C₁₋₆ alkylamino, morpholin-4-yl, piperidin-1-yl, pyrrolidin-1-yl, or 4-C₁₋₄ alkylpiperazin-1-yl.

Pharmaceutical Compositions and Biological Activity

Pharmaceutical compositions of the invention contain a therapeutically effective amount of a compound or salt of the invention as described above in combination with a pharmaceutically acceptable carrier.

The terms “a therapeutically effective amount” and “effective amount” mean an amount of the compound or salt sufficient to induce a therapeutic or prophylactic effect, such as cytokine induction, immunomodulation, antitumor activity, and/or antiviral activity. Although the exact amount of active compound or salt used in a pharmaceutical composition of the invention will vary according to factors known to those of skill in the art, such as the physical and chemical nature of the compound or salt, the nature of the carrier, and the intended dosing regimen, it is anticipated that the compositions of the invention will contain sufficient active ingredient to provide a dose of about 100 nanograms per kilogram (ng/kg) to about 50 milligrams per kilogram (mg/kg), preferably about 10 micrograms per kilogram (μg/kg) to about 5 mg/kg, of the compound or salt to the subject. A variety of dosage forms may be used, such as tablets, lozenges, capsules, parenteral formulations, syrups, creams, ointments, aerosol formulations, transdermal patches, transmucosal patches and the like.

The compounds or salts of the invention can be administered as the single therapeutic agent in the treatment regimen, or the compounds or salts of the invention may be administered in combination with one another or with other active agents, including additional immune response modifiers, antivirals, antibiotics, antibodies, proteins, peptides, oligonucleotides, etc.

Compounds or salts of the invention have been shown to induce, and certain compounds or salts of the invention may inhibit, the production of certain cytokines in experiments performed according to the tests set forth below. These results indicate that the compounds or salts are useful as immune response modifiers that can modulate the immune response in a number of different ways, rendering them useful in the treatment of a variety of disorders.

Cytokines whose production may be induced by the administration of compounds or salts of the invention generally include interferon-α (IFN-α) and/or tumor necrosis factor-α (TNF-α) as well as certain interleukins (IL). Cytokines whose biosynthesis may be induced by compounds or salts of the invention include IFN-α, TNF-α, IL-1, IL-6, IL-10 and IL-12, and a variety of other cytokines. Among other effects, these and other cytokines can inhibit virus production and tumor cell growth, making the compounds or salts useful in the treatment of viral diseases and neoplastic diseases. Accordingly, the invention provides a method of inducing cytokine biosynthesis in an animal comprising administering an effective amount of a compound or salt or composition of the invention to the animal. The animal to which the compound or salt or composition is administered for induction of cytokine biosynthesis may have a disease as described infra, for example a viral disease or a neoplastic disease, and administration of the compound or salt may provide therapeutic treatment. Alternatively, the compound or salt may be administered to the animal prior to the animal acquiring the disease so that administration of the compound or salt may provide a prophylactic treatment.

In addition to the ability to induce the production of cytokines, compounds or salts of the invention can affect other aspects of the innate immune response. For example, natural killer cell activity may be stimulated, an effect that may be due to cytokine induction. The compounds or salts may also activate macrophages, which in turn stimulate secretion of nitric oxide and the production of additional cytokines. Further, the compounds or salts may cause proliferation and differentiation of B-lymphocytes.

Compounds or salts of the invention can also have an effect on the acquired immune response. For example, the production of the T helper type 1 (T_{H}1) cytokine IFN-γ may be induced indirectly and the production of the T helper type 2 (T_{H}2) cytokines IL-4, IL-5 and IL-13 may be inhibited upon administration of the compounds or salts.

Other cytokines whose production may be inhibited by the administration of compounds or salts of the invention include tumor necrosis factor-α (TNF-α). Among other effects, inhibition of TNF-α production can provide prophylaxis or therapeutic treatment of TNF-α mediated diseases in animals, making the compounds or salt useful in the treatment of, for example, autoimmune diseases. Accordingly, the invention provides a method of inhibiting TNF-α biosynthesis in an animal comprising administering an effective amount of a compound or salt or composition of the invention to the animal. The animal to which the compound or salt or composition is administered for inhibition of TNF-α biosynthesis may have a disease as described infra, for example an autoimmune disease, and administration of the compound or salt may provide therapeutic treatment. Alternatively, the compound or salt may be administered to the animal prior to the animal acquiring the disease so that administration of the compound or salt may provide a prophylactic treatment.

Whether for prophylaxis or therapeutic treatment of a disease, and whether for effecting innate or acquired immunity, the compound or salt or composition may be administered alone or in combination with one or more active components as in, for example, a vaccine adjuvant. When administered with other components, the compound or salt and other component or components may be administered separately; together but independently such as in a solution; or together and associated with one another such as (a) covalently linked or (b) non-covalently associated, e.g., in a colloidal suspension.

Conditions for which compounds or salts identified herein may be used as treatments include, but are not limited to:

(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);

(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;

(c) other infectious diseases, such chlamydia, fungal diseases including but not limited to candidiasis, aspergillosis, histoplasmosis, cryptococcal meningitis, or parasitic diseases including but not limited to malaria, pneumocystis carnii pneumonia, leishmaniasis, cryptosporidiosis, toxoplasmosis, and trypanosome infection;

(d) neoplastic diseases, such as intraepithelial neoplasias, cervical dysplasia, actinic keratosis, basal cell carcinoma, squamous cell carcinoma, renal cell carcinoma, Kaposi's sarcoma, melanoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers;

(e) T_{H}2-mediated, atopic diseases, such as atopic dermatitis or eczema, eosinophilia, asthma, allergy, allergic rhinitis, and Ommen's syndrome;

(f) certain autoimmune diseases such as systemic lupus erythematosus, essential thrombocythaemia, multiple sclerosis, discoid lupus, alopecia areata; and

(g) diseases associated with wound repair such as, for example, inhibition of keloid formation and other types of scarring (e.g., enhancing wound healing, including chronic wounds).

Additionally, a compound or salt of the present invention may be useful as a vaccine adjuvant for use in conjunction with any material that raises either humoral and/or cell mediated immune response, such as, for example, live viral, bacterial, or parasitic immunogens; inactivated viral, tumor-derived, protozoal, organism-derived, fungal, or bacterial immunogens; toxoids; toxins; self-antigens; polysaccharides; proteins; glycoproteins; peptides; cellular vaccines; DNA vaccines; autologous vaccines; recombinant proteins; and the like, for use in connection with, for example, BCG, cholera, plague, typhoid, hepatitis A, hepatitis B, hepatitis C, influenza A, influenza B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, yellow fever, and Alzheimer's Disease.

Compounds or salts of the present invention may be particularly helpful in individuals having compromised immune function. For example, compounds or salts may be used for treating the opportunistic infections and tumors that occur after suppression of cell mediated immunity in, for example, transplant patients, cancer patients and HIV patients.

Thus, one or more of the above diseases or types of diseases, for example, a viral disease or a neoplastic disease may be treated in an animal in need thereof (having the disease) by administering a therapeutically effective amount of a compound or salt of the invention to the animal.

An amount of a compound or salt effective to induce or inhibit cytokine biosynthesis is an amount sufficient to cause one or more cell types, such as monocytes, macrophages, dendritic cells and B-cells to produce an amount of one or more cytokines such as, for example, IFN-α, TNF-α, IL-1, IL-6, IL-10 and IL-12 that is increased (induced) or decreased (inhibited) over a background level of such cytokines. The precise amount will vary according to factors known in the art but is expected to be a dose of about 100 ng/kg to about 50 mg/kg, preferably about 10 μg/kg to about 5 mg/kg. The invention also provides a method of treating a viral infection in an animal and a method of treating a neoplastic disease in an animal comprising administering an effective amount of a compound or salt or composition of the invention to the animal. An amount effective to treat or inhibit a viral infection is an amount that will cause a reduction in one or more of the manifestations of viral infection, such as viral lesions, viral load, rate of virus production, and mortality as compared to untreated control animals. The precise amount that is effective for such treatment will vary according to factors known in the art but is expected to be a dose of about 100 ng/kg to about 50 mg/kg, preferably about 10 μg/kg to about 5 mg/kg. An amount of a compound or salt effective to treat a neoplastic condition is an amount that will cause a reduction in tumor size or in the number of tumor foci. Again, the precise amount will vary according to factors known in the art but is expected to be a dose of about 100 ng/kg to about 50 mg/kg, preferably about 10 μg/kg to about 5 mg/kg.

In addition to the formulations and uses described specifically herein, other formulations, uses, and administration devices suitable for compounds of the present invention are described in, for example, International Publication Nos. WO 03/077944 and WO 02/036592, U.S. Pat. No. 6,245,776, and U.S. Publication Nos. 2003/0139364, 2003/185835, 2004/0258698, 2004/0265351, 2004/076633, and 2005/0009858.

### EXAMPLES

Objects and advantages of this invention are further illustrated by the following examples, but the particular materials and amounts thereof recited in these examples, as well as other conditions and details, should not be construed to unduly limit this invention.

### Example 1

### [3-(4-Amino-2-propylthiazolo[4,5-c]quinolin-7-yl)phenyl]methanol

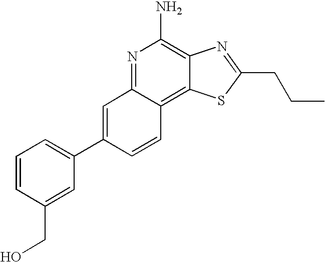 Part A

A mixture of triethyl orthoformate (154 grams (g), 1.04 moles (mol)) and Meldrum's acid (142 g, 0.983 mol) was heated to 55° C. for 4 hours (h). After cooling to 50° C., a solution of 3-bromoaniline (162.6 g, 0.945 mol) in ethanol (300 mL) was added such that the temperature of the reaction was maintained between 50-55° C. After half of the 3-bromoaniline had been added, stirring became difficult due to the formation of solids, so more ethanol (1 liter (L)) was added to facilitate stirring. Upon complete addition, the reaction was cooled to room temperature (rt), and the solids were collected by filtration. The filter cake was washed with ice cold ethanol until the washings were nearly colorless, and the product was dried at 65° C. under vacuum to afford 287 g of 5-[(3-bromophenylamino)methylene]-2,2-dimethyl-[1,3]dioxane-4,6-dione as an off-white solid. ¹H NMR (300 MHz, CDCl₃) δ 11.19 (brd, J=12.8 Hz, 1H), 8.60 (d, J=14.0 Hz, 1H), 7.44-7.38 (m, 2H), 7.30 (t, J=8.0 Hz, 1H), 7.18 (ddd, J=8.0, 2.2, 0.9 Hz, 1H), 1.75 (s, 6H).

Part B

7-Bromoquinolin-4-ol was prepared in accordance with the literature procedure (D. Dibyendu et al., J. Med. Chem., 41, 4918-4926 (1998)) or by thermolysis of 5-[(3-bromophenylamino)methylene]-2,2-dimethyl-[1,3]dioxane-4,6-dione in DOWTHERM A heat transfer fluid and had the following spectral properties: ¹H NMR (300 MHz, d₆-DMSO) δ 11.70 (brs, 1H), 8.00 (d, J=8.7 Hz, 1H), 7.92 (d, J=7.5 Hz, 1H), 7.74 (d, J=1.9 Hz, 1H), 7.44 (dd, J=8.7, 1.9 Hz, 1H), 6.05 (d, J=7.5 Hz, 1H).

Part C

A stirred suspension of 7-bromoquinolin-4-ol (162 g, 0.723 mol) in propionic acid (1500 mL) was brought to 110° C. 70% Nitric acid (85 g) was added dropwise over 1 h such that the temperature was maintained between 110-115° C. After half of the nitric acid had been added, stirring became difficult due to the formation of solids and an additional 200 mL of propionic acid was added. Upon complete addition, the reaction was stirred for 1 h at 110° C., cooled to room temperature, and the solid was collected by filtration. The filter cake was washed with ice cold ethanol until the washings were nearly colorless (800 mL), and the product was dried at 60° C. under vacuum to afford 152 g of 7-bromo-3-nitro-quinolin-4-ol as a pale yellow solid. ¹H NMR (300 MHz, d₆-DMSO) δ 13.0 (brs, 1H), 9.22 (s, 1H), 8.15 (d, J=8.4 Hz, 1H), 7.90 (d, J=1.6 Hz, 1H), 7.66 (dd, J=8.7, 1.9 Hz, 1H).

Part D

A mixture of 7-bromo-3-nitroquinolin-4-ol (27.03 g, 100.5 mmol) and 5% platinum on carbon (2.70 g) in N,N-dimethylformamide (DMF, 270 mL) was hydrogenated on a Parr apparatus. The mixture was filtered through CELITE filter agent, which was washed with DMF. The filtrate was cooled to 0° C. and acidified with hydrogen chloride gas, resulting in the formation of a reddish-brown solid. The solid was filtered, washed with acetone, and dried to yield 25.68 g of 3-amino-7-bromoquinolin-4-ol hydrochloride as a tan solid.

Part E

To a solution of the crude 3-amino-7-bromoquinolin-4-ol hydrochloride (prepared as described in part D, 11.98 g, 43.5 mmol) and triethylamine (12.1 mL, 86.9 mmol) in dichloromethane (175 mL) at 0° C. was added butyryl chloride (4.5 mL, 43.5 mmol). The solution was stirred for 10 min at 0° C. then stirred at ambient temperature overnight. A solid was isolated by filtration and washed with dichloromethane. The solid was slurried with water (60 mL), isolated by filtration, and dried overnight at 60° C. under vacuum to yield 8.94 g of N-(7-bromo-4-hydroxyquinolin-3-yl)butanamide as a light red solid.

Part F

A mixture of N-(7-bromo-4-hydroxyquinolin-3-yl)butanamide (17.62 g, 57.0 mmol), phosphorus pentasulfide (12.67 g, 28.5 mmol), and pyridine (175 mL) was heated at reflux for 2 hours to afford a homogeneous solution. The solution allowed to cool to room temperature (rt) and the excess phosphorus pentasulfide was quenched slowly with 10% aqueous sodium carbonate. The reaction mixture was concentrated under reduced pressure to 100 mL and transferred to a separatory funnel containing water (100 mL). The mixture was extracted with dichloromethane (250 mL, then 100 mL). The combined organic layers were washed with 0.1 M aqueous hydrochloric acid, dried with magnesium sulfate, filtered, and concentrated under reduced pressure to afford a brownish-yellow solid that was concentrated once from heptane (100 mL). The material was boiled in heptane (175 mL) and filtered. The filtrate was allowed to cool to ambient temperature to afford a yellow solid that was isolated by filtration, washed with cold heptane, and dried to yield 12.20 g of 7-bromo-2-propylthiazolo[4,5-c]quinoline as a light yellow solid.

Part G

3-Chloroperoxybenzoic acid (m-CPBA, 65% pure, 9.28 g, 35.0 mmol) was added in small portions to a solution of 7-bromo-2-propylthiazolo[4,5-c]quinoline (7.16 g, 23.3 mmol) in dichloromethane (115 mL) at ambient temperature. The reaction was stirred for 3 hours, then was transferred to a separatory funnel and washed with 10% aqueous sodium carbonate (2×50 mL). The aqueous layer was back-extracted with dichloromethane (50 mL). The combined organic layers were washed with water (75 mL), dried over magnesium sulfate, filtered, and concentrated under reduced pressure to yield 7.31 g of 7-bromo-2-propylthiazolo[4,5-c]quinoline 5-oxide as a light yellow solid.

Part H

Trichloroacetyl isocyanate (2.70 mL, 22.4 mmol) was added in one portion to a light orange solution of 7-bromo-2-propylthiazolo[4,5-c]quinoline 5-oxide (6.02 g, 18.6 mmol) in dichloromethane (120 mL) at 0° C., causing a color change to red. The reaction was allowed to warm to ambient temperature and was stirred overnight, then was concentrated under reduced pressure to yield 9.46 g (109%) of crude N-(7-bromo-2-propylthiazolo[4,5-c]quinolin-4-yl)-2,2,2-trichloroacetamide.

Part I

Sodium methoxide (25 wt. % solution in methanol, 14.1 mL, 65.2 mmol) was added to a mixture of the crude N-(7-bromo-2-propylthiazolo[4,5-c]quinolin-4-yl)-2,2,2-trichloroacetamide from Part H in methanol (120 mL) at rt, resulting in a solution from which a solid began to precipitate. After 2 hours (h), the mixture was concentrated under reduced pressure. The resulting solid was suspended in methanol (˜50 mL) and was isolated by filtration. The solid was washed with methanol and dried to yield 4.89 g of 7-bromo-2-propylthiazolo[4,5-c]quinolin-4-amine as a light yellow solid, mp 160-163° C. ¹H NMR (300 MHz, d₆-DMSO) δ 7.74 (d, J=1.9 Hz, 1H), 7.72 (d, J=7.8 Hz, 1H), 7.35 (dd, J=8.4, 1.9 Hz, 1H), 7.12 (s, 2H), 3.12 (t, J=7.8 Hz, 2H), 1.83 (sextet, J=7.2 Hz, 2H), 0.99 (t, J=7.5 Hz, 3H); ¹³C NMR (75 MHz, d₆-DMSO) δ 171.2, 152.9, 146.0, 138.9, 137.6, 127.6, 126.5, 124.7, 121.5, 117.8, 35.1, 22.8, 13.5; Anal. calcd for C₁₃H₁₂N₃SBr: C, 48.46; H, 3.75; N, 13.04. Found: C, 48.24; H, 3.51; N, 12.89.

Part J

A solution of 7-bromo-2-propylthiazolo[4,5-c]quinolin-4-amine prepared as described in Part I, 1.17 g, 3.63 mmol), 3-(hydroxymethyl)benzene boronic acid (0.66 g, 4.36 mmol), triphenylphosphine (28.6 mg, 0.11 mmol), water (3.5 mL), and 2 M Na₂CO₃ (2.2 mL, 4.4 mmol) in 1-propanol (25 mL) was degassed and placed under a nitrogen atmosphere. To the solution was added a solution of palladium acetate (8.1 mg, 0.036 mmol) in warm toluene (0.25 mL). The reaction solution was degassed and placed under a nitrogen atmosphere again. The solution was heated at 100° C. for 18.5 h, then was allowed to cool to rt. The 1-propanol was removed under reduced pressure and the remaining liquid was diluted with dichloromethane (150 mL), washed with 2 M Na₂CO₃ (50 mL) and brine (50 mL), dried over MgSO₄, filtered, and concentrated to yield a yellow solid. The solid was purified on a HORIZON High-Performance Flash Chromatography (HPFC) instrument (available from Biotage, Inc, Charlottesville, Va., USA) (silica gel, gradient elution with 0-25% CMA/chloroform where CMA is a solution comprised of 80% chloroform, 18% methanol, and 2% concentrated ammonium hydroxide) to yield a white solid that was recrystallized from boiling acetonitrile (50 mL). After drying at 60° C. under vacuum, [3-(4-amino-2-propylthiazolo[4,5-c]quinolin-7-yl)phenyl]methanol was isolated as a white solid (0.34 g), mp 186-188° C. ¹H NMR (300 MHz, d₆-DMSO) δ 7.84 (d, J=8.1 Hz, 1H), 7.83 (d, J=1.9 Hz, 1H), 7.71 (s, 1H), 7.62 (d, J=7.8 Hz, 1H), 7.55 (dd, J=8.4, 1.8 Hz, 1H), 7.44 (t, J=7.8 Hz, 1H), 7.35 (d, J=7.8 Hz, 1H), 6.92 (s, 2H), 5.25 (t, J=6.0 Hz, 1H), 4.59 (d, J=5.9 Hz, 2H), 3.15 (t, J=7.8 Hz, 2H), 1.88 (sextet, J=7.2 Hz, 2H), 1.02 (t, J=7.5 Hz, 3H); ¹³C NMR (75 MHz, d₆-DMSO) δ 170.6, 152.4, 145.2, 143.3, 140.5, 139.7, 138.9, 137.5, 128.7, 125.7, 125.2, 125.1, 124.9, 123.3, 121.0, 118.0, 62.9, 35.1, 22.7, 13.4; Anal. Calcd for C₂₀H₁₉N₃O_{S}: C, 68.74; H, 5.48; N, 12.02. Found: C, 68.49; H, 5.42; N, 11.93.

### Example 2

### N-[3-(4-Amino-2-propylthiazolo[4,5-c]quinolin-7-yl)phenyl]methanesulfonamide

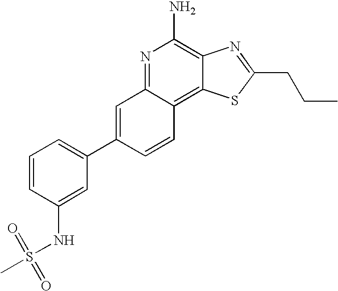

The general method of Part J of Example 1 was followed to couple 7-bromo-2-propylthiazolo[4,5-c]quinolin-4-amine prepared as described in Parts A-I of Example 1, 1.17 g, 3.63 mmol) with 3-(methanesulfonylamino)phenyl boronic acid (0.94 g, 4.36 mmol). The reaction was complete in 3.25 hours. The crude product was isolated and then purified by HPFC (silica gel, gradient elution with 0-20% CMA/chloroform) to provide 0.76 g of a pale yellow solid. The product was further purified by recrystallization from boiling acetonitrile. After drying at 60° C. under vacuum, N-[3-(4-amino-2-propylthiazolo[4,5-c]quinolin-7-yl)phenyl]methanesulfonamide was isolated as yellow needles (0.49 g), mp 228-231° C. ¹H NMR (300 MHz, d₆-DMSO) δ 9.86 (s, 1H), 7.87 (d, J=8.4 Hz, 1H), 7.80 (d, J=6.0 Hz, 1H), 7.59 (m, 1H), 7.53-7.42 (m, 3H), 7.23 (ddd, J=8.2, 2.5, 1.9 Hz, 1H), 6.94 (s, 2H), 3.15 (t, J=7.8 Hz, 2H), 3.07 (s, 3H), 1.88 (sextet, J=7.2 Hz, 2H), 1.02 (t, J=7.5 Hz, 3H); ¹³C NMR (75 MHz, d₆-DMSO) δ 170.8, 152.5, 145.2, 141.1, 139.9, 139.1, 138.9, 137.6, 130.0, 125.4, 123.4, 122.3, 120.8, 118.8, 118.2, 118.0, 39.3, 35.2, 22.8, 13.5; Anal. calcd for C₂₀H₂₀N₄O₂S₂: C, 58.23; H, 4.89; N, 13.58. Found: C, 58.10; H, 4.65; N, 13.43.

### Example 3

### [2-(4-Amino-2-propylthiazolo[4,5-c]quinolin-7-yl)phenyl]methanol

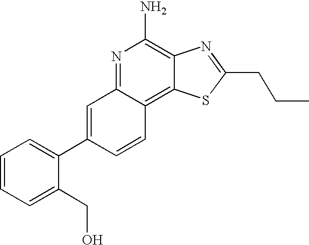

A solution of 7-bromo-2-propylthiazolo[4,5-c]quinolin-4-amine (prepared as described in Parts A-I of Example 1, 1.00 g, 3.10 mmol), 2-(hydroxymethyl)benzene boronic acid (0.57 g, 3.72 mmol), palladium acetate (7 mg, 0.03 mmol), triphenylphosphine (25 mg, 0.093 mmol), water (2 mL), and 2 M Na₂CO₃ (2.0 mL, 4.0 mmol) in 1-propanol (20 mL) was degassed and placed under a nitrogen atmosphere. The light yellow solution was heated at 100° C. for 20 h, then was allowed to cool to rt. The 1-propanol was removed under reduced pressure and the remaining liquid was diluted with dichloromethane (150 mL) and washed with 2 M Na₂CO₃ (50 mL) and brine (50 mL), dried over MgSO₄, filtered, and concentrated to yield a yellow solid. The solid was purified by HPFC (silica gel, gradient elution with 0-25% CMA/chloroform) to yield a white solid that was recrystallized from boiling 2-butanone (60 mL). After drying at 60° C. under vacuum, [2-(4-amino-2-propylthiazolo[4,5-c]quinolin-7-yl)phenyl]methanol was isolated as a white solid (0.615 g), mp 216-219° C. ¹H NMR (300 MHz, d₆-DMSO) δ 7.82 (d, J=8.1 Hz, 1H), 7.61-7.58 (m, 2H), 7.43-7.25 (m, 4H), 6.93 (s, 2H), 5.15 (t, J=5.3 Hz, 1H), 4.45 (d, J=5.3 Hz, 2H), 3.16 (t, J=7.8 Hz, 2H), 1.89 (sextet, J=7.2 Hz, 2H), 1.02 (t, J=7.5 Hz, 3H); ¹³C NMR (75 MHz, d₆-DMSO) δ 170.6, 152.5, 144.6, 140.8, 139.9, 139.3, 138.9, 137.6, 129.3, 128.2, 127.4, 126.9, 125.8, 124.4, 123.4, 117.7, 60.8, 35.2, 22.8, 13.4; Anal. calcd for C₂₀H₁₉N₃O_{S}.0.5 H₂O: C, 67.06; H, 5.62; N, 11.63. Found: C, 66.92; H, 5.24; N, 11.49.

### Example 4

### 7-[3-(Morpholin-4-ylcarbonyl)phenyl]-2-propylthiazolo[4,5-c]quinolin-4-amine

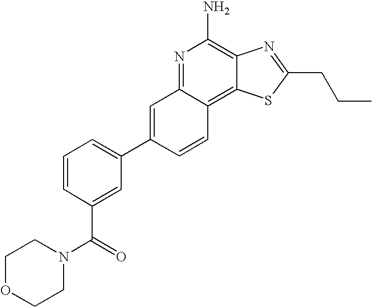

7-Bromo-2-propylthiazolo[4,5-c]quinolin-4-amine (prepared as described in Parts A-I of Example 1, 1.00 g, 3.10 mmol) was reacted with 3-(morpholin-4-ylcarbonyl)phenylboronic acid (0.75 g, 3.72 mmol) according to the method described in Example 3. After the workup and purification by HPFC (silica gel, gradient elution with 0-25% CMA/chloroform), the product was recrystallized from toluene (40 mL) and dried at 60° C. in a vacuum to provide 7-[3-(morpholin-4-ylcarbonyl)phenyl]-2-propylthiazolo[4,5-c]quinolin-4-amine as a light yellow solid (0.87 g, 65%), mp 190-193° C. ¹H NMR (300 MHz, d₆-DMSO) δ 7.88-7.84 (m, 3H), 7.76 (t, J=1.3 Hz, 1H), 7.59 (dd, J=8.2, 1.8 Hz, 1H), 7.57 (t, J=7.5 Hz, 1H), 7.42 (dt, J=7.8, 1.3 Hz, 1H), 6.94 (s, 2H), 3.75-3.40 (m, 8H), 3.15 (t, J=7.8 Hz, 2H), 1.87 (sextet, J=7.2 Hz, 2H), 1.02 (t, J=7.5 Hz, 3H); ¹³C NMR (75 MHz, d₆-DMSO) δ 170.8, 168.8, 152.5, 145.2, 140.1, 139.6, 138.8, 137.7, 136.4, 129.2, 128.8, 128.2, 128.0, 126.2, 125.4, 125.3, 123.6, 121.0, 118.3, 66.0, 35.2, 22.8, 13.5; Anal. calcd for C₂₄H₂₄N₄O₂S.0.12 C₇H₈: C, 67.26; H, 5.67; N, 12.63. Found: C, 67.50; H, 5.91; N, 12.55.

### Examples 5-41

7-Bromo-2-(2-methoxyethyl)thiazolo[4,5-c]quinolin-4-amine, which was used as a substrate in Examples 27-41, was prepared from 3-amino-7-bromoquinolin-4-ol hydrochloride as described in the following paragraphs.

Part A

The preparation of 3-amino-7-bromoquinolin-4-ol hydrochloride was described in Parts A-D of Example 1. The general method described in Part E of Example 1 was followed and 3-methoxypropionyl chloride (8.50 mL, 78.0 mmol) was added in lieu of butyryl chloride to a solution of 3-amino-7-bromoquinolin-4-ol hydrochloride (21.48 g, 77.96 mmol) and triethylamine (21.7 mL, 156 mmol) in dichloromethane (235 mL). The crude product was isolated from the reaction mixture by filtration, slurried in water (2×250 mL), filtered, washed with diethyl ether, and dried overnight at 60° C. in a vacuum oven to yield 23.3 g of N-(7-bromo-4-hydroxyquinolin-3-yl)-3-methoxypropanamide which was contaminated with triethylamine salts. Analysis by ¹H NMR indicated that the mixture contained 17.75 g of the desired N-(7-bromo-4-hydroxyquinolin-3-yl)-3-methoxypropanamide.

Part B

A modification of the general method described in Part F of Example 1 was followed using the N-(7-bromo-4-hydroxyquinolin-3-yl)-3-methoxypropanamide (17.75 g, 54.6 mmol) prepared above in Part A as the starting material. The reaction yielded 11.80 g of 7-bromo-2-(2-methoxyethyl)thiazolo[4,5-c]quinoline as a light yellow solid.

Part C

The general methods described in Part G and H of Example 1, were used to convert 7-bromo-2-(2-methoxyethyl)thiazolo[4,5-c]quinoline (11.78 g, 36.47 mmol) to 15.82 g of N-[7-bromo-2-(2-methoxyethyl)thiazolo[4,5-c]quinolin-4-yl]-2,2,2-trichloroacetamide.

Part D

The general method described in part I of Example 1 was followed using N-[7-bromo-2-(2-methoxyethyl)thiazolo[4,5-c]quinolin-4-yl]-2,2,2-trichloroacetamide (15.82 g, 33.83 mmol) as the starting material to yield 8.60 g of 7-bromo-2-(2-methoxyethyl)thiazolo[4,5-c]quinolin-4-amine as light yellow solid. Recrystallization from boiling isopropanol yielded yellow needles of 7-bromo-2-(2-methoxyethyl)thiazolo[4,5-c]quinolin-4-amine, mp 172-175° C. ¹H NMR (300 MHz, d₆-DMSO) δ 7.74 (d, J=2.2 Hz, 1H), 7.72 (d, J=8.4 Hz, 1H), 7.35 (dd, J=8.4 Hz, 1.9, 1H), 7.12 (s, 2H), 3.78 (t, J=5.9 Hz, 2H), 3.39 (t, J=5.9 Hz, 2H), 3.31 (s, 3H); ¹³C NMR (75 MHz, d₆-DMSO) δ 168.6, 152.9, 146.0, 139.2, 137.3, 127.6, 126.5, 124.6, 121.5, 117.7, 70.2, 58.0, 33.9; Anal. calcd for C₁₃H₁₂N₃OS₁Br₁: C, 46.17; H, 3.58; N, 12.42. Found: C, 46.08; H, 3.29; N, 12.16.

The compounds in the tables below were prepared according to the following method. A solution of 7-bromo-2-propylthiazolo[4,5-c]quinolin-4-amine (prepared as described in Parts A-I of Example 1, 32.2 mg, 0.10 mmol) or 7-bromo-2-(2-methoxyethyl)thiazolo[4,5-c]quinolin-4-amine (prepared as described above, 33.8 mg, 0.10 mmol) in 7:3 volume:volume (v:v) dichloromethane:methanol (1 mL) was added to a test tube, and the solvent was removed by vacuum centrifugation. The boronic acid (0.11 mmol) indicated in the table below and n-propanol (1.6 mL) were sequentially added, and the test tube was purged with nitrogen. For Example 24 and 28, the boronic acid used was 5-({[tert-butyl(dimethyl)silyl]oxy}methyl)pyridin-3-ylboronic acid, which was synthesized by modifying procedures published by W. Li et al., J. Org. Chem., 67, pp. 5394-5397 (2002) and N. Zhang et al., J. Med. Chem., 45, pp. 2832-2840 (2002). The reaction mixture was sonicated until it had the consistency of milk. Palladium (II) acetate (0.146 mL of a 0.018 M solution in toluene, 0.0026 mmol), 2M aqueous sodium carbonate solution (602 μL), deionized water (113 μL), and a solution of 0.15 M triphenylphosphine in n-propanol (53 μL, 0.0079 mmol) were sequentially added. The test tube was purged with nitrogen, capped, and then heated to 80° C. overnight in a sand bath. For Examples 24 and 28, the solvent was removed by vacuum centrifugation, and glacial acetic acid (1 mL), tetrahydrofuran (1 mL), and deionized water (1 mL) were added to the test tube. The reaction was heated overnight at 60° C. or 80° C. The solvent was removed from the test tubes by vacuum centrifugation.

The contents of each test tube were passed through a Waters Oasis Sample Extractions Cartridge MCX (6 cc) according to the following procedure. Hydrochloric acid (3 mL of 1 N) was added to adjust each example to pH 5-7, and the resulting solution was passed through the cartridge optionally using light nitrogen pressure. The cartridge was washed with methanol (5 mL) optionally using light nitrogen pressure and transferred to a clean test tube. A solution of 1% ammonia in methanol (2×5 mL) was then passed through the cartridge optionally using light nitrogen pressure, and the basic solution was collected and concentrated.

The compounds were purified by preparative high performance liquid chromatography (prep HPLC) using a Waters Fraction Lynx automated purification system. The prep HPLC fractions were analyzed using a Micromass LC/TOF-MS, and the appropriate fractions were centrifuge evaporated to provide the trifluoroacetate salt of the desired compound. Column: Zorbax BonusRP, 21.2×50 millimeters (mm), 5 micron particle size; non-linear gradient elution from 5-95% B where A is 0.05% trifluoroacetic acid/water and B is 0.05% trifluoroacetic acid/acetonitrile; fraction collection by mass-selective triggering. The table below shows the reagent used for each example, the structure of the resulting compound, and the observed accurate mass for the isolated trifluoroacetate salt.

| | | | |
|---|---|---|---|
| Ex | Reagent | R₃ | Measured Mass (M + H) |

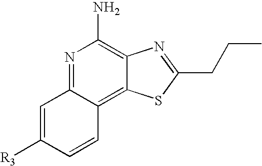

| | | | |
|---|---|---|---|
| 5 | 4-(Hydroxymethyl)phenylboronic acid | 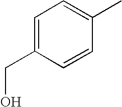 | 350.1362 |
| 6 | (2-Hydroxymethylphenyl)boronic acid dehydrate | 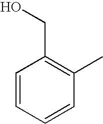 | 350.1337 |
| 7 | 3-(N,N-Di- methylaminocarbonyl)phenylboronic acid | 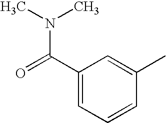 | 391.1570 |
| 8 | 3-(4-Boronophenyl)propionic acid | 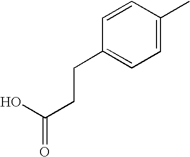 | 392.1436 |
| 9 | 3-(N-Iso- propylaminocarbonyl)phenylboronic acid | 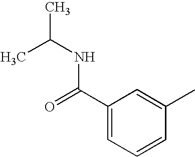 | 405.1737 |
| 10 | 4-Borono-DL-phenylalanine | 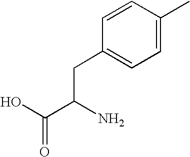 | 407.1543 |
| 11 | 4-(Ethylsulfonyl)phenylboronic acid | 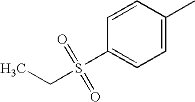 | 412.1118 |
| 12 | 3-(2-Cyano- ethylaminocarbonyl)phenylboronic acid | 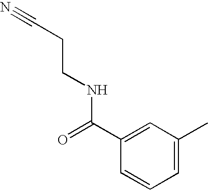 | 416.1528 |
| 13 | 3-(Butylaminocarbonyl)phenylboronic acid | 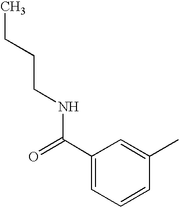 | 419.1900 |
| 14 | 3-(Isobutylaminocarbonyl)phenylboronic acid | 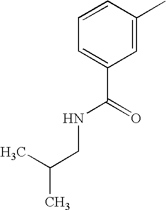 | 419.1927 |
| 15 | 4-(Isobutylaminocarbonyl)phenylboronic acid | 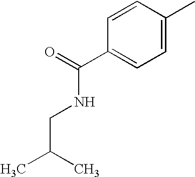 | 419.1907 |
| 16 | 3-(Piperidine-1-carbonyl)phenylboronic acid | 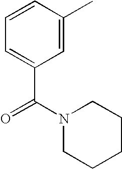 | 431.1881 |
| 17 | 4-(Cyclo- pentylaminocarbonyl)phenylboronic acid | 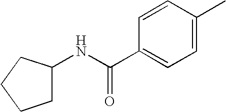 | 431.1898 |
| 18 | 3-(Morpholine-4-carbonyl)phenylboronic acid | 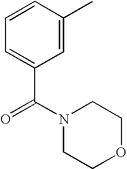 | 433.1711 |
| 19 | 4-(Morpholine-4-carbonyl)phenylboronic acid | 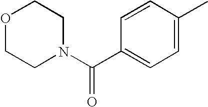 | 433.1704 |
| 20 | 3-(Furfurylaminocarbonyl)phenylboronic acid | 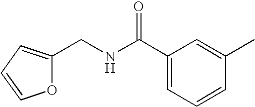 | 443.1528 |
| 21 | 4-Benzyloxy-3-fluorophenylboronic acid | 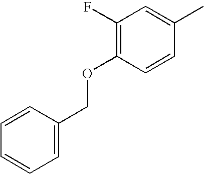 | 444.1540 |
| 22 | 4-(4-Oxopiperidine-1-carbo- nyl)phenylboronic acid | 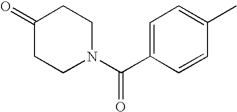 | 445.1679 |
| 23 | 3-(N-Benzylaminocarbonyl)phenylboronic acid | 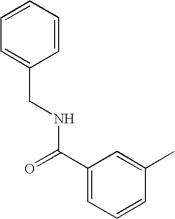 | 453.1748 |
| 24 | 4-({[tert- Butyl(dimethyl)silyl]oxy}methyl)pyridin- 3-ylboronic acid | 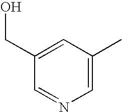 | 351.1284 |
| 25 | (4-Aminomethylphenyl)boronic acid, pinacol ester, HCl | 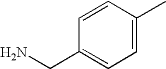 | 349.1463 |
| 26 | 1-(Phenylsulfonyl)-1H-indol-3-ylboronic acid | 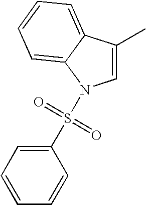 | 499.1215 |

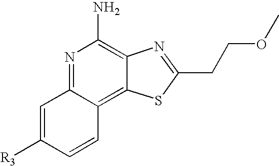

| | | | |
|---|---|---|---|
| 27 | 4-Vinylphenylboronic acid | 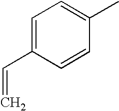 | 362.1311 |
| 28 | 5-({[tert- Butyl(dimethyl)silyl]oxy}methyl)pyridin-3- ylboronic acid | 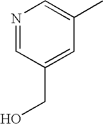 | 367.1245 |
| 29 | (3-Aminocarbonyl)phenylboronic acid | 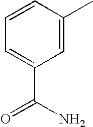 | 379.1231 |
| 30 | 3,4-Methylenedioxyphenylboronic acid | 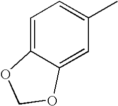 | 380.1064 |
| 31 | [3-(Hydroxypropyl)phenyl]boronic acid | 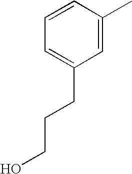 | 394.1596 |
| 32 | 3-(N-Isopropylaminocarbonyl)phenylboronic acid | 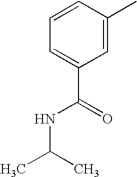 | 421.1711 |
| 33 | 3-(N-Propylaminocarbonyl)phenylboronic acid | 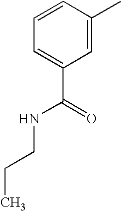 | 421.1684 |
| 34 | 4-Borono-DL-phenylalanine | 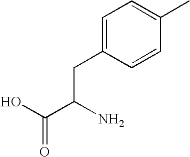 | 423.1499 |
| 35 | 3-(Butylaminocarbonyl)phenylboronic acid | 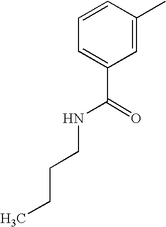 | 435.1833 |
| 36 | 3-(Isobutylaminocarbonyl)phenylboronic acid | 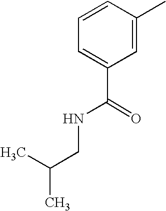 | 435.1849 |
| 37 | 4-(Isobutylaminocarbonyl)phenylboronic acid | 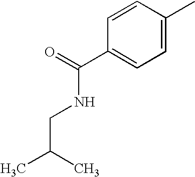 | 435.1833 |
| 38 | 3-(Piperidine-1-carbonyl)phenylboronic acid | 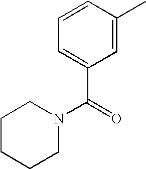 | 447.1839 |
| 39 | 4-(Cyclopentylaminocarbonyl)phenylboronic acid | 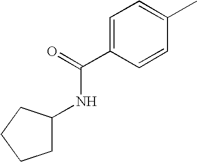 | 447.1892 |
| 40 | 4-Benzyloxy-3-fluorophenylboronic acid | 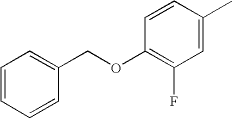 | 460.1492 |
| 41 | 4-(Aminomethylphenyl)boronic acid, pinacol ester, HCl | 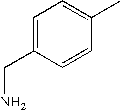 | 365.1440 |

### Example 42

### 3-(4-Amino-2-propylthiazolo[4,5-c]quinolin-7-yl)-N,N-diethylbenzamide

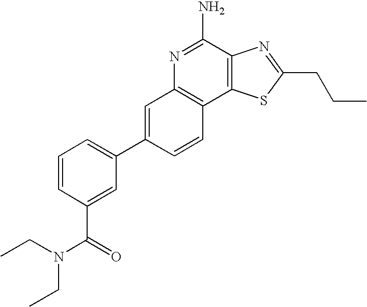

7-Bromo-2-propylthiazolo[4,5-c]quinolin-4-amine (0.50 g, 1.55 mmol) was coupled with 3-(N,N-diethylaminocarbonyl)phenylboronic acid (0.41 g, 1.86 mmol) using the method of Example 3. The crude product was purified by HPFC (silica gel, gradient elution with 0-30% CMA in chloroform) to provide a light yellow solid. The solid was suspended in cold diethyl ether (20 mL), isolated by filtration, rinsed with diethyl ether, and then dried in an oven at 80° C. to provide 0.374 g of 3-(4-amino-2-propylthiazolo[4,5-c]quinolin-7-yl)-N,N-diethylbenzamide as an off-white solid, mp 169-172° C. ¹H NMR (500 MHz, d₆-DMSO) δ 7.70 (d, J=8.2, 1H), 7.85 (d, J=1.9, 1H), 7.83 (d, J=7.9, 1H) 7.67 (s, 1 H), 7.58 (dd, J=8.2, 1.6, 1H), 7.55 (t, J=7.6, 1H), 7.35 (d, J=7.6, 1H), 6.94 (s, 2H), 3.45 (m, 2H), 3.24 (m, 2H) 3.16 (t, J=6.2, 2H), 1.87 (sextet, J=7.5, 2H), 1.16 (m, 3H), 1.09 (m, 3H), 1.02 (t, J=7.5, 3H); ¹³C NMR (125 MHz, d₆-DMSO) δ 170.8, 169.7, 152.5, 145.2, 140.0, 139.7, 138.8, 138.1, 137.7, 129.2, 127.4, 125.4, 125.3, 124.3, 123.5, 121.0, 118.3, 35.2, 22.8, 14.1, 13.5, 12.8; Anal. calcd for C₂₄H₂₆N₄OS: C, 68.87; H, 6.26; N, 13.39. Found: C, 68.72; H, 6.20; N, 13.37.

### Example 43

### 2-Propyl-7-[3-(pyrrolidin-1-ylcarbonyl)phenyl]thiazolo[4,5-c]quinolin-4-amine

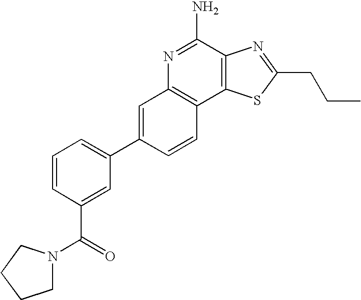

7-Bromo-2-propylthiazolo[4,5-c]quinolin-4-amine (0.50 g, 1.55 mmol) was coupled with 3-(pyrrolidin-1-ylcarbonyl)phenylboronic acid (0.41 g, 1.86 mmol) using the method of Example 3. The crude product was purified by HPFC (silica gel, gradient elution with 0-30% CMA in chloroform) to provide a light yellow solid. The solid was suspended in cold diethyl ether (20 mL), isolated by filtration, rinsed with diethyl ether, and then dried in an oven at 80° C. to provide 0.317 g of 2-propyl-7-[3-(pyrrolidin-1-ylcarbonyl)phenyl]thiazolo[4,5-c]quinolin-4-amine as an off-white solid, mp 177-180° C. ¹H NMR (500 MHz, d₆-DMSO) δ 7.88-7.83 (m, 4H), 7.58 (dd, J=8.2, 1.6, 1H), 7.55 (t, J=7.8, 1H), 7.51 (d, J=7.6, 1H), 6.93 (s, 2H), 3.49 (t, J=6.9, 2H), 3.45 (t, J=6.9, 2H), 3.16 (t, J=6.2, 2H), 1.90-1.81 (m, 6H), 1.02 (t, J=7.5, 3H); ¹³C NMR (125 MHz, d₆-DMSO) δ 170.8, 168.0, 152.5, 145.2, 139.9, 139.7, 138.8, 138.0, 137.6, 129.0, 128.1, 126.2, 125.4, 125.3, 123.5, 121.1, 118.3, 48.9, 45.92, 35.2, 22.8, 13.5; Anal. calcd for C₂₄H₂₄N₄OS: C, 69.20; H, 5.81; N, 13.45. Found: C, 69.01; H, 5.66; N, 13.35.

### Example 44

### N-[4-(4-Amino-2-propylthiazolo[4,5-c]quinolin-7-yl)phenyl]acetamide

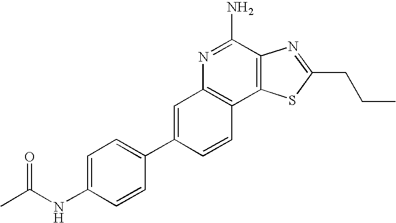

7-Bromo-2-propylthiazolo[4,5-c]quinolin-4-amine (0.50 g, 1.55 mmol) was coupled with 4-(acetylamino)phenylboronic acid (0.33 g, 1.86 mmol) using the method of Example 3. The crude product was purified by HPFC (silica gel, gradient elution with 0-30% CMA in chloroform) to provide a light yellow solid. The solid was suspended in cold diethyl ether (20 mL), isolated by filtration, rinsed with diethyl ether, and then dried in an oven at 80° C. to provide 0.351 g of N-[4-(4-amino-2-propylthiazolo[4,5-c]quinolin-7-yl)phenyl]acetamide as an off-white solid, mp 252-255° C. ¹H NMR (500 MHz, d₆-DMSO) δ 10.0 (s, 1H), 7.83 (d, J=8.2, 1H), 7.80 (d, J=1.9, 1H), 7.71 (s, 4H), 7.54 (dd, J=8.4, 1.9, 1H), 6.89 (s, 2H), 3.15 (t, J=6.2, 2H), 2.07 (s, 3H), 1.87 (sextet, J=7.5, 2H), 1.02 (t, J=7.5, 3H); ¹³C NMR (125 MHz, d₆-DMSO) δ 170.5, 168.3, 152.4, 145.3, 140.0, 139.0, 138.9, 137.4, 134.3, 127.0, 125.2, 122.7, 120.7, 119.3, 117.7, 35.2, 24.0, 22.8, 13.5; Anal. calcd for C₂₁H₂₀N₄OS: C, 67.00; H, 5.36; N, 14.88. Found: C, 66.85; H, 5.19; N, 14.87.

### Example 45

### 3-(4-Amino-2-propylthiazolo[4,5-c]quinolin-7-yl)-N-cyclopropylbenzamide

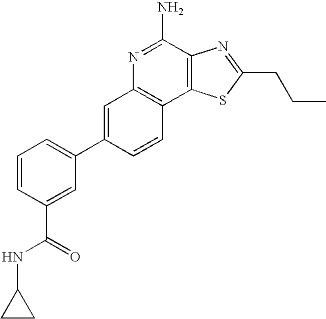

7-Bromo-2-propylthiazolo[4,5-c]quinolin-4-amine (0.50 g, 1.55 mmol) was coupled with 3-(cyclopropylaminocarbonyl)phenylboronic acid (0.38 g, 1.86 mmol) using the method of Example 3. The crude product was purified by HPFC (silica gel, gradient elution with 0-30% CMA in chloroform) to provide a light yellow solid. The solid was suspended in cold diethyl ether (20 mL), isolated by filtration, rinsed with diethyl ether, and then dried in an oven at 80° C. to provide 0.323 g of 3-(4-amino-2-propylthiazolo[4,5-c]quinolin-7-yl)-N-cyclopropylbenzamide as an off-white solid, mp 251-254° C. ¹H NMR (300 MHz, d₆-DMSO) δ 8.62 (d, J=4.1, 1H), 8.20 (s, 1H), 7.95 (d, J=1.9, 1H), 7.92-7.83 (m 3H), 7.62 (dd, J=8.2, 1.6, 1H), 7.56 (t, J=7.5, 1H), 6.92 (s, 2H), 3.16 (t, J=6.2, 2H), 2.88 (m 1H), 1.88 (sextet, J=7.5, 2H), 1.02 (t, J=7.5, 3H), 0.75-0.58 (m, 4H); ¹³C NMR (75 MHz, d₆-DMSO) δ 170.8, 167.2, 152.5, 145.2, 139.9, 139.7, 138.9, 137.6, 135.1, 129.4, 129.0, 126.6, 125.3, 123.6, 121.0, 118.2, 35.2, 23.1, 22.8, 13.5, 5.7; Anal. calcd for C₂₃H₂₂N₄OS: C, 68.63; H, 5.51; N, 13.92. Found: C, 68.50; H, 5.17; N, 13.86.

### Example 46

### N-[2-(4-Amino-2-propylthiazolo[4,5-c]quinolin-7-yl)phenyl]acetamide

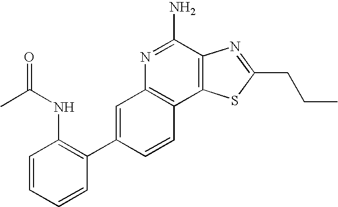

7-Bromo-2-propylthiazolo[4,5-c]quinolin-4-amine (0.50 g, 1.55 mmol) was coupled with 2-(acetylamino)phenylboronic acid (0.33 g, 1.86 mmol) using the method of Example 3. The crude product was purified by HPFC (silica gel, gradient elution with 0-30% CMA in chloroform) to provide a light yellow solid. The solid was suspended in cold diethyl ether (20 mL), isolated by filtration, rinsed with diethyl ether, and then dried in an oven at 80° C. to provide 0.305 g of N-[2-(4-amino-2-propylthiazolo[4,5-c]quinolin-7-yl)phenyl]acetamide as an off-white solid, mp 206-209° C. ¹H NMR (300 MHz, d₆-DMSO) δ 9.30 (s, 1H), 7.82 (d, J=8.1, 1H), 7.58 (d, J=1.6, 1H), 7.51 (d, J=7.8, 1H), 7.41-7.27 (m, 3H) 7.24 (dd, J=8.4, 1.8, 1H), 6.93 (s, 2H), 3.16 (t, J=7.5, 2H), 1.87 (s, 3H), 1.86 (sextet, J=7.5, 2H), 1.02 (t, J=7.5, 3H); ¹³C NMR (75 MHz, d₆-DMSO) δ 170.6, 168.6, 152.4, 144.9, 139.4, 138.9, 137.6, 136.3, 135.0, 130.2, 127.7, 127.1, 125.9, 125.7, 124.5, 123.0, 117.8, 35.2, 23.0, 22.8, 13.4; Anal. calcd for C₂₁H₂₀N₄OS: C, 67.00; H, 5.36; N, 14.88. Found: C, 66.99; H, 5.25; N, 14.84.

### Example 47

### 2-(4-Amino-2-propylthiazolo[4,5-c]quinolin-7-yl)benzamide

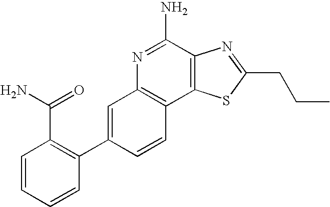

7-Bromo-2-propylthiazolo[4,5-c]quinolin-4-amine (0.50 g, 1.55 mmol) was coupled with 2-(aminocarbonyl)phenylboronic acid (0.31 g, 1.86 mmol) using the method of Example 3. The crude product was purified by HPFC (silica gel, gradient elution with 0-30% CMA in chloroform) to provide a light yellow solid. The solid was suspended in cold diethyl ether (20 mL), isolated by filtration, rinsed with diethyl ether, and then dried in an oven at 80° C. to provide 96 mg of 2-(4-amino-2-propylthiazolo[4,5-c]quinolin-7-yl)benzamide as an off-white solid, mp 274-276° C. ¹H NMR (300 MHz, d₆-DMSO) δ 7.81 (d, J=8.1, 1H), 7.70 (s, 1H), 7.68 (d, J=1.8, 1H), 7.54-7.39 (m, 4H), 7.31 (dd, J=8.4, 1.8, 1H), 7.30 (s, 1H), 6.90 (s, 2H), 3.16 (t, J=7.5, 2H), 1.86 (sextet, J=7.5, 2H), 1.02 (t, J=7.5, 3H); ¹³C NMR (75 MHz, d₆-DMSO) δ 171.1, 170.6, 152.3, 144.7, 140.8, 138.9, 138.7, 137.52, 137.49, 129.9, 129.2, 127.6, 127.1, 125.3, 124.4, 122.8, 117.8, 35.2, 22.7, 13.5; Anal. calcd for C₂₀H₁₈N₄OS: C, 66.28; H, 5.01; N, 15.46. Found: C, 66.15; H, 4.91; N, 15.42.

### Example 48

### 7-[3-(Methylsulfonyl)phenyl]-2-propylthiazolo[4,5-c]quinolin-4-amine

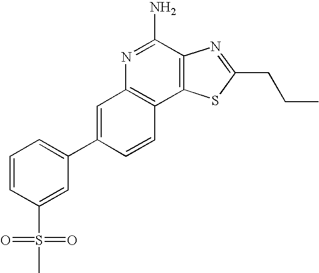

7-Bromo-2-propylthiazolo[4,5-c]quinolin-4-amine (0.50 g, 1.55 mmol) was coupled with 3-methylsulfonylphenylboronic acid (0.37 g, 1.86 mmol) using the method of Example 3. The crude product was purified by HPFC (silica gel, gradient elution with 0-35% CMA in chloroform) to provide a light yellow solid. The solid was suspended in cold diethyl ether (20 mL), isolated by filtration, rinsed with diethyl ether, and then dried in an oven at 80° C. to provide 0.174 g of 7-[3-(methylsulfonyl)phenyl]-2-propylthiazolo[4,5-c]quinolin-4-amine as an off-white solid, mp 242-245° C. ¹H NMR (500 MHz, d₆-DMSO) δ 8.26 (t, J=1.9, 1H), 8.15 (d, J=7.9, 1H), 7.95 (d, J=1.9, 1H), 7.93 (d, J=8.6, 1H), 7.92 (d, J=8.2, 1H), 7.77 (t, J=7.9, 1H), 7.65 (dd, J=8.2, 1.6, 1H), 6.96 (s, 2H), 3.32 (s, 3H), 3.17 (t, J=6.2, 2H), 1.88 (sextet, J=7.5, 2H), 1.03 (t, J=7.5, 3H); ¹³C NMR (125 MHz, d₆-DMSO) δ 171.1, 152.6, 145.2, 141.7, 141.1, 138.8, 138.6, 137.8, 131.9, 130.1, 125.8, 125.6, 125.2, 123.9, 121.0, 118.6, 43.4, 35.2, 22.8, 13.5.

### Example 49

### N-[3-(4-Amino-2-propylthiazolo[4,5-c]quinolin-7-yl)phenyl]acetamide

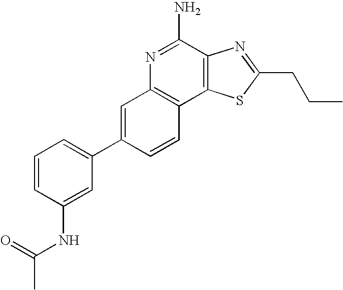

7-Bromo-2-propylthiazolo[4,5-c]quinolin-4-amine (0.50 g, 1.55 mmol) was coupled with 3-(acetylamino)phenylboronic acid (0.33 g, 1.86 mmol) using the method of Example 3. The crude product was purified by HPFC (silica gel, gradient elution with 0-35% CMA in chloroform) to provide a light yellow solid. The solid was suspended in cold diethyl ether (20 mL), isolated by filtration, rinsed with diethyl ether, and then dried in an oven at 80° C. to provide 45 mg of N-[3-(4-amino-2-propylthiazolo[4,5-c]quinolin-7-yl)phenyl]acetamide as an off-white solid, mp 173-176° C. ¹H NMR (500 MHz, d₆-DMSO) δ 10.0 (s, 1H), 8.03, (s, 1H), 7.87 (d, J=8.2, 1H), 7.79 (d, J=1.6, 1H), 7.57 (d, J=7.0, 1H), 7.50 (dd, J=8.2, 1.6, 1H), 7.43-7.39 (m, 2H), 6.93 (s, 2H), 3.16 (t, J=6.2, 2H), 2.07 (s, 3H), 1.87 (sextet, J=7.5, 2H), 1.02 (t, J=7.5, 3H); ¹³C NMR (125 MHz, d₆-DMSO) δ 170.7, 168.4, 152.5, 145.2, 140.4, 140.3, 139.9, 138.9, 137.6, 129.4, 125.3, 123.3, 121.5, 120.9, 118.2, 118.1, 117.3, 35.2, 24.1, 22.8, 13.5.

### Example 50

### 7-(3-Aminomethylphenyl)-2-propylthiazolo[4,5-c]quinolin-4-amine

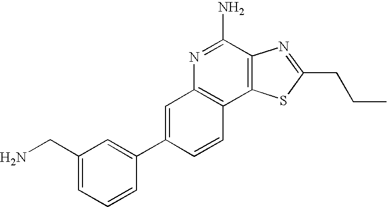

7-Bromo-2-propylthiazolo[4,5-c]quinolin-4-amine (2.15 g, 6.67 mmol) was coupled with 3-(aminomethyl)phenylboronic acid hydrochloride (1.50 g, 8.00 mmol) using the method of Example 3 except that 2.4 equivalents of sodium carbonate was used. The crude product was purified by HPFC (silica gel, gradient elution with 0-40% CMA in chloroform) to provide 1.67 g of a light yellow solid. A portion (0.22 g) of this material was recrystallized from refluxing propyl acetate (15 mL), isolated by filtration, washed with cold propyl acetate, and dried in a vacuum oven at 60° C. to provide 80 mg of 7-(3-aminomethylphenyl)-2-propylthiazolo[4,5-c]quinolin-4-amine as a light yellow solid, mp 168-171° C. ¹H NMR (500 MHz, d₆-DMSO) δ 7.85 (m, 3H), 7.74 (s, 1H), 7.59-7.56 (m, 3H), 7.41 (t, J=7.6, 1H), 7.33 (d, J=7.5, 1H), 6.90 (s, 2H), 3.79 (s, 2H), 3.16 (t, J=7.6, 2H), 7.88 (sextet, J=7.5, 2H), 1.02 (t, J=7.2, 3H); ¹³C NMR (125 Hz, d₆-DMSO) δ 170.6, 152.4, 145.2, 145.1, 140.7, 139.8, 138.9, 137.5, 128.7, 126.4, 125.6, 125.2, 124.6, 123.3, 121.1, 118.0, 45.7, 35.2, 22.8, 13.5; Anal, calcd for C₂₀H₂₀N₄S: C, 68.94; H, 5.79; N, 16.08. Found: C, 68.96; H, 5.71; N, 16.03.

### Example 51

### 7-[2-(Aminomethyl)-4-fluorophenyl)-2-propylthiazolo[4,5-c]quinolin-4-amine

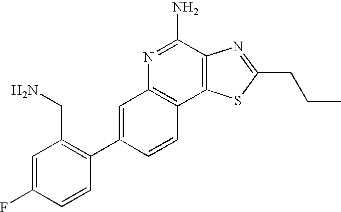

7-Bromo-2-propylthiazolo[4,5-c]quinolin-4-amine (0.50 g, 1.55 mmol) was coupled with 2-(aminomethyl)-4-fluorophenylboronic acid hydrochloride (0.38 g, 1.86 mmol) using the method of Example 3 except that 2.4 equivalents of sodium carbonate was used. The crude product was purified by HPFC (silica gel, gradient elution with 0-35% CMA in chloroform) to provide a light yellow solid. The solid was suspended in cold diethyl ether (20 mL), isolated by filtration, rinsed with diethyl ether, and then dried in an oven at 80° C. to provide 0.155 g of 7-[2-(aminomethyl)-4-fluorophenyl)-2-propylthiazolo[4,5-c]quinolin-4-amine as an off-white solid, mp 181-184° C. ¹H NMR (500 MHz, d₆-DMSO) δ 7.82 (d, J=8.1, 1H), 7.51 (d, J=1.6, 1H), 7.47 (dd, J=10.6, 2.5, 1H), 7.30 (dd, J=8.4, 6.0, 1H), 7.21 (dd, J=8.1, 1.6, 1H), 7.11 (td, J=8.5, 2.5, 1H), 6.93 (s, 2H), 3.67 (s, 2H), 3.16 (t, J=6.2, 2H), 1.87 (sextet, J=7.5, 2H), 1.84 (s, 2H), 1.02 (t, J=7.5, 3H); ¹³C NMR (125 MHz, d₆-DMSO) δ 170.7, 163.4, 152.5, 144.7, 144.3, 140.2, 138.9, 137.6, 136.4, 131.2, 125.9, 124.5, 123.5, 117.7, 114.3, 112.8, 43.1, 35.2, 22.8, 13.4.

### Examples 52-65

7-Bromo-2-ethoxymethylthiazolo[4,5-c]quinolin-4-amine, which was used as a substrate in the examples 52-65, was prepared from 3-amino-7-bromoquinolin-4-ol hydrochloride as described below.

Part A

Under a nitrogen atmosphere, a mixture of 3-amino-7-bromoquinolin-4-ol hydrochloride (19.0 g, 69 mmol), anhydrous dichloromethane (275 mL), and triethylamine (19.2 mL, 138 mmol) was cooled for 10 minutes in a 0° C. ice bath. Ethoxyacetyl chloride (8.87 g, 72.4 mmol) was added dropwise. The solution was allowed to stir for 10 minutes, the ice bath was removed, and the reaction mixture was allowed to stir for about 5 hours. A solid was isolated by filtration, washed with a small amount of dichloromethane, and then dried overnight in a vacuum oven at 60° C. to provide 25.63 g of N-(7-bromo-4-hydroxyquinolin-3-yl)-2-ethoxyacetamide as a light brown solid.

Part B

Using the general method of Part F of Example 1, N-(7-bromo-4-hydroxyquinolin-3-yl)-2-ethoxyacetamide (22.29 g, 68.97 mmol) was reacted with phosphorous pentasulfide (15.33 g, 34.49 mmol). The reaction was worked up and the crude product was purified using the methods described in Part F of Example 1 to provide 6.0 g of 7-bromo-2-ethoxymethylthiazolo[4,5-c]quinoline as a light yellow solid.

Part C

Using the general method of Part G of Example 1, 7-bromo-2-ethoxymethylthiazolo[4,5-c]quinoline (5.97 g) was oxidized to provide 6.27 g of 7-bromo-2-ethoxymethylthiazolo[4,5-c]quinoline 5-oxide as a light yellow powder.

Part D

Using the general methods of Part H and Part I, 7-bromo-2-ethoxymethylthiazolo[4,5-c]quinoline 5-oxide (6.27 g) was converted to 7-bromo-2-ethoxymethylthiazolo[4,5-c]quinolin-4-amine (5.10 g of a light yellow solid). A portion (0.31 g) was recrystallized from refluxing isopropanol (15 mL), isolated by filtration, rinsed with cold isopropanol, and dried in a vacuum oven at 60° C. to provide 0.13 g of pure 7-bromo-2-ethoxymethylthiazolo[4,5-c]quinolin-4-amine, mp 201-204° C. Anal. calcd for C₁₃H₁₂N₃OS: C, 46.17; H, 3.58; N, 12.42. Found: C, 46.12; H, 3.51; N, 12.19.

The compounds in the table below were prepared and purified using the methods described in Examples 5-41. 7-Bromo-2-ethoxymethylthiazolo[4,5-c]quinolin-4-amine was used in lieu of 7-bromo-2-propylthiazolo[4,5-c]quinolin-4-amine. The table below shows the reagent used for each example, the structure of the resulting compound, and the observed accurate mass for the isolated trifluoroacetate salt.

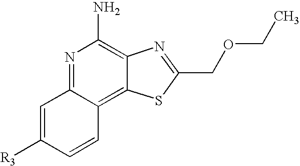

| Ex | Reagent | R₃ | Measured Mass (M + H) |
|---|---|---|---|
| 52 | 4-Vinylphenylboronic acid | 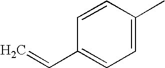 | 362.1304 |
| 53 | 3,4-Methylenedioxyphenylboronic acid | 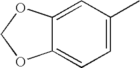 | 380.1044 |
| 54 | [3-(Hydroxypropyl)phenyl]boronic acid | 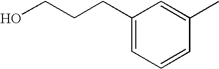 | 394.1602 |
| 55 | (3-Aminomethylphenyl)boronic acid, hydrochloride | 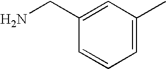 | 365.1472 |
| 56 | 4-(Cyclopropylaminocarbonyl)phenylboronic acid | 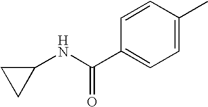 | 419.1517 |
| 57 | 3-(N-Isopropylaminocarbonyl)phenylboronic acid | 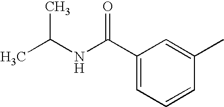 | 421.1707 |
| 58 | 3-(N-Propylaminocarbonyl)phenylboronic acid | 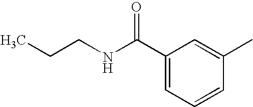 | 421.1685 |
| 59 | 3-(Isobutylaminocarbonyl)phenylboronic acid | 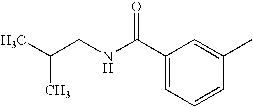 | 435.1870 |
| 60 | 4-(Isobutylaminocarbonyl)phenylboronic acid | 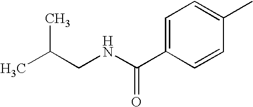 | 435.1834 |
| 61 | 3-(Piperidine-1-carbonyl)phenylboronic acid | 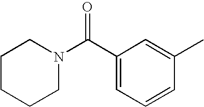 | 447.1850 |
| 62 | 3-(Furfurylaminocarbonyl)phenylboronic acid | 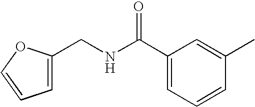 | 459.1508 |
| 63 | 3-(N-Benzylaminocarbonyl)phenylboronic acid | 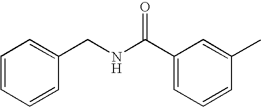 | 469.1738 |
| 64 | (4-Aminomethylphenyl)boronic acid, pinacol ester, hydrochloride | 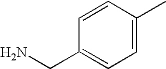 | 365.1426 |
| 65 | 1-(Phenylsulfonyl)-1H-indol-3-ylboronic acid | 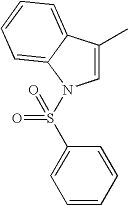 | 515.1216 |

### Examples 66-90

Part A

7-bromo-2-(2-methoxyethyl)thiazolo[4,5-c]quinolin-4-amine (2.75 g, 8.13 mmol) was coupled with 4-(aminomethyl)phenylboronic acid hydrochloride (1.83 g, 9.8 mmol) using the method of Example 3. The crude product was purified by HPFC (silica gel, gradient elution with 0-30% CMA in chloroform) to provide 1.03 g of 7-(4-aminomethyl)phenyl-2-(2-methoxyethyl)thiazolo[4,5-c]quinolin-4-amine a light yellow solid.

Part B

A reagent from the table below (1.1 equivalents) was added to a test tube containing a solution of 7-(4-aminomethyl)phenyl-2-(2-methoxyethyl)thiazolo[4,5-c]quinolin-4-amine (36.9 mg, 1.0 equivalents) in N,N-dimethylacetamide (1 mL) containing N,N-diisopropylethylamine (2.0 eq). The test tube was capped and placed on a shaker at ambient temperature overnight (approximately 18 hours). The reaction was quenched with water (2 drops). The solvent was removed by vacuum centrifugation. The compounds were purified by preparative high performance liquid chromatography (prep HPLC) using a Waters FractionLynx automated purification system. The prep HPLC fractions were analyzed using a Waters LC/TOF-MS, and the appropriate fractions were centrifuge evaporated to provide the trifluoroacetate salt of the desired compound. Reversed phase preparative liquid chromatography was performed with non-linear gradient elution from 5-95% B where A is 0.05% trifluoroacetic acid/water and B is 0.05% trifluoroacetic acid/acetonitrile. Fractions were collected by mass-selective triggering. The table below shows the reagent used for each example, the structure of the resulting compound, and the observed accurate mass for the isolated trifluoroacetate salt.

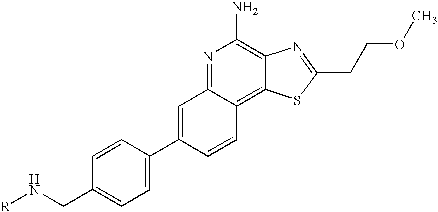

| | | | Measured |
|---|---|---|---|
| Ex- | | | Mass |
| ample | Reagent | R | (M + H) |
| 66 | Acetyl chloride | 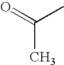 | 407.1548 |
| 67 | Propionyl chloride | 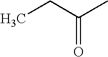 | 421.1713 |
| 68 | Cyclo- propanecarbonyl chloride | 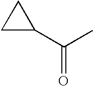 | 433.1705 |
| 69 | Butyryl chloride | 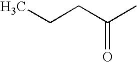 | 435.1883 |
| 70 | Isobutyryl chloride | 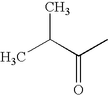 | 435.1831 |
| 71 | Cyclo- butanecarbonyl chloride | 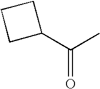 | 447.1891 |
| 72 | Cyclo- hexanecarbonyl chloride | 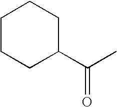 | 475.2144 |
| 73 | Isonicotinoyl chloride hydrochloride | 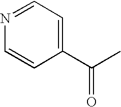 | 470.1693 |
| 74 | Nicotinoyl chloride hydrochloride | 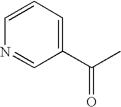 | 470.1691 |
| 75 | Methanesulfonyl chloride | 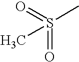 | 443.1222 |
| 76 | Ethanesulfonyl chloride | 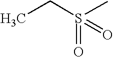 | 457.1357 |
| 77 | 1-Propanesulfonyl chloride | 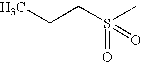 | 471.1510 |
| 78 | 1-Butanesulfonyl chloride | 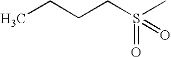 | 485.1676 |
| 79 | Methyl isocyanate | 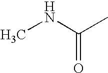 | 422.1677 |
| 80 | Ethyl isocyanate | 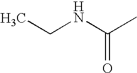 | 436.1837 |
| 81 | Isopropyl isocyanate | 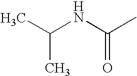 | 450.1985 |
| 82 | n-Butyl isocyanate | 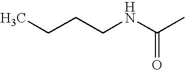 | 464.2162 |
| 83 | Cyclopropyl isothiocyanate | 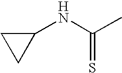 | 464.1618 |
| 84 | Cyclopentyl isocyanate | 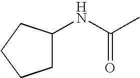 | 476.2092 |
| 85 | Cyclohexyl isocyanate | 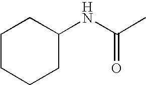 | 490.2291 |
| 86 | N,N- Dimethylcarbamoyl chloride | 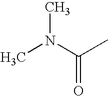 | 436.1830 |
| 87 | 1-Piperidine- carbonyl chloride | 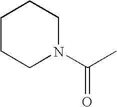 | 476.2140 |
| 88 | 2-Oxo-1- Imidazolidine- carbonyl chloride | 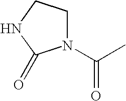 | 477.1733 |
| 89 | 4- Morpholinyl- carbonyl chloride | 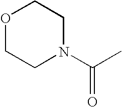 | 478.1954 |
| 90 | 4-Meth- yl-1-piperazine- carbonyl chloride | 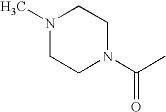 | 491.2264 |

### Examples 91-114

The compounds in the table below were prepared using the method of Part B of Examples 66-90 using 7-(3-aminomethyl)phenyl-2-propylthiazolo[4,5-c]quinolin-4-amine in lieu of 7-(4-aminomethyl)phenyl-2-(2-methoxyethyl)thiazolo[4,5-c]quinolin-4-amine. The table below shows the reagent used for each example, the structure of the resulting compound, and the observed accurate mass for the isolated trifluoroacetate salt.

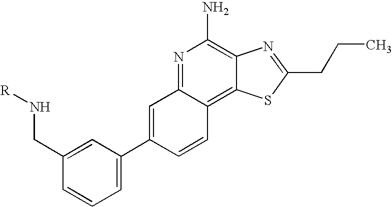

| Ex- | | | Measured Mass |
|---|---|---|---|
| ample | Reagent | R | (M + H) |
| 91 | Propionyl chloride | 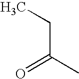 | 405.1767 |
| 92 | Methyl chloroformate | 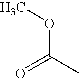 | 407.1520 |
| 93 | Cyclopropanecarbonyl chloride | 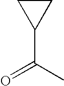 | 417.1712 |
| 94 | Butyryl chloride | 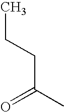 | 419.1897 |
| 95 | Isobutyryl chloride | 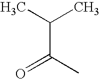 | 419.1915 |
| 96 | Cyclobutanecarbonyl chloride | 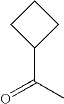 | 431.1918 |
| 97 | Cyclopentanecarbonyl chloride | 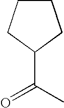 | 445.2046 |
| 98 | Benzoyl chloride | 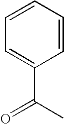 | 453.1737 |
| 99 | Methanesulfonyl chloride | 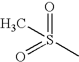 | 427.1282 |
| 100 | Ethanesulfonyl chloride | 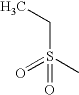 | 441.1412 |
| 101 | 1-Propanesulfonyl chloride | 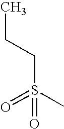 | 455.1601 |
| 102 | Isopropylsulfonyl chloride | 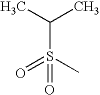 | 455.1581 |
| 103 | Dimethylsulfamoyl chloride | 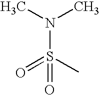 | 456.1487 |
| 104 | 1-Butanesulfonyl chloride | 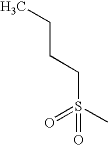 | 469.1739 |
| 105 | 1-Methylimidazole-4- sulfonyl chloride | 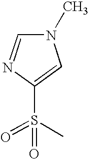 | 493.1484 |
| 106 | Ethyl isocyanate | 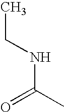 | 420.1855 |
| 107 | Isopropyl isocyanate | 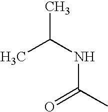 | 434.2010 |
| 108 | n-Propyl isocyanate | 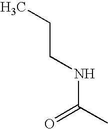 | 434.2017 |
| 109 | Cyclopropyl isothiocyanate | 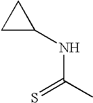 | 448.1609 |
| 110 | Cyclopentyl isocyanate | 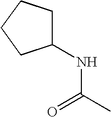 | 460.2130 |
| 111 | Phenyl isocyanate | 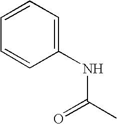 | 468.1835 |
| 112 | 2-Phenyl ethylisocyanate | 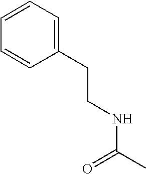 | 496.2164 |
| 113 | 1-Pyrrolidinecarbonyl chloride | 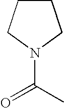 | 446.2010 |
| 114 | 1-Piperidinecarbonyl chloride | 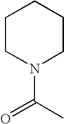 | 460.2170 |

### Examples 115-133

Part A

7-Bromo-2-ethoxymethylthiazolo[4,5-c]quinolin-4-amine (2.25 g, 6.67 mmol) was coupled with 3-(aminomethyl)phenylboronic acid hydrochloride (1.50 g, 8.00 mmol) using the method of Example 3. The crude product was purified by HPFC (silica gel, gradient elution with 0-40% CMA in chloroform) to provide 1.12 g of 7-(3-aminomethyl)phenyl-2-ethoxymethylthiazolo[4,5-c]quinolin-4-amine a light yellow solid.

Part B

The compounds in the table below were prepared using the method of Part B of Examples 66-90 using 7-(3-aminomethyl)phenyl-2-ethoxymethylthiazolo[4,5-c]quinolin-4-amine in lieu of 7-(4-aminomethyl)phenyl-2-(2-methoxyethyl)thiazolo[4,5-c]quinolin-4-amine. The table below shows the reagent used for each example, the structure of the resulting compound, and the observed accurate mass for the isolated trifluoroacetate salt.

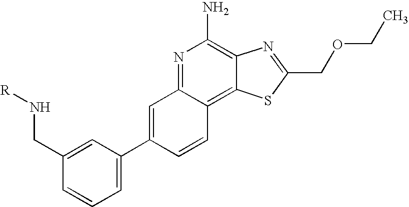

| Ex- | | | Measured Mass |
|---|---|---|---|
| ample | Reagent | R | (M + H) |
| 115 | Acetyl chloride | 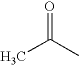 | 407.1571 |
| 116 | Propionyl chloride | 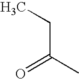 | 421.1739 |
| 117 | Cyclopropanecarbonyl chloride | 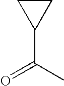 | 433.1717 |
| 118 | Isobutyryl chloride | 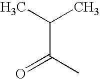 | 435.1892 |
| 119 | Cyclopentanecarbonyl chloride | 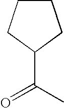 | 461.2035 |
| 120 | Benzoyl chloride | 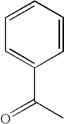 | 469.1703 |
| 121 | Nicotinoyl chloride hydrochloride | 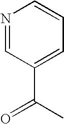 | 470.1680 |
| 122 | Methanesulfonyl chloride | 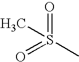 | 443.1197 |
| 123 | Ethanesulfonyl chloride | 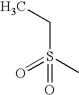 | 457.1377 |
| 124 | 1-Propanesulfonyl chloride | 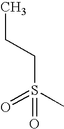 | 471.1495 |
| 125 | Isopropylsulfonyl chloride | 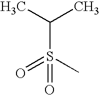 | 471.1536 |
| 126 | Benzenesulfonyl chloride | 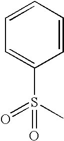 | 505.1389 |
| 127 | 2,2,2- Trifluoroethanesulfonyl chloride | 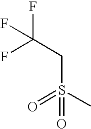 | 511.1107 |
| 128 | Methyl isocyanate | 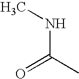 | 422.1651 |
| 129 | Ethyl isocyanate | 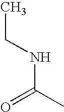 | 436.1841 |
| 130 | N-Propyl isocyanate | 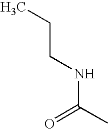 | 450.1989 |
| 131 | Phenyl isocyanate | 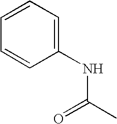 | 484.1795 |
| 132 | 1-Pyrrolidinecarbonyl chloride | 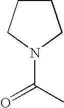 | 462.1973 |
| 133 | 1-Piperidinecarbonyl chloride | 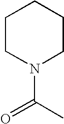 | 476.2123 |

### Example 134

### 8-[3-(Methylsulfonyl)phenyl]-2-propylthiazolo[4,5-c]quinolin-4-amine

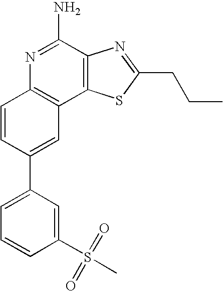 Part A

8-Bromo-2-propylthiazolo[4,5-c]quinolin-4-amine was prepared according to the general method of Example 1 Parts A through I using 4-bromoaniline in lieu of 3-bromoaniline in Part A.

Part B

8-Bromo-2-propylthiazolo[4,5-c]quinolin-4-amine (0.50 g, 1.55 mmol) was coupled with 3-methylsulfonylphenylboronic acid (0.37 g, 1.86 mmol) using the method of Example 3. The crude product was purified by HPFC (silica gel, gradient elution with 0-30% CMA in chloroform) to provide an off-white solid. The solid was suspended in cold diethyl ether (20 mL), isolated by filtration, rinsed with diethyl ether, and then dried in an oven at 80° C. to provide 0.503 g of 8-[3-(methylsulfonyl)phenyl]-2-propylthiazolo[4,5-c]quinolin-4-amine as an off-white solid, mp 252-255° C. ¹H NMR (300 MHz, d₆-DMSO) δ 8.29 (t, J=1.9, 1H), 8.18-8.15 (m, 2H), 7.94-7.88 (m, 2H), 7.77 (d, J=7.8, 1H), 7.72 (d, J=8.7, 1H), 7.02 (s, 2H), 3.32 (s, 3H), 3.17 (t, J=7.2, 2H), 1.88 (sextet, J=7.5, 2H), 1.03 (t, J=7.2, 3H); ¹³C NMR (75 MHz, d₆-DMSO) δ 171.1, 152.6, 144.9, 141.7, 140.9, 139.3, 137.9, 131.8, 131.7, 130.0, 127.5, 126.7, 125.2, 124.8, 122.9, 119.2, 79.1, 35.2, 22.7, 13.4; Anal. calcd for C₂₀H₁₉N₃O₂S₂: C, 60.43; H, 4.82; N, 10.57. Found: C, 60.30; H, 4.57; N, 10.60.

### Example 135

### N-[3-(4-Amino-2-propylthiazolo[4,5-c]quinolin-8-yl)phenyl]acetamide

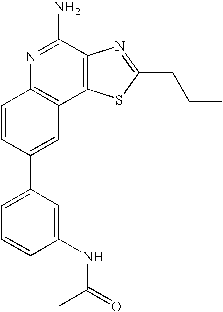

8-Bromo-2-propylthiazolo[4,5-c]quinolin-4-amine (0.50 g, 1.55 mmol) was coupled with 3-(acetylamino)phenylboronic acid (0.33 g, 1.86 mmol) using the method of Example 3. The crude product was purified by HPFC (silica gel, gradient elution with 0-30% CMA in chloroform) to provide a light yellow solid. The solid was suspended in cold diethyl ether (20 mL), isolated by filtration, rinsed with diethyl ether, and then dried in an oven at 80° C. to provide 0.229 g of N-[3-(4-amino-2-propylthiazolo[4,5-c]quinolin-8-yl)phenyl]acetamide as an off-white solid, mp 242-245° C. ¹H NMR (300 MHz, d₆-DMSO) δ 10.0 (s, 1H), 7.94 (t, J=1.9, 1H), 7.93 (d, J=2.2, 1H), 7.76 (dd, J=8.8, 2.2, 1H), 7.68 (d, J=8.7, 1H), 7.60 (dt, J=7.5, 1.6, 1H), 7.44 (dt, J=7.8, 1.9, 1H), 7.40 (t, J=7.8, 1H), 6.96 (s, 2H), 3.16 (t, J=7.5, 2H), 2.07 (s, 3H), 1.87 (sextet, J=7.4, 2H), 1.02 (t, J=7.2, 3H); ¹³C NMR (75 MHz, d₆-DMSO) δ 170.8, 168.4, 152.3, 144.4, 140.1, 139.9, 139.2, 137.8, 129.3, 127.4, 126.5, 122.0, 121.4, 119.1, 117.8, 117.1, 35.2, 24.0, 22.7, 13.4; Anal. calcd for C₂₁H₂₀N₄OS: C, 67.00; H, 5.36; N, 14.88. Found: C, 66.63; H, 4.98; N, 14.43.

### Example 136

### 8-[2-(Aminomethyl)-4-fluorophenyl]-2-propylthiazolo[4,5-c]quinolin-4-amine

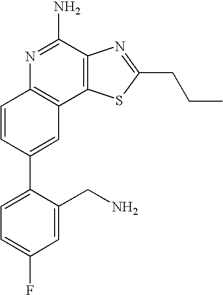

8-Bromo-2-propylthiazolo[4,5-c]quinolin-4-amine (0.50 g, 1.55 mmol) was coupled with 2-(aminomethyl)-4-fluorophenylboronic acid hydrochloride (0.38 g, 1.86 mmol) using the method of Example 3 except that 2.4 equivalents of sodium carbonate was used. The crude product was purified by HPFC (silica gel, gradient elution with 0-30% CMA in chloroform) to provide a light yellow solid. The solid was suspended in cold diethyl ether (20 mL), isolated by filtration, rinsed with diethyl ether, and then dried in an oven at 80° C. to provide 0.215 g of 8-[2-(aminomethyl)-4-fluorophenyl]-2-propylthiazolo[4,5-c]quinolin-4-amine as an off-white solid, mp 198-201° C. ¹H NMR (300 MHz, d₆-DMSO) δ 7.72 (d, J=1.9, 1H), 7.63 (d, J=8.5, 1H), 7.48 (dd, J=8.5, 1.9, 1H), 7.46 (dd, J=10.9, 2.8, 1H), 7.31 (dd, J=8.4, 5.9, 1H), 7.10 (td, J=8.4, 2.9, 1H), 6.93 (s, 2H), 3.66 (s, 2H), 3.14 (t, J=7.5, 2H), 1.85 (sextet, J=7.2, 2H), 1.84 (s, 2H), 1.01 (t, J=7.5, 3H); ¹³C NMR (75 MHz, d₆-DMSO) δ 170.8, 163.4, 152.3, 144.4, 143.9, 139.1, 137.8, 136.1, 133.5, 131.5, 130.0, 125.6, 124.7, 118.6, 114.5, 112.7, 43.1, 35.1, 22.8, 13.4.

### Example 137

### N-[4-(4-Amino-2-propylthiazolo[4,5-c]quinolin-8-yl)phenyl]acetamide

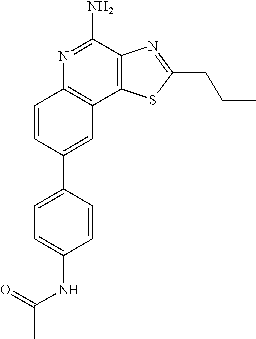

8-Bromo-2-propylthiazolo[4,5-c]quinolin-4-amine (0.50 g, 1.55 mmol) was coupled with 4-(acetylamino)phenylboronic acid (0.33 g, 1.86 mmol) using the method of Example 3. The crude product was purified by HPFC (silica gel, gradient elution with 0-30% CMA in chloroform) to provide a light yellow solid. The solid was suspended in cold diethyl ether (20 mL), isolated by filtration, rinsed with diethyl ether, and then dried in an oven at 80° C. to provide 48 mg of N-[4-(4-amino-2-propylthiazolo[4,5-c]quinolin-8-yl)phenyl]acetamide as an off-white solid, mp 251-254° C. ¹H NMR (300 MHz, d₆-DMSO) δ 10.0 (s, 1H), 7.98 (d, J=2.2, 1H), 7.81 (dd, J=8.7, 2.2, 1H), 7.75-7.63 (m, 5H), 6.90 (s, 2H), 3.17 (t, J=7.5, 2H), 2.06 (s, 3H), 1.88 (sextet, J=7.5, 2H), 1.03 (t, J=7.2, 3H); ¹³C NMR (75 MHz, d₆-DMSO) δ 170.8, 168.2, 152.1, 144.0, 138.6, 134.1, 133.4, 127.2, 126.8, 126.4, 121.5, 119.3, 119.2, 35.2, 24.0, 22.7, 13.4.

### Example 138

### N-[3-(4-Amino-2-propylthiazolo[4,5-c]quinolin-8-yl)phenyl]methanesulfonamide

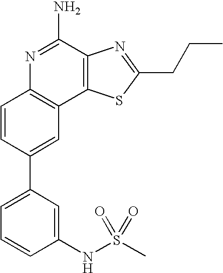

8-Bromo-2-propylthiazolo[4,5-c]quinolin-4-amine (0.500 g, 1.55 mmol) was coupled with 3-(methanesulfonylamino)phenylboronic acid (0.400 g, 1.86 mmol) using the method of Example 3. The crude product was recrystallized from acetonitrile and dried to provide 0.150 g of N-[3-(4-amino-2-propylthiazolo[4,5-c]quinolin-8-yl)phenyl]methanesulfonamide as tan crystals, mp 244-246° C.; 1H NMR (300 MHz, DMSO-d₆) δ 9.82 (s, 1 H), 7.96 (d, J=1.8 Hz, 1 H), 7.90 (dd, J=8.7, 2.0 Hz, 1 H), 7.71 (d, J=8.7 Hz, 1 H), 7.58 (s, 1 H), 7.54 (d, J=7.9 Hz, 1 H), 7.46 (t, J=7.7 Hz, 1 H), 7.24 (d, J=7.8 Hz, 1 H), 6.99 (s, 2 H), 3.17 (t, J=7.5 Hz, 2 H), 3.06 (s, 3 H), 1.95-1.83 (m, 2 H), 1.04 (t, J=7.3 Hz, 3 H); ¹³C NMR (75 MHz, DMSO-d₆) δ 171.3, 152.7, 144.9, 141.2, 139.6, 139.4, 138.2, 133.7, 130.3, 127.8, 127.0, 122.7, 122.6, 119.5, 118.8, 118.4, 35.6, 23.1, 13.8; MS (ESI) m/z 413.02 (M+H)⁺; Anal. Calcd for C₂₀H₂₀N₄O₂S₂: C, 58.23; H, 4.89; N, 13.58; Found: C, 58.19; H, 4.61; N, 13.65.

### Example 139

### [2-(4-Amino-2-propylthiazolo[4,5-c]quinolin-8-yl)phenyl]methanol

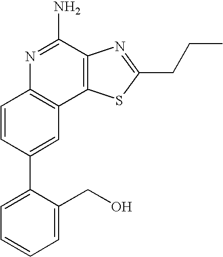

8-Bromo-2-propylthiazolo[4,5-c]quinolin-4-amine (0.500 g, 1.55 mmol) was coupled with 2-(hydroxymethyl)phenylboronic acid (0.249 g, 1.86 mmol) using the method of Example 3. The crude product was purified by HPFC (silica gel, gradient elution with 0-35% CMA in chloroform) to provide an off-white solid. The solid was triturated with diethyl ether (10 mL), isolated by filtration, and then dried under vacuum at 100° C. to provide 0.294 g of [2-(4-amino-2-propylthiazolo[4,5-c]quinolin-8-yl)phenyl]methanol as an off-white solid, mp 194-196° C.; ¹H NMR (300 MHz, DMSO-d₆) δ 7.79 (d, J=1.8 Hz, 1 H), 7.68 (d, J=8.6 Hz, 1 H), 7.62-7.60 (m, 1 H), 7.56 (dd, J=8.5, 2.0 Hz, 1 H), 7.44-7.38 (m, 1 H), 7.37-7.35 (m, 2 H), 6.96 (s, 2 H), 5.19 (t, J=5.3 Hz, 1 H), 4.47 (d, J=5.3 Hz, 2 H), 3.15 (t, J=7.4 Hz, 2 H), 1.93-1.81 (m, 2 H), 1.02 (t, J=7.3 Hz, 3 H); ¹³C NMR (75 MHz, DMSO-d₆) δ 171.2, 152.7, 144.3, 140.2, 139.7, 139.6, 138.1, 134.5, 130.3, 130.0, 128.8, 127.6, 127.3, 126.0, 125.0, 119.0, 61.4, 35.5, 23.1, 13.8; MS (ESI) m/z 350.11 (M+H)⁺; Anal. Calcd for C₂₀H₁₉N₃OS: C, 68.74; H, 5.48; N, 12.02; Found: C, 68.48; H, 5.34; N, 12.04.

### Example 140

### [3-(4-Amino-2-propylthiazolo[4,5-c]quinolin-8-yl)phenyl]methanol

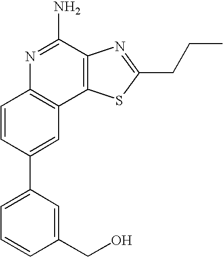

8-Bromo-2-propylthiazolo[4,5-c]quinolin-4-amine (0.500 g, 1.55 mmol) was coupled with 3-(hydroxymethyl)phenylboronic acid (0.249 g, 1.86 mmol) using the method of Example 3. The crude product was purified by HPFC (silica gel, gradient elution with 0-35% CMA in chloroform) to provide an off-white solid. The solid was triturated with diethyl ether (10 mL), isolated by filtration, and then dried under vacuum at 100° C. to provide 0.354 g of [3-(4-amino-2-propylthiazolo[4,5-c]quinolin-8-yl)phenyl]methanol as an off-white solid, mp 176-177° C.; ¹H NMR (300 MHz, DMSO-d₆) δ 8.01 (d, J=1.9 Hz, 1 H), 7.84 (dd, J=8.7, 2.1 Hz, 1 H), 7.74 (s, 1 H), 7.69 (d, J=8.6 Hz, 1 H), 7.66 (d, J=7.8 Hz, 1 H), 7.45 (t, J=7.6 Hz, 1 H), 7.32 (d, J=7.6 Hz, 1 H), 6.95 (s, 2 H), 5.25 (t, J=5.8 Hz, 1 H), 4.61 (d, J=5.8 Hz, 2 H), 3.17 (t, J=7.4 Hz, 2 H), 1.95-1.83 (m, 2 H), 1.04 (t, J=7.4 Hz, 3 H); ¹³C NMR (75 MHz, DMSO-d₆) δ 171.2, 152.6, 144.7, 143.6, 139.8, 139.6, 138.2, 134.3, 129.1, 127.9, 126.9, 125.7, 125.4, 125.1, 122.4, 119.5, 63.3, 35.6, 23.1, 13.8; MS (ESI) m/z 350.11 (M+H)⁺; Anal. Calcd for C₂₀H₁₉N₃OS: C, 68.74; H, 5.48; N, 12.02; Found: C, 68.47; H, 5.19; N, 12.02.

### Example 141

### [4-(4-Amino-2-propylthiazolo[4,5-c]quinolin-8-yl)phenyl]methanol

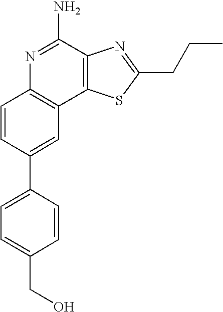

8-Bromo-2-propylthiazolo[4,5-c]quinolin-4-amine (0.500 g, 1.55 mmol) was coupled with 4-(hydroxymethyl)phenylboronic acid (0.249 g, 1.86 mmol) using the method of Example 3. The crude product was purified by HPFC (silica gel, gradient elution with 0-35% CMA in chloroform) to provide an off-white solid. The solid was triturated with diethyl ether (10 mL), isolated by filtration, and then dried under vacuum at 100° C. to provide 0.354 g of [4-(4-amino-2-propylthiazolo[4,5-c]quinolin-8-yl)phenyl]methanol as a yellow solid, mp 212-214° C.; ¹H NMR (300 MHz, DMSO-d₆) δ 8.01 (d, J=1.9 Hz, 1 H), 7.84 (dd, J=8.7, 2.1 Hz, 1 H), 7.76 (d, J=8.2 Hz, 2 H), 7.68 (d, J=8.3 Hz, 1 H), 7.43 (d, J=8.2 Hz, 2 H), 6.94 (s, 2 H), 5.22 (t, J=5.7 Hz, 1 H), 4.56 (d, J=5.7 Hz, 2 H), 3.17 (t, J=7.4Hz, 2 H), 1.95-1.83 (m, 2 H), 1.04 (t, J=7.3 Hz, 3 H); ¹³C NMR (75 MHz, DMSO-d₆) δ 171.2, 152.6, 144.6, 141.9, 139.6, 138.4, 138.2, 134.1, 127.8, 127.4, 126.8, 126.7, 122.3, 119.6, 63.0, 35.6, 23.1, 13.8; MS (ESI) m/z 350.13 (M+H)⁺.

### Example 142

### 8-[3-(Aminomethyl)phenyl]-2-propylthiazolo[4,5-c]quinolin-4-amine

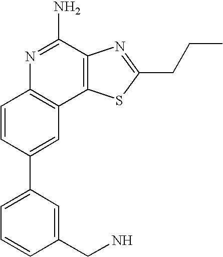

8-Bromo-2-propylthiazolo[4,5-c]quinolin-4-amine (0.500 g, 1.55 mmol) was coupled with 3-(aminomethyl)boronic acid hydrochloride (0.349 g, 1.86 mmol) using the method of Example 3 except that 2.2 equivalents of sodium carbonate was used. The crude product was purified by HPFC (silica gel, gradient elution with 0-35% CMA in chloroform) to provide an off-white solid. The solid was triturated with diethyl ether (10 mL), isolated by filtration, and then dried under vacuum at 100° C. to provide 0.270 g of 8-[3-(aminomethyl)phenyl]-2-propylthiazolo[4,5-c]quinolin-4-amine as an off-white solid, mp 189-191° C.; ¹H NMR (300 MHz, DMSO-d₆) δ 8.02 (d, J=1.8 Hz, 1 H), 7.85 (dd, J=8.7, 2.1 Hz, 1 H), 7.77 (s, 1 H), 7.68 (d, J=8.7 Hz, 1 H), 7.61 (d, J=7.8 Hz, 1 H), 7.41 (t, J=7.6 Hz, 1 H), 7.31 (d, J=7.6 Hz, 1 H), 6.94 (s, 2 H), 3.81 (s, 2 H), 3.18 (t, J=7.4 Hz, 2 H), 2.04 (bs, 2 H), 1.96-1.83 (m, 2 H), 1.04 (t, J=7.3 Hz, 3 H); ¹³C NMR (75 MHz, DMSO-d₆) δ 171.2, 152.6, 145.4, 144.7, 139.8, 138.2, 134.5, 129.0, 128.0, 126.8, 126.3, 125.8, 124.9, 122.4, 119.5, 46.1, 35.6, 23.1, 13.8; MS (ESI) m/z 349.15 (M+H)⁺; Anal. Calcd for C₂₀H₂₀N₄S: C, 68.94; H, 5.79; N, 16.08; Found: C, 68.68; H, 5.47; N, 15.98.

### Example 143

### 8-[4-(Aminomethyl)phenyl]-2-propylthiazolo[4,5-c]quinolin-4-amine

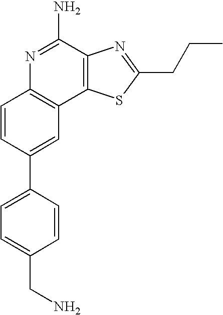

8-Bromo-2-propylthiazolo[4,5-c]quinolin-4-amine (0.500 g, 1.55 mmol) was coupled with 4-(aminomethyl)boronic acid hydrochloride (0.349 g, 1.86 mmol) using the method of Example 3 except that 2.2 equivalents of sodium carbonate was used. The crude product was purified by HPFC (silica gel, gradient elution with 0-35% CMA in chloroform) to provide an off-white solid. The solid was triturated with diethyl ether (10 mL), isolated by filtration, and then dried under vacuum at 100° C. to provide 0.141 g of 8-[4-(aminomethyl)phenyl]-2-propylthiazolo[4,5-c]quinolin-4-amine as an off-white solid, mp 225-226° C.; ¹H NMR (300 MHz, DMSO-d₆) δ 8.00 (d, J=2.0 Hz, 1 H), 7.83 (dd, J=8.7, 2.1 Hz, 1 H), 7.73 (d, J=8.2 Hz, 2 H), 7.67 (d, J=8.6 Hz, 1 H), 7.44 (d, J=8.2 Hz, 2 H), 6.92 (s, 2 H), 3.77 (s, 2 H), 3.18 (t, J=7.5 Hz, 2 H), 2.04 (bs, 2 H), 1.96-1.83 (m, 2 H), 1.04 (t, J=7.3 Hz, 3 H); ¹³C NMR (75 MHz, DMSO-d₆, 350° K) δ 171.2, 152.5, 144.6, 143.6, 139.7, 138.1, 134.6, 127.9, 127.8, 127.0, 126.8, 122.1, 119.8, 45.8, 35.7, 22.9, 13.7; MS (ESI) m/z 349.16 (M+H)⁺; C₂₀H₂₀N₄S: C, 68.94; H, 5.79; N, 16.08; Found: C, 68.74; H, 5.53; N, 16.09.

### Examples 144-146

The compounds in the table below were prepared and purified according to the methods of Examples 5-41. The table below shows the reagent used for each example, the structure of the resulting compound, and the observed accurate mass for the isolated trifluoroacetate salt.

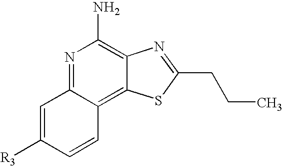

| Ex. | Reagent | R₃ | Measured Mass (M + H) |
|---|---|---|---|
| 144 | (2-Acetylaminophenyl)boronic acid | 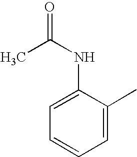 | 377.1438 |
| 145 | (3-Aminomethylphenyl)boronic acid, HCL | 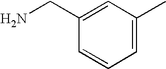 | 349.1517 |
| 146 | 3-(Methylsulfonylamino)phenylboronic acid | 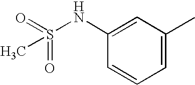 | 413.1084 |

### Examples 147-151

Part A

Triethylamine (161 g, 1.6 mole) was added to a suspension of 3-amino-7-bromoquinolin-4-ol hydrochloride (224.6 g, 0.8 mole) in dichloromethane (2.25 L). The mixture was cooled in an ice bath and acetoxy acetyl chloride was added dropwise over a period of 1 hour. The reaction mixture was stirred for 1 hour and then allowed to stand over the weekend. A solid was isolated by filtration. The solid was suspended in water (2 L), stirred for 2 hours, isolated by filtration, and then dried in an oven at 60-70° C. to provide 223 g of (7-bromo-4-hydroxyquinolin-3-ylcarbamoyl)methyl acetate as a reddish brown solid.

Part B

Phosphorous pentasulfide (146 g, 0.32 mol) was added to a suspension of the material from Part A in pyridine (2 L). The reaction mixture was heated at reflux for 1.5 hours. A portion (1.8 L) of the pyridine was removed by vacuum distillation. A 1:1 mixture of aqueous saturated sodium bicarbonate and water (1.5 L) was slowly added. Additional solvent (750 mL) was distilled off and then the reaction mixture was stirred overnight. A solid was isolated by filtration, washed with water (3×500 mL), and suction dried to provide 220 g of (7-bromothiazolo[4,5-c]quinolin-2-yl)methyl acetate as a brown solid.

Part C

3-Chloroperoxybenzoic acid (3.32 g of 77%, 14. 8 mmol) was added to a mixture of (7-bromothiazolo[4,5-c]quinolin-2-yl)methyl acetate (2 g, 6 mmol) and chloroform (60 mL). The reaction mixture was stirred for 2 hours and then washed with 10% aqueous sodium carbonate. The aqueous was extracted with dichloromethane (2×50 mL). The combined organics were dried over magnesium sulfate, filtered, and then concentrated under reduced pressure to provide crude (7-bromo-5-oxythiazolo[4,5-c]quinolin-2-yl)methyl acetate.

Part D

Trichloroacetyl isocyanate (1.8 mL, 15 mmol) was added dropwise to a mixture of the material from Part C and chloroform (60 mL). The reaction mixture was stirred at ambient temperature for 45 minutes. More trichloroacetyl isocyanate (1.8 mL) was added and the reaction mixture was stirred overnight. The reaction mixture was concentrated under reduced pressure. The residue was combined with ethanol (60 mL) and potassium ethoxide (499 mg) and stirred at ambient temperature over the weekend. The reaction mixture was heated at reflux overnight and then concentrated under reduced pressure. The residue was combined with ethanol (20 mL) and filtered. The isolated solid was washed with ethanol and diethyl ether and then dried to provide 1.68 g of (4-amino-7-bromothiazolo[4,5-c]quinolin-2-yl)methanol as a tan solid.

Part E

The compounds in the table below were prepared and purified using the methods described in Examples 5-41. (4-Amino-7-bromothiazolo[4,5-c]quinolin-2-yl)methanol was used in lieu of 7-bromo-2-propylthiazolo[4,5-c]quinolin-4-amine. The table below shows the reagent used for each example, the structure of the resulting compound, and the observed accurate mass for the isolated trifluoroacetate salt.

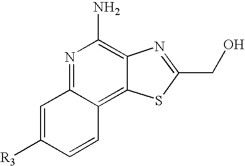

| Ex | Reagent | R₃ | Measured Mass (M + H) |
|---|---|---|---|
| 147 | 5-({[tert- Butyl(dimethyl)silyl]oxy}methyl)pyri- din-3-ylboronic acid | 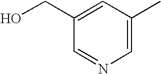 | 339.0926 |
| 148 | [3-(Hydroxypropyl)phenyl]boronic acid | 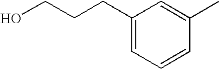 | 366.1313 |
| 149 | 3-(N,N- Dimethylaminocarbonyl)phenylboronic acid | 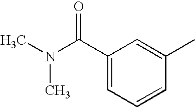 | 379.1237 |
| 150 | 3-(N- Isopropylaminocarbonyl)phenylboronic acid | 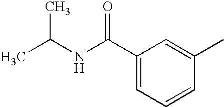 | 393.1368 |
| 151 | 3-(Pyrrolidine-1- carbonyl)phenylboronic acid | 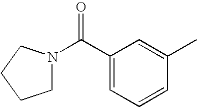 | 405.1383 |

### Examples 152-162

7-bromo-2-propylthiazolo[4,5-c][1,5]naphthyridin-4-amine was prepared according to the general methods of Example 1 Parts A through I using 5-bromopyridin-3-amine in lieu of 3-bromoaniline in Part A. The crude product was purified by HPFC (silica gel eluting with a gradient of 0-10% CMA in chloroform) to provide 0.66 g of a light yellow solid. This material was recrystallized from refluxing acetonitrile (25 mL), isolated by filtration, washed with cold acetonitrile, and dried in a vacuum oven at 60° C. to provide 475 mg of product as light yellow needles, mp 168-171° C. ¹H NMR (500 MHz, d₆-DMSO) δ 8.58 (d, J=2.2, 1H), 8.16 (d, J=1.9, 1H), 7.38 (s, 2H), 3.17 (d, J=7.2, 2H), 1.86 (sextet, J=7.6, 2H), 1.01 (t, J=7.5, 3H); ¹³C NMR (125 Hz, d₆-DMSO) δ 173.7, 153.0, 144.7, 141.3, 140.0, 139.4, 135.4, 1343, 118.9, 35.3, 22.8, 13.4; Anal. calcd for C₁₂H₁₁N₄SBr: C, 44.59; H, 3.48; N, 17.33. Found: C, 44.59; H, 3.15; N, 17.12.

The compounds in the table below were prepared and purified using the methods described in Examples 5-41. 7-Bromo-2-propylthiazolo[4,5-c][1,5]naphthyridin-4-amine was used in lieu of 7-bromo-2-propylthiazolo[4,5-c]quinolin-4-amine. The table below shows the reagent used for each example, the structure of the resulting compound, and the observed accurate mass for the isolated trifluoroacetate salt.

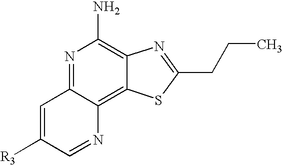

| Ex | Reagent | R₃ | Measured Mass (M + H) |
|---|---|---|---|
| 152 | (2-Hydroxymethylphenyl)boronic acid dihydrate | 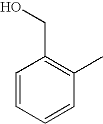 | 351.1306 |
| 153 | 4-(Hydroxymethyl)phenylboronic acid | 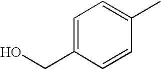 | 351.1273 |
| 154 | (3-Aminocarbonylphenyl)boronic acid | 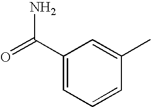 | 364.1224 |
| 155 | [3-(Hydroxypropyl)phenyl]boronic acid | 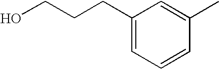 | 379.1570 |
| 156 | 3-(N,N- Dimethylaminocarbonyl)phenylboronic acid | 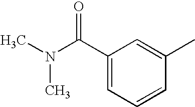 | 392.1551 |
| 157 | 3-(N- Isopropylaminocarbonyl)phenylboronic acid | 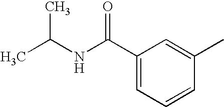 | 406.1711 |
| 158 | 3-(N- Propylaminocarbonyl)phenylboronic acid | 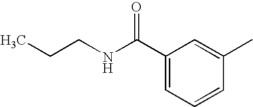 | 406.1707 |
| 159 | 3-(Methylsulfonylamino)phenylboronic acid | 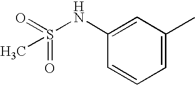 | 414.1084 |
| 160 | 3-(Morpholine-4- carbonyl)phenylboronic acid | 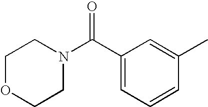 | 434.1662 |
| 161 | 4-(Morpholine-4- carbonyl)phenylboronic acid | 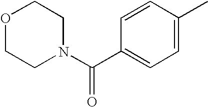 | 434.1677 |
| 162 | 5-({[tert- Butyl(dimethyl)silyl]oxy}methyl)pyri- din-3-ylboronic acid | 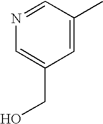 | 352.1245 |

Exemplary Compounds

Certain exemplary compounds, including some of those described above in the Examples, have the following Formulas (IIa and IIIb) and the following R₂ and R₃ substituents, wherein each line of the table below is matched with Formula IIa or IIIb to represent a specific compound.

| | |
|---|---|
| | IIa |
| 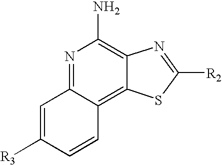 | |

| | |
|---|---|
| | IIIb |
| 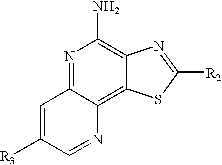 | |

| R₂ | R₃ |
|---|---|
| methyl | 3-aminocarbonylphenyl |
| methyl | 3-(N-propylaminocarbonyl)phenyl |
| methyl | 3-(N-isobutylaminocarbonyl)phenyl |
| methyl | 3-(N-isopropylaminocarbonyl)phenyl |
| methyl | 3-(N,N-dimethylaminocarbonyl)phenyl |
| methyl | 3-(piperidine-1-carbonyl)phenyl |
| methyl | 3-(morpholine-4-carbonyl)phenyl |
| methyl | 4-(morpholine-4-carbonyl)phenyl |
| methyl | 3-(N-cyclopentylaminocarbonyl)phenyl |
| methyl | 4-(methylsulfonyl)phenyl |
| methyl | 3-(methylsulfonylamino)phenyl |
| methyl | 3-(3-hydroxypropyl)phenyl |
| methyl | 2-(hydroxymethyl)phenyl |
| methyl | 5-hydroxymethylpyridin-3-yl |
| methyl | 4-(ethylsulfonyl)phenyl |
| methyl | 3-(hydroxymethyl)phenyl |
| methyl | 4-(hydroxymethyl)phenyl |
| methyl | 2-(acetylamino)phenyl |
| methyl | 4-(ethylaminocarbonylaminomethyl)phenyl |
| methyl | 4-(propylaminocarbonylaminomethyl)phenyl |
| methyl | 4-[(morpholine-4-carbonyl)aminomethyl]phenyl |
| methyl | 4-[(piperidine-1-carbonyl)aminomethyl]phenyl |
| methyl | 4-(isobutyrylaminomethyl)phenyl |
| ethyl | 3-aminocarbonyiphenyl |
| ethyl | 3-(N-propylaminocarbonyl)phenyl |
| ethyl | 3-(N-isobutylaminocarbonyl)phenyl |
| ethyl | 3-(N-isopropylaminocarbonyl)phenyl |
| ethyl | 3-(N,N-dimethylaminocarbonyl)phenyl |
| ethyl | 3-(piperidine-1-carbonyl)phenyl |
| ethyl | 3-(morpholine-4-carbonyl)phenyl |
| ethyl | 4-(morpholine-4-carbonyl)phenyl |
| ethyl | 3-(N-cyclopentylaminocarbonyl)phenyl |
| ethyl | 4-(methylsulfonyl)phenyl |
| ethyl | 3-(methylsulfonylamino)phenyl |
| ethyl | 3-(3-hydroxypropyl)phenyl |
| ethyl | 2-(hydroxymethyl)phenyl |
| ethyl | 5-hydroxymethylpyridin-3-yl |
| ethyl | 4-(ethylsulfonyl)phenyl |
| ethyl | 3-(hydroxymethyl)phenyl |
| ethyl | 4-(hydroxymethyl)phenyl |
| ethyl | 2-(acetylamino)phenyl |
| ethyl | 4-(ethylaminocarbonylaminomethyl)phenyl |
| ethyl | 4-(propylaminocarbonylaminomethyl)phenyl |
| ethyl | 4-{(morpholine-4-carbonyl)aminomethyl]phenyl |
| ethyl | 4-{(piperidine-1-carbonyl)aminomethyl]phenyl |
| ethyl | 4-(isobutyrylaminomethyl)phenyl |
| n-propyl | 3-aminocarbonylphenyl |
| n-propyl | 3-(N-propylaminocarbonyl)phenyl |
| n-propyl | 3-(N-isobutylaminocarbonyl)phenyl |
| n-propyl | 3-(N-isopropylaminocarbonyl)phenyl |
| n-propyl | 3-(N,N-dimethylaminocarbonyl)phenyl |
| n-propyl | 3-(piperidine-1-carbonyl)phenyl |
| n-propyl | 3-(morpholine-4-carbonyl)phenyl |
| n-propyl | 4-(morpholine-4-carbonyl)phenyl |
| n-propyl | 3-(N-cyclopentylaminocarbonyl)phenyl |
| n-propyl | 4-(methylsulfonyl)phenyl |
| n-propyl | 3-(methylsulfonylamino)phenyl |
| n-propyl | 3-(3-hydroxypropyl)phenyl |
| n-propyl | 2-(hydroxymethyl)phenyl |
| n-propyl | 5-hydroxymethylpyridin-3-yl |
| n-propyl | 4-(ethylsulfonyl)phenyl |
| n-propyl | 3-(hydroxymethyl)phenyl |
| n-propyl | 4-(hydroxymethyl)phenyl |
| n-propyl | 2-(acetylamino)phenyl |
| n-propyl | 4-(ethylaminocarbonylaminomethyl)phenyl |
| n-propyl | 4-(propylaminocarbonylaminomethyl)phenyl |
| n-propyl | 4-[(morpholine-4-carbonyl)aminomethyl]phenyl |
| n-propyl | 4-[(piperidine-1-carbonyl)aminomethyl]phenyl |
| n-propyl | 4-(isobutyrylaminomethyl)phenyl |
| methoxymethyl | 3-aminocarbonylphenyl |
| methoxymethyl | 3-(N-propylaminocarbonyl)phenyl |
| methoxymethyl | 3-(N-isobutylaminocarbonyl)phenyl |
| methoxymethyl | 3-(N-isopropylaminocarbonyl)phenyl |
| methoxymethyl | 3-(N,N-dimethylaminocarbonyl)phenyl |
| methoxymethyl | 3-(piperidine-1-carbonyl)phenyl |
| methoxymethyl | 3-(morpholine-4-carbonyl)phenyl |
| methoxymethyl | 4-(morpholine-4-carbonyl)phenyl |
| methoxymethyl | 3-(N-cyclopentylaminocarbonyl)phenyl |
| methoxymethyl | 4-(methylsulfonyl)phenyl |
| methoxymethyl | 3-(methylsulfonylamino)phenyl |
| methoxymethyl | 3-(3-hydroxypropyl)phenyl |
| methoxymethyl | 2-(hydroxymethyl)phenyl |
| methoxymethyl | 5-hydroxymethylpyridin-3-yl |
| methoxymethyl | 4-(ethylsulfonyl)phenyl |
| methoxymethyl | 3-(hydroxymethyl)phenyl |
| methoxymethyl | 4-(hydroxymethyl)phenyl |
| methoxymethyl | 2-(acetylamino)phenyl |
| methoxymethyl | 4-(ethylaminocarbonylaminomethyl)phenyl |
| methoxymethyl | 4-(propylaminocarbonylaminomethyl)phenyl |
| methoxymethyl | 4-[(morpholine-4-carbonyl)aminomethyl]phenyl |
| methoxymethyl | 4-[(piperidine-1-carbonyl)aminomethyl]phenyl |
| methoxymethyl | 4-(isobutyrylaminomethyl)phenyl |
| ethoxymethyl | 3-aminocarbonylphenyl |
| ethoxymethyl | 3-(N-propylaminocarbonyl)phenyl |
| ethoxymethyl | 3-(N-isobutylaminocarbonyl)phenyl |
| ethoxymethyl | 3-(N-isopropylaminocarbonyl)phenyl |
| ethoxymethyl | 3-(N,N-dimethylaminocarbonyl)phenyl |
| ethoxymethyl | 3-(piperidine-1-carbonyl)phenyl |
| ethoxymethyl | 3-(morpholine-4-carbonyl)phenyl |
| ethoxymethyl | 4-(morpholine-4-carbonyl)phenyl |
| ethoxymethyl | 3-(N-cyclopentylaminocarbonyl)phenyl |
| ethoxymethyl | 4-(methylsulfonyl)phenyl |
| ethoxymethyl | 3-(methylsulfonylamino)phenyl |
| ethoxymethyl | 3-(3-hydroxypropyl)phenyl |
| ethoxymethyl | 2-(hydroxymethyl)phenyl |
| ethoxymethyl | 5-hydroxymethylpyridin-3-yl |
| ethoxymethyl | 4-(ethylsulfonyl)phenyl |
| ethoxymethyl | 3-(hydroxymethyl)phenyl |
| ethoxymethyl | 4-(hydroxymethyl)phenyl |
| ethoxymethyl | 2-(acetylamino)phenyl |
| ethoxymethyl | 4-(ethylaminocarbonylaminomethyl)phenyl |
| ethoxymethyl | 4-(propylaminocarbonylaminomethyl)phenyl |
| ethoxymethyl | 4-[(morpholine-4-carbonyl)aminomethyl]phenyl |
| ethoxymethyl | 4-[(piperidine-1-carbonyl)aminomethyl]phenyl |
| ethoxymethyl | 4-(isobutyrylaminomethyl)phenyl |
| 2-methoxyethyl | 3-aminocarbonylphenyl |
| 2-methoxyethyl | 3-(N-propylaminocarbonyl)phenyl |
| 2-methoxyethyl | 3-(N-isobutylaminocarbonyl)phenyl |
| 2-methoxyethyl | 3-(N-isopropylaminocarbonyl)phenyl |
| 2-methoxyethyl | 3-(N,N-dimethylaminocarbonyl)phenyl |
| 2-methoxyethyl | 3-(piperidine-1-carbonyl)phenyl |
| 2-methoxyethyl | 3-(morpholine-4-carbonyl)phenyl |
| 2-methoxyethyl | 4-(morpholine-4-carbonyl)phenyl |
| 2-methoxyethyl | 3-(N-cyclopentylaminocarbonyl)phenyl |
| 2-methoxyethyl | 4-(methylsulfonyl)phenyl |
| 2-methoxyethyl | 3-(methylsulfonylamino)phenyl |
| 2-methoxyethyl | 3-(3-hydroxypropyl)phenyl |
| 2-methoxyethyl | 2-(hydroxymethyl)phenyl |
| 2-methoxyethyl | 5-hydroxymethylpyridin-3-yl |
| 2-methoxyethyl | 4-(ethylsulfonyl)phenyl |
| 2-methoxyethyl | 3-(hydroxymethyl)phenyl |
| 2-methoxyethyl | 4-(hydroxymethyl)phenyl |
| 2-methoxyethyl | 2-(acetylamino)phenyl |
| 2-methoxyethyl | 4-(ethylaminocarbonylaminomethyl)phenyl |
| 2-methoxyethyl | 4-(propylaminocarbonylaminomethyl)phenyl |
| 2-methoxyethyl | 4-[(morpholine-4-carbonyl)aminomethyl]phenyl |
| 2-methoxyethyl | 4-[(piperidine-1-carbonyl)aminomethyl]phenyl |
| 2-methoxyethyl | 4-(isobutyrylaminomethyl)phenyl |

### Cytokine Induction in Human Cells

Compounds of the invention have been found to modulate cytokine biosynthesis by inducing the production of interferon a and/or tumor necrosis factor a in human cells when tested using the method described below.

An in vitro human blood cell system is used to assess cytokine induction. Activity is based on the measurement of interferon (α) and tumor necrosis factor (α) (IFN-α and TNF-α, respectively) secreted into culture media as described by Testerman et. al. in “Cytokine Induction by the Immunomodulators Iniquimod and S-27609”, Journal of Leukocyte Biology, 58, 365-372 (September, 1995).

Blood Cell Preparation for Culture

Whole blood from healthy human donors is collected by venipuncture into vacutainer tubes or syringes containing EDTA. Peripheral blood mononuclear cells (PBMC) are separated from whole blood by density gradient centrifugation using HISTOPAQUE-1077 (Sigma, St. Louis, Mo.) or Ficoll-Paque Plus (Amersham Biosciences Piscataway, N.J.). Blood is diluted 1:1 with Dulbecco's Phosphate Buffered Saline (DPBS) or Hank's Balanced Salts Solution (HBSS). Alternately, whole blood is placed in Accuspin (Sigma) or LeucoSep (Greiner Bio-One, Inc., Longwood, Fla.) centrifuge frit tubes containing density gradient medium. The PBMC layer is collected and washed twice with DPBS or HBSS and re-suspended at 4×10⁶ cells/mL in RPMI complete. The PBMC suspension is added to 96 well flat bottom sterile tissue culture plates containing an equal volume of RPMI complete media containing test compound.

Compound Preparation

The compounds are solubilized in dimethyl sulfoxide (DMSO). The DMSO concentration should not exceed a final concentration of 1% for addition to the culture wells. The compounds are generally tested at concentrations ranging from 30-0.014 μM. Controls include cell samples with media only, cell samples with DMSO only (no compound), and cell samples with reference compound.

Incubation

The solution of test compound is added at 60 μM to the first well containing RPMI complete and serial 3 fold dilutions are made in the wells. The PBMC suspension is then added to the wells in an equal volume, bringing the test compound concentrations to the desired range (usually 30-0.014 μM). The final concentration of PBMC suspension is 2×10⁶ cells/mL. The plates are covered with sterile plastic lids, mixed gently and then incubated for 18 to 24 hours at 37° C. in a 5% carbon dioxide atmosphere.

Separation

Following incubation the plates are centrifuged for 10 minutes at 1000 rpm (approximately 200×g) at 4° C. The cell-free culture supernatant is removed and transferred to sterile polypropylene tubes. Samples are maintained at −30 to −70° C. until analysis. The samples are analyzed for IFN-α by ELISA and for TNF-α by IGEN/BioVeris Assay.

Interferon (α) and Tumor Necrosis Factor (α) Analysis

IFN-α concentration is determined with a human multi-subtype colorimetric sandwich ELISA (Catalog Number 41105) from PBL Biomedical Laboratories, Piscataway, N.J. Results are expressed in pg/mL.

The TNF-α concentration is determined by ORIGEN M-Series Immunoassay and read on an IGEN M-8 analyzer from BioVeris Corporation, formerly known as IGEN International, Gaithersburg, Md. The immunoassay uses a human TNF-α capture and detection antibody pair (Catalog Numbers AHC3419 and AHC3712) from Biosource International, Camarillo, Calif. Results are expressed in pg/mL.

Assay Data and Analysis

In total, the data output of the assay consists of concentration values of TNF-α and IFN-α (y-axis) as a function of compound concentration (x-axis).

Analysis of the data has two steps. First, the greater of the mean DMSO (DMSO control wells) or the experimental background (usually 20 pg/mL for IFN-α and 40 pg/mL for TNF-α) is subtracted from each reading. If any negative values result from background subtraction, the reading is reported as “*”, and is noted as not reliably detectable. In subsequent calculations and statistics, “*”, is treated as a zero. Second, all background subtracted values are multiplied by a single adjustment ratio to decrease experiment to experiment variability. The adjustment ratio is the area of the reference compound in the new experiment divided by the expected area of the reference compound based on the past 61 experiments (unadjusted readings). This results in the scaling of the reading (y-axis) for the new data without changing the shape of the dose-response curve. The reference compound used is 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91) and the expected area is the sum of the median dose values from the past 61 experiments.

The minimum effective concentration is calculated based on the background-subtracted, reference-adjusted results for a given experiment and compound. The minimum effective concentration (μmolar) is the lowest of the tested compound concentrations that induces a response over a fixed cytokine concentration for the tested cytokine (usually 20 pg/mL for IFN-α and 40 pg/mL for TNF-α). The maximal response is the maximal amount of cytokine (pg/ml) produced in the dose-response.

### Cytokine Induction in Human Cells (High Throughput Screen)

The CYTOKINE INDUCTION IN HUMAN CELLS test method described above was modified as follows for high throughput screening.

Blood Cell Preparation for Culture

Whole blood from healthy human donors is collected by venipuncture into vacutainer tubes or syringes containing EDTA. Peripheral blood mononuclear cells (PBMC) are separated from whole blood by density gradient centrifugation using HISTOPAQUE-1077 (Sigma, St. Louis, Mo.) or Ficoll-Paque Plus (Amersham Biosciences Piscataway, N.J.). Whole blood is placed in Accuspin (Sigma) or LeucoSep (Greiner Bio-One, Inc., Longwood, Fla.) centrifuge frit tubes containing density gradient medium. The PBMC layer is collected and washed twice with DPBS or HBSS and re-suspended at 4×10⁶ cells/mL in RPMI complete (2-fold the final cell density). The PBMC suspension is added to 96-well flat bottom sterile tissue culture plates.

Compound Preparation

The compounds are solubilized in dimethyl sulfoxide (DMSO). The compounds are generally tested at concentrations ranging from 30-0.014 μM. Controls include cell samples with media only, cell samples with DMSO only (no compound), and cell samples with a reference compound 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91) on each plate. The solution of test compound is added at 7.5 mM to the first well of a dosing plate and serial 3 fold dilutions are made for the 7 subsequent concentrations in DMSO. RPMI Complete media is then added to the test compound dilutions in order to reach a final compound concentration of 2-fold higher (60-0.028 μM) than the final tested concentration range.

Incubation

Compound solution is then added to the wells containing the PBMC suspension bringing the test compound concentrations to the desired range (usually 30-0.014 μM) and the DMSO concentration to 0.4%. The final concentration of PBMC suspension is 2×10⁶ cells/mL. The plates are covered with sterile plastic lids, mixed gently and then incubated for 18 to 24 hours at 37° C. in a 5% carbon dioxide atmosphere.

Separation

Following incubation the plates are centrifuged for 10 minutes at 1000 rpm (approximately 200 g) at 4° C. 4-plex Human Panel MSD MULTI-SPOT 96-well plates are pre-coated with the appropriate capture antibodies by MesoScale Discovery, Inc. (MSD, Gaithersburg, Md.). The cell-free culture supernatants are removed and transferred to the MSD plates. Fresh samples are typically tested, although they may be maintained at −30 to −70° C. until analysis.

Interferon-α and Tumor Necrosis Factor-α Analysis

MSD MULTI-SPOT plates contain within each well capture antibodies for human TNF-α and human IFN-α that have been pre-coated on specific spots. Each well contains four spots: one human TNF-α capture antibody (MSD) spot, one human IFN-α capture antibody (PBL Biomedical Laboratories, Piscataway, N.J.) spot, and two inactive bovine serum albumin spots. The human TNF-α capture and detection antibody pair is from MesoScale Discovery. The human IFN-α multi-subtype antibody (PBL Biomedical Laboratories) captures all IFN-α subtypes except IFN-α F (IFNA21). Standards consist of recombinant human TNF-α (R&D Systems, Minneapolis, Minn.) and IFN-α (PBL Biomedical Laboratories). Samples and separate standards are added at the time of analysis to each MSD plate. Two human IFN-α detection antibodies (Cat. Nos. 21112 & 21100, PBL) are used in a two to one ratio (weight:weight) to each other to determine the IFN-α concentrations. The cytokine-specific detection antibodies are labeled with the SULFO-TAG reagent (MSD). After adding the SULFO-TAG labeled detection antibodies to the wells, each well's electrochemoluminescent levels are read using MSD's SECTOR HTS READER. Results are expressed in pg/mL upon calculation with known cytokine standards.

Assay Data and Analysis

In total, the data output of the assay consists of concentration values of TNF-α or IFN-α (y-axis) as a function of compound concentration (x-axis).

A plate-wise scaling is performed within a given experiment aimed at reducing plate-to-plate variability associated within the same experiment. First, the greater of the median DMSO (DMSO control wells) or the experimental background (usually 20 pg/mL for IFN-α and 40 pg/mL for TNF-α) is subtracted from each reading. Negative values that may result from background subtraction are set to zero. Each plate within a given experiment has a reference compound that serves as a control. This control is used to calculate a median expected area under the curve across all plates in the assay. A plate-wise scaling factor is calculated for each plate as a ratio of the area of the reference compound on the particular plate to the median expected area for the entire experiment. The data from each plate are then multiplied by the plate-wise scaling factor for all plates. Only data from plates bearing a scaling factor of between 0.5 and 2.0 (for both cytokines IFN-α, TNF-α) are reported. Data from plates with scaling factors outside the above mentioned interval are retested until they bear scaling factors inside the above mentioned interval. The above method produces a scaling of the y-values without altering the shape of the curve. The reference compound used is 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91). The median expected area is the median area across all plates that are part of a given experiment.

A second scaling may also be performed to reduce inter-experiment variability (across multiple experiments). All background-subtracted values are multiplied by a single adjustment ratio to decrease experiment-to-experiment variability. The adjustment ratio is the area of the reference compound in the new experiment divided by the expected area of the reference compound based on an average of previous experiments (unadjusted readings). This results in the scaling of the reading (y-axis) for the new data without changing the shape of the dose-response curve. The reference compound used is 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91) and the expected area is the sum of the median dose values from an average of previous experiments.

The minimum effective concentration is calculated based on the background-subtracted, reference-adjusted results for a given experiment and compound. The minimum effective concentration (μmolar) is the lowest of the tested compound concentrations that induces a response over a fixed cytokine concentration for the tested cytokine (usually 20 pg/mL for IFN-α and 40 pg/mL for TNF-α). The maximal response is the maximal amount of cytokine (pg/ml) produced in the dose-response.

### TNF-α Inhibition in Mouse Cells

Certain compounds of the invention may modulate cytokine biosynthesis by inhibiting production of tumor necrosis factor α (TNF-α) when tested using the method described below.

The mouse macrophage cell line Raw 264.7 is used to assess the ability of compounds to inhibit tumor necrosis factor-α (TNF-α) production upon stimulation by lipopolysaccharide (LPS).

Single Concentration Assay:

Blood Cell Preparation for Culture

Raw cells (ATCC) are harvested by gentle scraping and then counted. The cell suspension is brought to 3×10⁵ cells/mL in RPMI with 10% fetal bovine serum (FBS). Cell suspension (100 μL) is added to 96-well flat bottom sterile tissues culture plates (Becton Dickinson Labware, Lincoln Park, N.J.). The final concentration of cells is 3×10⁴ cells/well. The plates are incubated for 3 hours. Prior to the addition of test compound the medium is replaced with colorless RPMI medium with 3% FBS.

Compound Preparation

The compounds are solubilized in dimethyl sulfoxide (DMSO). The DMSO concentration should not exceed a final concentration of 1% for addition to the culture wells. Compounds are tested at 5 μM. LPS (Lipopolysaccaride from Salmonella typhimurium, Sigma-Aldrich) is diluted with colorless RPMI to the EC₇₀ concentration as measured by a dose response assay.

Incubation

A solution of test compound (1 μl) is added to each well. The plates are mixed on a microtiter plate shaker for 1 minute and then placed in an incubator. Twenty minutes later the solution of LPS (1 μL, EC₇₀ concentration ˜10 ng/ml) is added and the plates are mixed for 1 minute on a shaker. The plates are incubated for 18 to 24 hours at 37° C. in a 5% carbon dioxide atmosphere.

TNF-α Analysis

Following the incubation the supernatant is removed with a pipet. TNF-α concentration is determined by ELISA using a mouse TNF-α kit (from Biosource International, Camarillo, Calif.). Results are expressed in pg/mL. TNF-α expression upon LPS stimulation alone is considered a 100% response.

Dose Response Assay:

Blood Cell Preparation for Culture

Raw cells (ATCC) are harvested by gentle scraping and then counted. The cell suspension is brought to 4×10⁵ cells/mL in RPMI with 10% FBS. Cell suspension (250 μL) is added to 48-well flat bottom sterile tissues culture plates (Costar, Cambridge, Mass.). The final concentration of cells is 1×10⁵ cells/well. The plates are incubated for 3 hours. Prior to the addition of test compound the medium is replaced with colorless RPMI medium with 3% FBS.

Compound Preparation

The compounds are solubilized in dimethyl sulfoxide (DMSO). The DMSO concentration should not exceed a final concentration of 1% for addition to the culture wells. Compounds are tested at 0.03, 0.1, 0.3, 1, 3, 5 and 10 μM. LPS (Lipopolysaccaride from Salmonella typhimurium, Sigma-Aldrich) is diluted with colorless RPMI to the EC₇₀ concentration as measured by dose response assay.

Incubation

A solution of test compound (200 μl) is added to each well. The plates are mixed on a microtiter plate shaker for 1 minute and then placed in an incubator. Twenty minutes later the solution of LPS (200 μL, EC₇₀ concentration ˜10 ng/ml) is added and the plates are mixed for 1 minute on a shaker. The plates are incubated for 18 to 24 hours at 37° C. in a 5% carbon dioxide atmosphere.

TNF-α Analysis

Following the incubation the supernatant is removed with a pipet. TNF-α concentration is determined by ELISA using a mouse TNF-α kit (from Biosource International, Camarillo, Calif.). Results are expressed in pg/mL. TNF-α expression upon LPS stimulation alone is considered a 100% response.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A compound of the Formula (I): 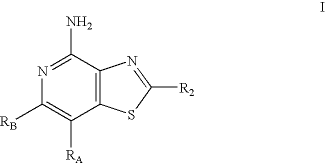
wherein:
R_{A} and R_{B} taken together form a fused benzene ring or fused pyridine ring wherein the benzene ring or pyridine ring is substituted by one R₃ group, or substituted by one R₃ group and one R group;
R₂ is selected from the group consisting of:
hydrogen,
alkyl,
hydroxyalkylenyl,
haloalkylenyl,
alkenyl,
alkyl-O-alkylenyl,
alkyl-O-alkenylenyl,
alkenyl-O-alkylenyl,
alkenyl-O-alkenylenyl,
N(R₈)₂-alkylenyl,
N₃-alkylenyl,
N(R₈)₂—C((O)—O-alkylenyl,
heterocyclyl,
heterocyclyl-O-alkylenyl,
heterocyclyl-O-alkenylenyl,
aryl,
aryl-O-alkylenyl,
aryl-O-alkenylenyl,
heteroaryl,
heteroaryl-O-alkylenyl, and
heteroaryl-O-alkenylenyl;
R₃ is selected from the group consisting of:
-Z-Ar,
-Z-Ar′—Y—R₄,
-Z-Ar′—X—Y—R₄,
-Z-Ar′—R₅, and
-Z-Ar′—X—R₅;
Ar is selected from the group consisting of aryl and heteroaryl both of which are substituted by one or more substituents independently selected from the group consisting of alkenyl, methylenedioxy, mercapto, carboxy, aryloxy, arylalkoxy, heteroaryloxy, heteroarylalkoxy, and alkyl wherein the alkyl group is substituted by one or more substituents selected from the group consisting of hydroxy, amino, alkylamino, dialkylamino, and carboxy;
Ar′ is selected from the group consisting of arylene and heteroarylene both of which can be unsubstituted or can be substituted by one or more substituents independently selected from the group consisting of alkyl, alkenyl, alkoxy, haloalkyl, haloalkoxy, halogen, nitro, hydroxy, hydroxyalkyl, mercapto, cyano, carboxy, formyl, aryl, aryloxy, arylalkoxy, heteroaryl, heteroaryloxy, heteroarylalkoxy, heterocyclyl, heterocyclylalkyl, amino, alkylamino, and dialkylamino;
X is selected from the group consisting of alkylene, alkenylene, alkynylene, arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O— groups;
Y is selected from the group consisting of:
—O—,
—S(O)₀₋₂—,
—S(O)₂—N(R₈)—,
—O—C(R₆)—,
—O—C(O)—O—,
—N(R₈)-Q-,
—C(R₆)—N(R₈)—,
—O—C(R₆)—N(R₈)—,
—C(R₆)—N(OR₉)—, 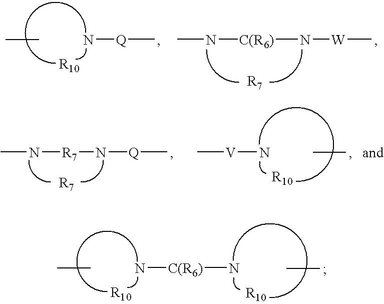
Z is selected from the group consisting of a bond, alkylene, alkenylene, and alkynylene;
R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;
R₅ is 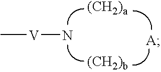
A is selected from the group consisting of —O—, —C(O)—, —S(O)₀₋₂—, —CH₂—, and —N(R₄)—;
Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;
V is selected from the group consisting of —C(R₆)—, —O—C(R₆)—, —N(R₈)—C(R₆)—, and —S(O)₂—;
W is selected from the group consisting of a bond, —C(O)—, and —S(O)₂—;
a and b are each an integer from 1 to 6 with the proviso that a+b is ≦7;
R₆ is selected from the group consisting of ═O and ═S;
R₇ is C₂₋₇ alkylene,
R₈ is selected from the group consisting of hydrogen, alkyl, alkoxyalkylenyl, and arylalkylenyl;
R₉ is selected from the group consisting of hydrogen and alkyl;
R₁₀ is independently C₃₋₈ alkylene; and
R is selected from the group consisting of fluoro, chloro, alkyl, alkoxy, hydroxy, and trifluoromethyl;
with the proviso that when R₃ is -Z-Ar′—Y—R and Y is —S—, then R₄ is other than alkyl; with the further proviso that when R₃ is -Z-Ar′—Y—R₄ and Y is —N(R₈)-Q- and R₈ is hydrogen or alkyl and Q is a bond, then R₄ is other than hydrogen or alkyl; with the further proviso that when R₃ is -Z-Ar′—Y—R₄ and Y is —O—, then R₄ is other than hydrogen, alkyl, or haloalkyl; and with the further proviso that when R₃ is -Z-Ar′—X—Y—R₄ and X is —CH₂— and Y is —O—, then R₄ is other than alkyl;
or a pharmaceutically acceptable salt thereof.

2. (canceled)

3. A compound of Formula (II): 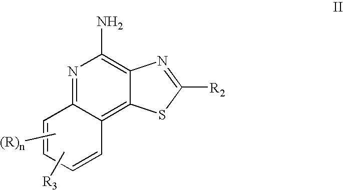
wherein:
R₂ is selected from the group consisting of:
hydrogen,
alkyl
hydroxyalkylenyl,
haloalkylenyl,
alkenyl,
alkyl-O-alkylenyl,
alkyl-O-alkenylenyl,
alkenyl-O-alkylenyl,
alkenyl-O-alkenylenyl,
N(R₈)₂-alkylenyl,
N₃-alkylenyl,
N(R₈)₂—C(O)—O-alkylenyl.
heterocyclyl,
heterocyclyl-O-alkylenyl,
heterocyclyl-O-alkenylenyl,
aryl,
aryl-O-alkylenyl,
aryl-O-alkenylenyl,
heteroaryl,
heteroaryl-O-alkylenyl, and
heteroaryl-O-alkenylenyl;
R₃ is selected from the group consisting of:
-Z-Ar,
-Z-Ar′—Y—R₄,
-Z-Ar′—X—Y—R₄,
-Z-Ar′—R₅, and
-Z-Ar′—X—R₅;
Ar is selected from the group consisting of aryl and heteroaryl both of which are substituted by one or :more substituents independently selected from the group consisting of alkenyl, methylenedioxy, mercapto, carboxy, aryloxy, arylalkoxy, heteroaryloxy, heteroarylalkoxy, and alkyl wherein the alkyl group is substituted by one or more substituents selected from the group consisting of hydroxy, amino, alkylamino, dialkylamino, and carboxy;
Ar′ is selected from the group consisting of arylene and heteroarylene both of which can be unsubstituted or can be substituted by one or more substituents independently selected from the group consisting of alkyl, alkenyl, alkoxy, haloalkyl, haloalkoxy, halogen, nitro, hydroxy, hydroxyalkyl, mercapto, cyano, carboxy, formyl, aryl, aryloxy, arylalkoxy, heteroaryl, heteroaryloxy, heteroarylalkoxy, heterocyclyl, heterocyclylalkyl, amino, alkylamino, and dialkylamino;
X is selected from the group consisting of alkylene, alkenylene, alkynylene, arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O— groups;
Y is selected from the group consisting of:
—O—,
—S(O)₀₋₂—,
—S(O)₂—N(R₈)—,
—O—C(R₆)—,
—O—C(O)—O—,
—N(R₈)-Q-,
—C(R₆)—N(R₈)—,
—O—C(R₆)—N(R₈)—,
—C(R₆)—N(OR₉)—, 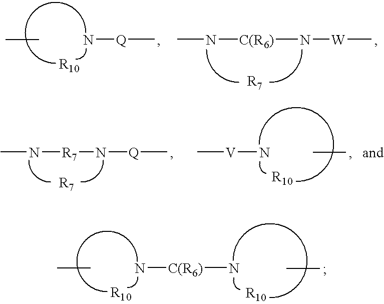
Z is selected from the group consisting of a bond, alkylene, alkenylene, and alkynylene;
R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;
R₅ is 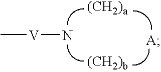
A is selected from the group consisting of —O—, —C(O)—, —S(O)₀₋₂—, —CH₂—, and —N(R₄)—;
Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;
V is selected from the group consisting of —C(R₆)—, —O—C(R₆)—, —N(R₈)—C(R₆)—, and —S(O)₂—;
W is selected from the group consisting of a bond, —C(O)—, and —S(O)₂—;
a and b are each an integer from 1 to 6 with the proviso that a+b is ≦7;
R₆ is selected from the group consisting of ═O and ═S;
R₇ is C₂₋₇ alkylene;
R₈ is selected from the group consisting of hydrogen, alkyl, alkoxyalkylenyl, and arylalkylenyl;
R₉ is selected from the group consisting of hydrogen and alkyl;
R₁₀ is independently C₃₋₈ alkylene;
R is selected from the group consisting of fluoro, chloro, alkyl, alkoxy, hydroxy, and trifluoromethyl; and
n is 0 or 1;
with the proviso that when R₃ is -Z-Ar′—Y—R₄ and Y is —S—, then R₄ is other than alkyl; with the further proviso that when R₃ is -Z-Ar′—Y—R₄ and Y is —N(R₈)-Q- and R₈ is hydrogen or alkyl and Q is a bond, then R₄ is other than hydrogen or alkyl; with the further proviso that when R₃ is -Z-Ar′—Y—R₄ and Y is —O—, then R₄ is other than hydrogen, alkyl, or haloalkyl; and with the further proviso that when R₃ is -Z-Ar′—X—Y—R₄ and X is —CH₂— and Y is —O—, then R₄ is other than alkyl;
or a pharmaceutically acceptable salt thereof.

4. (canceled)

5. The compound or salt of claim 1 wherein the compound is of the following Formula (III): 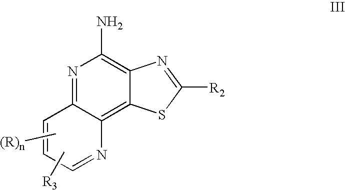
wherein:
R₂ is selected from the group consisting of:
hydrogen,
alkyl,
hydroxyalkylenyl,
haloalkylenyl,
alkenyl,
alkyl-O-alkylenyl,
alkyl-O-alkenylenyl,
alkenyl-O-alkylenyl,
alkenyl-O-alkenylenyl,
N(R₈)₂-alkylenyl,
N₃-alkylenyl,
N(R₈)₂—C(O)—O-alkylenyl,
heterocyclyl,
heterocyclyl-O-alkylenyl,
heterocyclyl-O-alkenylenyl,
aryl,
aryl-O-alkylenyl,
aryl-O-alkenylenyl,
heteroaryl,
heteroaryl-O-alkylenyl, and
heteroaryl-O-alkenylenyl;
R₃ is selected from the group consisting of:
-Z-Ar,
-Z-Ar′—Y—R₄,
-Z-Ar′—X—Y—R₄,
-Z-Ar′—R₅, and
-Z-Ar′—X—R₅;
Ar is selected from the group consisting of aryl and heteroaryl both of which are substituted by one or more substituents independently selected from the group consisting of alkenyl, methylenedioxy, mercapto, carboxy, aryloxy, arylalkoxy, heteroaryloxy, heteroarylalkoxy, and alkyl wherein the alkyl group is substituted by one or more substituents selected from the group consisting of hydroxy, amino, alkylamino, dialkylamino, and carboxy;
Ar′ is selected from the group consisting of arylene and heteroarylene both of which can be unsubstituted or can be substituted by one or more substituents independently selected from the group consisting of alkyl, alkenyl, alkoxy, haloalkyl, haloalkoxy, halogen, nitro, hydroxy, hydroxyalkyl, mercapto, cyano, carboxy, formyl, aryl, aryloxy, arylalkoxy, heteroaryl, heteroaryloxy, heteroarylalkoxy, heterocyclyl, heterocyclylalkyl, amino, alkylamino, and dialkylamino;
X is selected from the group consisting of alkylene, alkenylene, alkynylene, arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O— groups;
Y is selected from the group consisting of:
—O—,
—S(O)₀₋₂—,
—S(O)₂—N(R₈)—,
—O—C(R₆)—,
—O—C(O)—O—,
—N(R₈)-Q-,
—C(R₆)—N(R₈)—,
—O—C(R₆)—N(R₈)—,
—C(R₆)—N(OR₉)—, 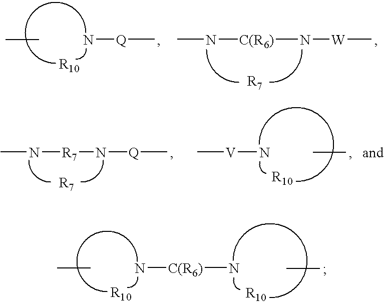
Z is selected from the group consisting of a bond, alkylene, alkenylene and alkynylene;
R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;
R₅ is 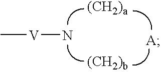
A is selected from the group consisting of —O—, —C(O)—, —S(O)₀₋₂—, —CH₂—, and —N(R₄)—;
Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;
V is selected from the group consisting of —C(R₆)—, —O—C(R₆)—, —N(R₈)—C(R₆)—, and —S(O)₂—;
W is selected from the group consisting of a bond, —C(O)—, and —S(O)₂—;
a and b are each an integer from 1 to 6 with the proviso that a+b is ≦7;
R₆ is selected from the group consisting of ═O and ═S;
R₇ is C₂₋₇ alkylene;
R₈ is selected from the group consisting of hydrogen, alkyl, alkoxyalkylenyl, and arylalkylenyl;
R₉ is selected from the group consisting of hydrogen and alkyl;
R₁₀ is independently C₃₋₈ alkylene;
R is selected from the group consisting of fluoro, chloro, alkyl, alkoxy, hydroxy, and trifluoromethyl; and
n is 0 or 1;
with the proviso that when R₃ is -Z-Ar′—Y—R₄ and Y is —S—, then R₄ is other than alkyl; with the further proviso that when R₃ is -Z-Ar′—Y—R₄ and Y is —N(R₈)-Q- and R₈ is hydrogen or alkyl and Q is a bond, then R₄ is other than hydrogen or alkyl; with the further proviso that when R₃ is -Z-Ar′—Y—R₄ and Y is —O—, then R₄ is other than hydrogen, alkyl, or haloalkyl; and with the further proviso that when R₃ is -Z-Ar′—X—Y—R₄ and X is —CH₂— and Y is —O—, then R₄ is other than alkyl;
or a pharmaceutically acceptable salt thereof.

6. (canceled)

7. The compound or salt of claim 3 wherein R₃ is attached at the 7-position.

8. The compound or salt of claim 3 wherein n is 0.

9. The compound or salt of claim 1 wherein R₃ is selected from the group consisting of phenyl, pyridyl, pyrrolyl, thienyl, and furyl; each of which is substituted by one or more substituents selected from the group consisting of alkenyl, hydroxyalkylenyl, aminoalkylenyl, methylenedioxy, carboxy, and arylalkyleneoxy.

10-12. (canceled)

13. The compound or salt of claim 1 wherein R₃ is -Z-Ar, and Ar is phenyl or pyridyl, wherein the phenyl or pyridyl group is substituted by one HO—C₁₋₄ alkylenyl.

14-16. (canceled)

17. The compound or salt of claim 1 wherein R₃ is -Z-Ar′—Y—R₄.

18. (canceled)

19. The compound or salt of claim 17 wherein:
Y is selected from the group consisting of —S(O)₂—, —NH-Q-, and —C(O)—N(R₈)—,
wherein:
Q is selected from the group consisting of —C(O)—, —C(O)—NH—, and —S(O)₂—; and
R₈ is hydrogen or C₁₋₄ alkyl; and
R₄ is hydrogen or C₁₋₆ alkyl.

20. The compound or salt of claim 17 wherein Ar′ is phenylene.

21. (canceled)

22. The compound or salt claim 1 wherein R₃ is -Z-Ar′—X—Y—R₄.

23. (canceled)

24. The compound or salt of claim 22 wherein:
Y is selected from the group consisting of —S(O)₂—, —NH-Q-, and —C(O)—N(R₈)—,
wherein:
Q is selected from the group consisting of —C(O)—, —C(O)—NH—, and —S(O)₂—;
R₈ is hydrogen or C₁₋₄ alkyl; and
R₄ is hydrogen or C₁₋₆ alkyl.

25. The compound or salt of claim 22 wherein Ar′ is phenylene.

26-28. (canceled)

29. The compound or salt of claim 1 wherein Z is a bond,

30. (canceled)

31. The compound or salt of claim 1 wherein R₂ is hydrogen, C₁₋₄ alkyl or C₁₋₄ alkyl-O—C₁₋₄ alkylenyl.

32. (canceled)

33. A compound of the Formula (VII): 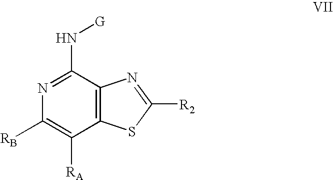
wherein:
R_{A} and R_{B} taken together form a fused benzene ring or fused pyridine ring wherein the benzene ring or pyridine ring is substituted by one R₃ group, or substituted by one R₃ group and one R group;
R₂ is selected from the group consisting of:
hydrogen,
alkyl,
hydroxyalkylenyl,
haloalkylenyl,
alkenyl,
alkyl-O-alkylenyl,
alkyl-O-alkenylenyl,
alkenyl-O-alkylenyl,
alkenyl-O-alkenylenyl,
N(R₈)₂-alkylenyl,
N₃-alkylenyl,
N(R₈)₂—C(O)—O-alkylenyl,
heterocyclyl,
heterocyclyl-O-alkylenyl,
heterocyclyl-O-alkenylenyl,
aryl,
aryl-O-alkylenyl,
aryl-O-alkenylenyl,
heteroaryl,
heteroaryl-O-alkylenyl, and
heteroaryl-O-alkenylenyl;
R₃ is selected from the group consisting of:
-Z-Ar,
-Z-Ar′—Y—R₄,
-Z-Ar′—X—Y—R₄,
-Z-Ar′—R₅, and
-Z-Ar′—X—R₅—;
Ar is selected from the group consisting of aryl and heteroaryl both of which are substituted by one or more substituents independently selected from the group consisting of alkenyl, methylenedioxy, mercapto, carboxy, aryloxy, arylalkoxy, heteroaryloxy, heteroarylalkoxy, and alkyl wherein the alkyl group is substituted by one or more substituents selected from the group consisting of hydroxy, amino, alkylamino, dialkylamino, and carboxy;
Ar′ is selected from the group consisting of arylene and heteroarylene both of which can be unsubstituted or can be substituted by one or more substituents independently selected from the group consisting of alkyl, alkenyl, alkoxy, haloalkyl, haloalkoxy, halogen, nitro, hydroxy, hydroxyalkyl, mercapto, cyano, carboxy, formyl, aryl, aryloxy, arylalkoxy, heteroaryl, heteroaryloxy, heteroarylalkoxy, heterocyclyl, heterocyclylalkyl, amino, alkyl amino, and dialkylamino;
X is selected from the group consisting of alkylene, alkenylene, alkynylene,
arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O— groups;
Y is selected from the group consisting of:
—O—,
—S(O)₀₋₂—,
—S(O)₂—N(R₈)—,
—O—C(R₆)—,
—O—C(O)—O—,
—N(R₈)-Q-,
—C(R₆)—N(R₈)—,
—O—C(R₆)—N(R₈)—,
—C(R₆)—N(OR₉)—, 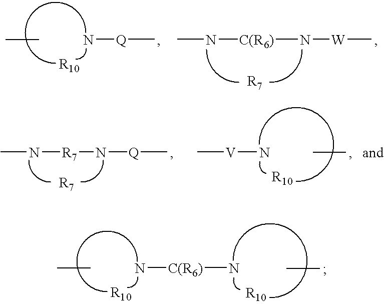
Z is selected from the group consisting of a bond, alkylene, alkenylene, and alkynylene;
R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo; 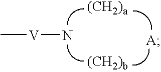
A is selected from the group consisting of —O—, —C(O)—, —S(O)₀₋₂—, —CH₂—, and —N(R₄)—;
Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, and —C(R₆)—N(OR₉)—;
V is selected from the group consisting of —C(R₆)—, —O—C(R₆)—, —N(R₈)—C(R₆)—, and —S(O)₂—;
W is selected from the group consisting of a bond, —C(O)—, and —S(O)₂—;
a and b are each an integer from 1 to 6 with the proviso that a+b is ≦7;
R₆ is selected from the group consisting of ═O and ═S;
R₇ is C₂₋₇ alkylene;
R₈ is selected from the group consisting of hydrogen, alkyl, alkoxyalkylenyl, and arylalkylenyl;
R₉ is selected from the group consisting of hydrogen and alkyl;
R₁₀ is independently C₃₋₈ alkylene; and
R is selected from the group consisting of fluoro, chloro, alkyl, alkoxy, hydroxy, and trifluoromethyl;
G is selected from the group consisting of:
—C(O)—R′,
α-aminoacyl,
α-aminoacyl-α-aminoacyl,
—C(O)—O—R′,
—C(O)—N(R″)R′,
—CH(OH)—C(O)—OY′,
—CH(OC₁₋₄alkyl)Y₀,
—CH₂Y₁, and
—CH(CH₃)Y₁,
R′ and R″ are independently selected from the group consisting of C₁₋₁₀ alkyl, C₃₋₇ cycloalkyl, and benzyl, each of which may be unsubstituted or substituted by one or more substituents selected from the group consisting of halogen, hydroxy, nitro, cyano, carboxy C₁₋₆ alkyl, C₁₋₄ alkoxy, aryl, heteroaryl, arylC₁₋₄ alkylenyl, heteroarylC₁₋₄ alkylenyl, haloC₁₋₄ alkylenyl, haloC₁₋₄ alkoxy, —O—C(O)—CH₃, —C(O)—O—CH₃, —C(O)—NH₂, —O—CH₂—C(O)—NH₂, —NH₂, and —S(O)₂—NH₂, with the proviso that R″ can also be hydrogen;
α-aminoacyl is an acyl group derived from an amino acid selected from the group consisting of racemic, D-, and L-amino acids;
Y′ is selected from the group consisting of hydrogen, C₁₋₆ alkyl, and benzyl;
Y₀ is selected from the group consisting of C₁₋₆ alkyl, carboxyC₁₋₆ alkylenyl, aminoC₁₋₄ alkylenyl, mono-N—C₁₋₆ alkylaminoC₁₋₄ alkylenyl, and di-N,N—C₁₋₆ alkylaminoC₁₋₄ alkylenyl;
Y₁ is selected from the group consisting of mono-N—C₁₋₆ alkylamino, di-N,N—C₁₋₆ alkylamino, morpholin-4-yl, piperidin-1-yl, pyrrolidin-1-yl, and 4-C₁₋₄ alkylpiperazin-1-yl;
with the proviso that when R₃ is -Z-Ar′—Y—R₄ and Y is —S—, then R₄ is other than alkyl; with the further proviso that when R₃ is -Z-Ar′—Y—R₄ and Y is —N(R₈)-Q- and R₈ is hydrogen or alkyl and Q is a bond, then R₄ is other than hydrogen or alkyl; with the further proviso that when R₃ is -Z-Ar′—Y—R₄ and Y is —O—, then R₄ is other than hydrogen, alkyl, or haloalkyl; and with the further proviso that when R₃ is -Z-Ar′—X—Y—R₄ and X is —CH₂— and Y is —O—, then R₄ is other than alkyl;
or a pharmaceutically acceptable salt thereof.

34. A pharmaceutical composition comprising a therapeutically effective amount of a compound or salt claim 1 and a pharmaceutically acceptable carrier.

35. A method of inducing cytokine biosynthesis in an animal comprising administering an effective amount of a compound or salt of claim 1 to the animal.

36. A method of treating a viral disease in an animal in need thereof comprising administering an effective amount of a compound or salt of claim 1 to the animal.

37. A method of treating a neoplastic disease in an animal in need thereof comprising administering an effective amount of a compound or salt of claim 1 to the animal.
